SEQUENCE LISTING (14 sequences)

SEQ ID NO: 1 - DNA, 2601 bp, Zika virus
atcaggtgcataggagtcagcaatagggactttgtggaaggtatgtcaggtgggacttgggttgatgtcgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgttgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaggaaggagcagttcacacggcccttgctggagctctggaggctgagatggatggtgcaaagggaaggctgtcctctggccacttgaaatgtcgcctgaaaatggacaaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgtggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttctgggctaaagatggctgttggtatggaatggagataaggcccaggaaagaaccagaaagcaacttagtaaggtcaatggtgactgcaggatcaactgatcacatggatcacttctccctt

SEQ ID NO: 2 - DNA, 10807 bp, Zika virus
agttgttgatctgtgtgaatcagactgcgacagttcgagtttgaagcgaaagctagcaacagtatcaacaggttttattttggatttggaaacgagagtttctggtcatgaaaaacccaaagaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaataataaagaagtttaagaaagatctggctgccatgctgagaataatcaatgctaggaaggagaagaagagacgaggcacagatactagtgtcggaattgttggcctcctgctgaccacagccatggcagtggaggtcactagacgtgggaatgcatactatatgtacttggacagaagcgatgctggggaggccatatcttttccaaccacaatggggatgaataagtgttatatacagatcatggatcttggacacatgtgtgatgccaccatgagctatgaatgccctatgctggatgagggggtagaaccagatgacgtcgattgttggtgcaacacgacgtcaacttgggttgtgtacggaacctgccaccacaaaaaaggtgaagcacggagatctagaagagctgtgacgctcccctcccattccactaggaagctgcaaacgcggtcgcagacctggttggaatcaagagaatacacaaagcacctgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgttaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcttgctctaagaaaatgaccgggaagagcatccagccagagaatctggagtaccggataatgctgtcagttcatggctcccagcacagtgggatgatcgttaatgatacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcatgctggggcagacaccggaactccacactggaacaacaaagaagcactggtagagttcaaggacgcacatgccaaaaggcagactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcactaagatcccggctgaaacactgcacgggacagtcacagtggaggtacagtacgcagggacagatggaccttgcaaggttccagctcagatggcggtggacatgcaaactctgaccccagttgggaggttgataaccgctaaccctgtaatcactgaaagcactgagaactccaagatgatgctggaactggatccaccatttggggactcttacattgtcataggagtcggggaaaagaagatcacccaccactggcacaggagtggcagcaccattggaaaagcatttgaagccactgtgagaggtgccaagagaatggcagtcttgggagacacagcctgggactttggatcagttgggggtgctctcaactcactgggcaagggcatccatcaaatttttggagcagctttcaaatcattgtttggaggaatgtcctggttctcacaaattctcattggaacgttgctggtgtggttgggtctgaatacaaagaatggatctatttcccttatgtgcttggccttagggggagtgttgatcttcttatccacagccgtctctgctgatgtggggtgctcggtggacttctcaaagaaggaaacgagatgcggtacaggggtgttcgtctataacgacgttgaagcttggagggacaggtacaagtaccatcctgactcccctcgtagattggcagcagcagtcaagcaagcctgggaagatgggatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccatggctggaaggcttgggggaaatcgtacttcgtcagggcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgtcattggaacagccgctaagggaaaggaggctgtgcacagtgatctaggctactggattgagagtgagaagaacgacacatggaggctgaagagggcccacctgatcgagatgaaaacatgtgaatggccaaagtcccacacattgtggacagatggaatagaagaaagtgatctgatcatacccaagtctttagctgggccactcagccatcacaacaccagagagggctacaggacccaaatgaaagggccatggcatagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggttgttggtatggaatggagataaggcccaggaaagaaccagaaagtaacttagtaaggtcaatggtgactgcaggatcaactgatcacatggatcacttctcccttggagtgcttgtgattctgctcatggtacaggaagggctaaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgttgctcatctggcgctgatagcggcattcaaagtcagacctgcgttgctggtatctttcattttcagagctaattggacaccccgtgagagcatgctgctggccttggcctcgtgtcttctgcaaactgcgatctccgccttggaaggcgacctgatggttcccatcaatggttttgctttggcctggttggcaatacgagcgatggttgttccacgcactgacaacatcaccttggcaatcctggctgctctgacaccactggcccggggcacactgcttgtggcgtggagagcaggccttgctacttgcggggggttcatgctcctttctctgaaggggaaaggcagtgtgaagaagaacttaccatttgtcatggccctgggactaaccgctgtgaggctggtcgaccccatcaacgtggtgggactgctgttgctcacaaggagtgggaagcggagctggccccctagtgaagtactcacagctgttggcctgatatgcgcattggctggagggttcgccaaggcggatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcactagatgagagtggtgatttctccctagtggaggatgatggtccccccatgagagagatcatactcaaagtggtcctgatggccatctgtggcatgaacccaatagccataccctttgcagctggagcgtggtacgtgtatgtgaagactggaaaaaggagtggtgctctatgggatgtgcctgctcccaaggaagtaaaaaagggggagaccacagatggagtgtacagagtaatgactcgtagactgctaggttcaacacaagttggagtgggagtcatgcaagagggggtcttccacactatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcagttgcgctggactacccagcaggaacttcaggatctccaatcctagataagtgtgggagagtgataggactctatggtaatggggtcgtgatcaaaaatgggagttacgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgcactgtgatcttagctccaaccagggttgtcgctgctgaaatggaggaagcccttagagggcttccagtgcgttatatgacaacagcagtcaatgtcacccattctgggacagaaatcgttgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgactgccacgccaccaggaacccgtgacgcattcccggactccaactcaccaattatggacaccgaagtggaagtcccagagagagcctggagctcaggctttgattgggtgacggatcattctggaaaaacagtttggtttgttccaagcgtgaggaatggcaatgagatcgcagcttgtctgacaaaggctggaaaacgggtcatacagctcagcagaaagacttttgagacagagttccagaaaacaaaacatcaagagtgggacttcgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaaccccaacaaacctggagatgagtatctgtatggaggtgggtgcgcagagactgatgaagaccatgcacactggcttgaagcaagaatgcttcttgacaacatttacctccaagatggcctcatagcctcgctctatcgacctgaggccgacaaagtagcagctattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagagtttgccgctgggaaaagaggagcggcctttggagtgatggaagccctgggaacactgccaggacatatgacagagagattccaggaggccattgacaacctcgctgtgctcatgcgggcagagactggaagcaggccctacaaagccgcggcggcccaattaccggagaccctagagactatcatgcttttggggttgctgggaacagtctcgctgggaatctttttcgtcttgatgcggaacaagggcatagggaagatgggctttggaatggtgactcttggggccagcgcatggcttatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtgggtcttctgggcttgattaccgccaatgaactcggatggttggagagaacaaagagtgacctaagccatctaatgggaaggagagaggagggggcaactataggattctcaatggacattgacctgcggccagcctcagcttgggctatctatgctgctctgacaactttcattaccccagccgtccaacatgcagtgaccacttcatacaacaactactccttaatggcgatggccacgcaagctggagtgttgttcggtatgggtaaagggatgccattctatgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagctgtctccagcgccatactgtcgcggaccgcctgggggtggggtgaggctggggccctgatcacagctgcaacttccactttgtgggagggctctccgaacaagtactggaactcctccacagccacctcactgtgtaacatttttaggggaagctacttggctggagcttctctaatctacacagtaacaagaaacgctggcttggtcaagagacgtgggggtggaacgggagagaccctgggagagaaatggaaggcccgcctgaaccagatgtcggccctggagttctactcctacaaaaagtcaggcatcaccgaggtgtgcagagaagaggcccgccgcgccctcaaggacggtgtggcaacgggaggccacgctgtgtcccgaggaagtgcaaagctgagatggttggtggagaggggatacctgcagccctatggaaaggtcattgatcttggatgtggcagagggggctggagttactatgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgatataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctccttttggggcgcatggacgggcccaggaggccagtgaaatatgaagaggatgtgaatctcggctctggcacgcgggctgtggtaagctgcgctgaagctcccaacatgaagatcattggtaaccgcattgagaggatccgcagtgagcacgcggaaacgtggttctttgacgagaaccacccatataggacatgggcttaccatggaagctacgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagttaggcaaacacaaacggccacgagtctgtaccaaagaagagttcatcaacaaggttcgtagcaacgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtacatgtggctaggggctagatttctagagttcgaagcccttggattcttgaacgaggatcactggatggggagagagaattcaggaggtggtgttgaagggctaggattacaaagactcggatatgtcttagaagagatgagtcgcataccaggaggaaggatgtatgcagatgatactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcaagacaagaccaaagggggagcggacaagttgtcacttacgctcttaatacatttaccaacctagtggtgcagctcattcggaatatggaggctgaggaagttctagagatgcaagacttgtggctgctgcggaggtcagagaaagtgaccaactggttgcagagcaatggatgggataggctcaaacgaatggcagtcagtggagatgattgcgttgtgaaaccaattgatgataggtttgcacatgctctcaggttcttgaatgatatgggaaaagttaggaaggacacacaagagtggaagccctcaactggatgggacaactgggaagaagttccgttttgctcccaccacttcaacaagctccatctcaaggacgggaggtccattgtggttccctgccgccaccaagatgaactgattggccgagctcgcgtctcaccgggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtgggtctctcatagggcacagaccgcgcaccacctgggctgagaacattaaaaacacagtcaacatgatgcgtaggatcataggtgatgaagaaaagtacgtggactacctatccacccaagttcgctacttgggcgaagaagggtccacacctggagtgctataagcaccaatcttagtgttgtcaggcctgctagtcagccacagcttggggaaagctgtgcagcctgtgacccccccaggagaagctgggaaaccaagcccatagtcaggccgagaacgccatggcacggaagaagccatgctgcctgtgagcccctcagaggacactgagtcaaaaaaccccacgcgcttggaggcgcaggatgggaaaagaaggtggcgaccttccccaccctttaatctggggcctgaactggagatcagctgtggatctccagaagagggactagtggttagaggagaccccccggaaaacgcaaaacagcatattgacgctgggaaagaccagagactccatgagtttccaccacgctggccgccaggcacagatcgccgaatagcggcggccggtgtggggaaatccatgggtct

SEQ ID NO: 3 - DNA, 10272 bp, Zika virus
atgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagccttcttgagattcacggcaatcaagccatcactgggtctcatcaatagatggggttcagtggggaaaaaagaggctatggaaataataaagaagttcaagaaagatctggctgccatgctgagaataatcaatgctaggaaggagaagaagagacgaggcgcagatactaatgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccactaggaagctgcaaacgcggtcgcaaacttggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcggacatggcttcggacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaggttgagataacgcccaattcaccaagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtaccgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggctgataaccgctaaccccgtaatcactgaaagcactgagaactccaagatgatgctggaacttgatccaccatttggggactcttacattgtcataggagtcggggagaagaagatcacccaccactggcacaggagtggcagcaccattggaaaagcatttgaagccactgtgagaggtgccaggagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgtgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtacttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcaccaaggtccacgtggaggaaacatgtggaacaagaggaccatctctgagatcaaccacagcaagcggaagggtgatcgaggaatggtgctgcagggagtgcacaatgcccccactgtcgttccaggctaaagatggctgttggtatggaatggagataaggcccaggaaagaaccagaaagtaacttagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggcttgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggagctctatgggatgtgcctgctcccaaggaagtaaaaaagggggagaccacagatggagtgtacagagtgatgactcgtagactgctaggttcaacacaagttggagtgggagttatgcaagagggggtctttcacaccatgtggcacgtcacaaaaggatccgcgctgagaagcggtgaagggagacttgatccatactggggagatgtcaagcaggatctggtgtcatactgtggtccatggaagctagatgccgcctgggacgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaatgggagttatgttagtgccatcacccaagggaggagggaggaagagactcctgttgagtgcttcgagccttcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgccgaaatggaggaagcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtctggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcacgcggccctgaagtcattcaaggagtttgccgctgggaaaagaggagcggcttttggagtgatggaagccttgggaacactgccaggacacatgacagagagattccaggaagccattgacaacctcgctgtgctcatgcgggcagagactggaagcaggccttacaaagccgcggcggcccaattgccggagaccctagagaccattatgcttttggggttgctgggaacagtctcgctgggaatctttttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctacgctgccttgacaactttcattaccccagccgtccaacatgcagtgaccacttcatacaacaactactccttaatggcgatggccacgcaagctggagtgttgtttggtatgggcaaagggatgccattctacgcatgggactttggagtcccgctgctaatgataggttgctactcacaattaacacccctgaccctaatagtagccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggccctgatcacagctgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccatgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctttccatggtgggggattggcttgaaaaaagaccaggagccttttgtataaaagtgttgtgtccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcccaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagatccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaagacagttatggacattatttcgagacaagaccaaagggggagcggacaagttgtcacttacgctcttaacacatttaccaacctagtggtgcaactcattcggagtatggaggctgaggaagttctagagatgcaagacttgtggctgctgcggaggtcagagaaagtgaccaactggctgcagagcaacggatgggataggctcaaacgaatggcagtcagtggagatgattgcgttgtgaggccaattgatgataggtttgcacatgccctcaggttcttgaatgatatggggaaagttaggaaggacacacaagagtggaaaccctcaactggatgggacaactgggaggaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaa

SEQ ID NO: 4 - DNA, 392 bp, Artificial Sequence
  A synthetic oligonucleotide
atcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaac

SEQ ID NO: 5 - DNA, 4251 bp, Artificial Sequence
  A synthetic oligonucleotide
  misc_feature (1)...(4251): n = A,T,C or G
tgttgatactcatactcttcctttttcatattattgaagcatttatcagggttattgtctcatgagccnnnnnnnnnnnnnnnnnnnnnnnnnnnnnnnnanaantnngggttccccgcncattnccccgaaaagtgccacctgacgtcgncggatcgggagatctccnnnnnnnnnnnnnnnnnnnnnnnnnnnnnnnngctctgatgccgcatagttaagccagtatctgctccctgcttgtgtgttggaggtcgctgagtagtgcnnnnnnnnnnnnnnnnnnnnnnnnnnnnnnnncttgaccgacaattgcatgaagaatctgcttagggttaggcgttttgcgctgcttcgcgatgtacggcnnnnnnnnnnnnnnnnnnnnnnnnnnnnnnnnagttattaatagtaatcaattacggggtcattagttcatagcccatatatggagttccgcgttacatannnnnnnnnnnnnnnnnnnnnnnnnnnnnnnncgaacgacccccgcccattgacgtcaataatgacgtatgttcccatagtaacgccaatagggactttcnnnnnnnnnnnnnnnnnnnnnnnnnnnnnnnnaactgcccacttggcagtacatcaagtgtatcatatgccaagtccgccccctattgacgtcaatgaccnnnnnnnnnnnnnnnnnnnnnnnnnnnnnnnnaactgcccacttggcagtacatcaagtgtatcatatgccaagtccgccccctattgacgtcaatgaccnnnnnnnnnnnnnnnnnnnnnnnnnnnnnnnnagcggtttgactcacggggatttccaagtctccaccccattgacgtcaatgggagtttgttttggcacnnnnnnnnnnnnnnnnnnnnnnnnnnnnnnnntaaccccgccccgttgacgcaaatgggcggtaggcgtgtacggtgggaggtctatataagcagagctcnnnnnnnnnnnnnnnnnnnnnnnnnnnnnnnncatccacgctgttttgacctccatagaagacaccgggaccgatccagcctccgcggccgggaacggtcnnnnnnnnnnnnnnnnnnnnnnnnnnnnnnnnactcaccgtccggatctcagcaagcaggtatgtactctccagggtgggcctggcttccccagtcaagannnnnnnnnnnnnnnnnnnnnnnnnnnnnnnnctcttacatgtaccttttgcttgcctcaaccctgactatcttccaggtcaggatcccagagtcagggcnnnnnnnnnnnnnnnnnnnnnnnnnnnnnnnngaacagtaaaccctgctccgaatattgcctctcacatctcgtcaatctccgcgaggactggggaccctnnnnnnnnnnnnnnnnnnnnnnnnnnnnnnnncctcctgctgaccacagctatggcagcggaggtcactagacgtgggagtgcatactatatgtacttgcnnnnnnnnnnnnnnnnnnnnnnnnnnnnnnnnccaaccacattggggatgaataagtgttatatacagatcatggatcttggacacatgtgtgatgccacnnnnnnnnnnnnnnnnnnnnnnnnnnnnnnnngggtggaaccagatgacgtcgattgttggtgcaacacgacgtcaacttgggttgtgtacggaacctgcnnnnnnnnnnnnnnnnnnnnnnnnnnnnnnnnaagagctgtgacgctcccctcccattccactagtttgctgcaaacgcggtcgcaaacctggttggaatnnnnnnnnnnnnnnnnnnnnnnnnnnnnnnnngaaaattggatattcaggaaccctggcttcgcgttagcagcagctgccatcgcttggcttttgggaatnnnnnnnnnnnnnnnnnnnnnnnnnnnnnnnntgatactgctgattgccccggcatacagcatcaggtgcataggagtcagcaatagggactttgtggaannnnnnnnnnnnnnnnnnnnnnnnnnnnnnnncttggaacatggaggttgtgtcaccgtaatggcacaggacaaaccgactgtcgacatagagctggttannnnnnnnnnnnnnnnnnnnnnnnnnnnnnnntcctactgctatgaggcatcaatatcggacatggcttcggacagccgctgcccaacacaaggtgaagcnnnnnnnnnnnnnnnnnnnnnnnnnnnnnnnntctgcaaaagaacgttagtggacagaggctggggaaatggatgtggactttttggcaaagggagcctcnnnnnnnnnnnnnnnnnnnnnnnnnnnnnnnnaatgaccgggaagagcatccagccagagaatctggagtaccggataatgctgtcagttcatggctcccnnnnnnnnnnnnnnnnnnnnnnnnnnnnnnnncatgaaactgatgagaatagagcgaaggttgagataacgcccaattcaccaagagccgaagccaccctnnnnnnnnnnnnnnnnnnnnnnnnnnnnnnnnaaccgaggacaggccttgacttttcagatttgtattacttgactatgaataacaagcactggttggttnnnnnnnnnnnnnnnnnnnnnnnnnnnnnnnnttggcacgctggggcagacaccggaactccacactggaacaacaaagaagcactggtagagttcaagcnnnnnnnnnnnnnnnnnnnnnnnnnnnnnnnnctagggagtcaagaaggagcagttcacacggcccttgctggagctctggaggctgagatggatggtgcnnnnnnnnnnnnnnnnnnnnnnnnnnnnnnnngtcgcctgaaaatggataaacttagattgaagggcgtgtcatactccttgtgtaccgcagcgttcacannnnnnnnnnnnnnnnnnnnnnnnnnnnnnnngacagtcacagtggaggtacagtacgcagggacagatggaccttgcaaggttccagctcagatggcgcnnnnnnnnnnnnnnnnnnnnnnnnnnnnnnnnttgataaccgctaaccccgtaatcactgaaagcactgagaactctaagatgatgctggaacttgatccnnnnnnnnnnnnnnnnnnnnnnnnnnnnnnnntcggggagaagaagatcacccaccactggcacaggagtggcagcaccattggaaaagcatttgaagccnnnnnnnnnnnnnnnnnnnnnnnnnnnnnnnngggagacacagcctgggactttggatcagttggaggcgctctcaactcattgggcaagggcatccatcnnnnnnnnnnnnnnnnnnnnnnnnnnnnnnnnggaggaatgtcctggttctcacaaattctcattggaacgttgctgatgtggttgggtctgaacacaaannnnnnnnnnnnnnnnnnnnnnnnnnnnnnnntagggggagtgttgatcttcttatccacagctgtctctgctgatgtggggtgctcggtgtgaggatctnnnnnnnnnnnnnnnnnnnnnnnnnnnnnnnnccctaaacttcatgggttacgtaattggaagttgggggacattgccacaagatcatattgtacaaaacnnnnnnnnnnnnnnnnnnnnnnnnnnnnnnnncaggcctattgattggaaagtatgtcaaaggattgtgggtcttttgggctttgctgctccatttacacnnnnnnnnnnnnnnnnnnnnnnnnnnnnnnnngcatgtatacaagctaaacaggctttcactttctcgccaacttacaaggcctttctaagtaaacagtannnnnnnnnnnnnnnnnnnnnnnnnnnnnnnnctggtctgtgccaagtgtttgctgacgcaacccccactggctggggcttggccataggccatcagcgcnnnnnnnnnnnnnnnnnnnnnnnnnnnnnnnnccatactgcggaactcctagccgcttgttttgctcgcagccggtctggagcaaagctcataggaactcnnnnnnnnnnnnnnnnnnnnnnnnnnnnnnnntcgtttcgatctacgtatgatctttttccctctgccaaaaattatggggacatcatgaagccccttgannnnnnnnnnnnnnnnnnnnnnnnnnnnnnnnttttcattgcaatagtgtgttggaattttttgtgtctctcactcggaaggaattctgcattaatgaatnnnnnnnnnnnnnnnnnnnnnnnnnnnnnnnnttgggcgctcttccgcttcctcgctcactganctcgntgcgcttcggtcgt

SEQ ID NO: 6 - protein, 867 aa, Zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFWAKDGCWYGMEIRPRKEPESNLVRSMVTAGSTDHMDHFSL

SEQ ID NO: 7 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGTDTSVGIVGLLLTTAMAVEVTRRGNAYYMYLDRSDAGEAISFPTTMGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLVWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAAKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVPINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMAICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRYGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMMRRIIGDEEKYVDYLSTQVRYLGEEGSTPGVL

SEQ ID NO: 8 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTNVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGARRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFQAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRSMEAEEVLEMQDLWLLRRSEKVTNWLQSNGWDRLKRMAVSGDDCVVRPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 9 - protein, 3423 aa, Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHMCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNTKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPMLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 10 - protein, 6 aa, Zika virus
KEKKRR

SEQ ID NO: 11 - DNA, 10251 bp, Zika virus
atgaaaaacccaaagaagaaatccggaggattccggattgtcaatatgctaaaacgcggagtagcccgtgtaaaccccttggggggtttgaagaggctgccggccggactcctgctgggccatggacccatcagaatggttttggcgatactagccttcttgagattcacagcaatcaagccatcactgggcctcatcaatagatggggttccgtggggaagaaggaggctatggaaataataaaaaagttcaagaaagatcttgctgccatgttgagaataatcaatgctaggaaggagaggaagagacgtggagctgatgccagcatcggaatcgtcagcctcctgctgactacagtcatggcagcagagatcactagacgcgggagtgcatactacatgtacttggacaggagcgatgctggtaaggccatttctttcgttaccacactgggggtgaacaaatgccatgtgcagatcatggacctcgggcatatgtgtgacgccaccatgagttatgagtgccccatgctggacgagggagtggagccagatgacgtcgattgctggtgcaacacgacatcaacttgggttgtgtacggaacctgtcatcataaaaaaggtgaagcacgacgatccagaagagccgtgacgcttccttctcactctacaaggaagttgcaaacgcgatcgcagacttggctagaatcaagagaatacacaaagcacctgatcaaggttgagaattggatattcaggaaccccgggtttgcgctagtggctgtagctattgcctggctcctgggaagctcgacgagccaaaaagtcatatacttggtcatgatattgttgattgccccggcatacagtatcaggtgcataggagttagcaatagagacttcgtggagggcatgtcaggtgggacctgggttgatgttgtcttggaacatggaggttgtgtcaccgtgatggcacaggacaagccaacagttgacatagagttggtcacgacaacggttagcaacatggccgaggtgagatcctactgctacgaggcatcaatatcggacatggcttcggacagtcgctgcccaacacaaggtgaagcctaccttgacaagcagtcagacactcaatatgtctgtaaaagaacattggtggacagaggttggggaaatgggtgtggactttttggcaaggggagcttggtgacgtgtgccaagtttacatgctccaagaaaatgacagggaagagcatccagccggagaacttggagtaccggataatgctatcagtgcatggatcccagcacagtgggatgattgtgaatgacgaaaacagagcaaaagtcgaggttacacccaattcaccaagagcagaagcaaccttgggaggttttggaagcctgggacttgattgtgaaccaaggacaggccttgacttttcagatctgtattacctgaccatgaacaataagcattggttggtgcacaaagagtggtttcatgacatcccattaccttggcattctggtgcagacactgaaactccacactggaacaacaaagaggcactggtggagttcaaggacgcccacgccaagaggcaaactgttgtggttctggggagccaagaaggagccgttcacacggctctcgctggagctctggaggctgagatggatggtgcgaagggaaggctatcctcaggccatttgaaatgccgcctaaaaatggacaagcttaggttgaagggtgtgtcatattccctgtgtaccgcagcgttcacattcaccaaggttccagctgaaacattgcatggaacagtcacagtggaggtgcagtatgcagggagggatggaccctgcaaggtcccagcccagatggcggtggacatgcagaccctgaccccagttggaaggctgataacggctaaccctgtgatcactgaaagcactgagaattcaaagatgatgttggagctcgacccaccatttggggattcttacattgtcataggagtcggggacaagaaaatcacccatcactggcatcggagtggtagcatcatcggaaaggcatttgaagccactgtgagaggcgccaagagaatggcagtcttgggagacacagcctgggactttggatcagttgggggtgtgtttaactcattgggcaagggtattcaccagatctttggagcagctttcaaatcactgttcggaggaatgtcctggttctcacagatcctcataggcacactgttggtgtggttgggtctgaacacaaagaatggatctatctccctcacatgcttggccttgggaggagtgatgatcttcctttccacggctgtttctgctgatgtggggtgttcggtggacttctcaaaaaaggaaacgagatgtggcacgggggtgttcatctacaatgacgttgaagcctggagggatcgatacagataccatcctgactccccccgcagattggcagcagctgttaagcaggcttgggaagaggggatttgtgggatctcctccgtttcgagaatggaaaacatcatgtggaaatcagtggaaggggagcttaatgcgatcctagaggagaatggagtccaactgacagttgtagtggggtctgtaaaaaaccccatgtggagaggtccacgaagattgccagtgcccgtaaatgagctgccccatggctggaaagcctgggggaaatcgtactttgttagggcggcaaagaccaacaacagttttgttgtcgacggtgacacactgaaggaatgtccgctcaaacatagagcatggaatagcttccttgtggaggatcacgggtttggggtcttccacaccagtgtttggctgaaggtcagagaggactattcattagagtgtgacccagccgtcataggaacagctgtcaagggaaaggaggctgcacacagtgatctaggctattggattgagagtgaaaagaatgacacatggaggctgaagagggctcatctgattgagatgaagacatgtgagtggccaaagtctcacacactgtggacagatggagtggaagaaagtgatctgatcatacccaagtccttagctggtccactcagccaccacaacaccagagagggttatagaactcaagtgaaagggccatggcatagtgaagagctcgaaatccggtttgaggaatgcccaggcaccaaggttcatgtggaggagacatgcggaactagaggaccatctttaagatcaaccactgcaagtggaagggtcatagaggaatggtgctgtagggaatgcacaatgcctccactatcgttccgggcaaaagacggctgctggtatggaatggagataaggcccagaaaggaaccagagagcaacttagtgaggtctatggtgacagcaggatcaaccgatcacatggatcacttctctcttggagtgcttgtgattctactcatggtgcaggaaggtttgaagaagagaatgaccacaaagatcataatgagcacatcaatggcaatgctggtagccatggtcttgggaggattctcaatgagtgacctggctaagcttgtgatcctgatgggtgccactttcgcagaaatgaacactggaggagatgtggctcacttggcattggtagcggcatttaaagtcagaccagccttgttggtttccttcatcttcagagccaactggacaccccgtgagagcatgctgctagccctggcttcgtgtctcctgcagactgcgatttccgctcttgaaggcgagctgatggtcctcgttaatggatttgctttggcctggttggcaatacgagcaatggccgtgccacgcactgataacatcgctctagcaattctggccgctctaacaccattagccagaggcacactgcttgtggcatggagagcgggcctcgccacttgtggagggttcatgctcatttccctgaaagggaaaggtagtgtgaagaagaacctgccatttgtcatggccttggggttgaccgctgtgaggatagtggaccccattaatgtggtaggactactgttactgacaaggagtgggaaacggagctggccccctagtgaagtgcttacagctgtcggcctgatatgtgcactggccggagggtttgccaaggcagacatagagatggctgggcccatggctgcagtaggcctgctaattgtcagttatgtggtcacgggaaagagtgtggacatgtacattgaaagagcaggtgatattacatgggaaaaagacgcggaagtcactggaaacagtcctcggcttgacgtggcactagatgagagtggtgatttctctttggtagaggaggatggcccacccatgagagagatcatactcaaggtggtcctgatggccatctgtggcatgaacccaatagccatacccttcgctgcaggagcgtggtatgtgtatgtaaagactgggaaaaggagcggtgccctctgggacgtgcctgctcccaaagaagtaaaaaagggagagactacagatggagtgtacagagttatgactcgcagactgctgggttcaacacaggttggagtgggagtcatgcaagagggagtcttccataccatgtggcacgtcacaaaaggagccgcattgaggagcggtgaaggaagacttgatccatactggggggacgtcaagcaggacctggtgtcatattgtgggccgtggaagttggatgcagcctgggatggactaagtgaggtgcagcttttggccgtaccccccggagagagggctaaaaacattcagactctgcctggaatatttaagacaaaggatggggacatcggagcagttgctctagactaccctgcaggaacctcaggatctccgatcctagacaaatgcggaagagtgataggactttatggcaatggggttgtgatcaagaatggaagctatgttagtgccataacccagggaaaaagggaggaggagactccggttgagtgctttgaaccctcgatgctgaggaagaagcagctaacagtcttggatctgcatccaggagccgggaaaaccaggagggttcttcctgaaatagtccgtgaagccataaagaagagacttcgcacagtgatcttagcaccaaccagggttgttgctgctgagatggaggaagccctaagaggacttccggtgcgttacatgacaacagcagtcaacgtcacccattctgggacagaaatcgttgatttgatgtgccatgccaccttcacttcacgcctactacaaccaatcagagtccccaactacaacctttatatcatggatgaggctcatttcacagatccttcaagcatagctgcaagaggatacatatcaacaagggttgaaatgggcgaggcggctgctatcttcatgactgctacaccaccaggaacccgcgatgcgtttccagattccaactcaccaatcatggacacagaagtggaagtcccagagagagcctggagctcaggctttgactgggtgacggaccattctggaaaaacaatttggtttgttccaagtgtgagaaacggaaatgaaatcgcagcctgtctgacaaaggctggaaagcgggttatacagctcagcaggaagacttttgagacagagtttcagaagacaaaaaatcaagagtgggactttgtcataacaactgacatttcagagatgggtgccaacttcaaggctgaccgggtcatagattccaggagatgcctaaagccagtcatacttgatggtgagagagtcatcctggctgggcctatgcccgtcacgcacgccagtgctgctcagaggagaggacgtataggcaggaaccccaacaaacctggagatgagtatatgtatggaggtgggtgtgcagagactgatgaagaccatgcacactggcttgaagcaagaatgcttctcgacaacatttacctccaggatggcctcatagcctcgctctatcggcctgaggctgacaaggttgccgccattgagggagagttcaagctgaggacagagcaaaggaagacctttgtggaactcatgaagagaggagaccttcccgtttggctggcctatcaagtagcatctgccggaataacttacacagacagaagatggtgctttgatggcactaccaacaacaccataatggaagacagtgtaccagcagaggtgtggaccaagtatggagagaagagagtgctcaaaccgaggtggatggatgccagggtctgttcagatcatgcggctttgaagtcgttcaaagaatttgccgctgggaagagaggagcggctttgggagtaatggatgccctaggaacattgccaggacacatgacagagaggtttcaggaagccattgacaatctcgctgtgctcatgcgagcagagactggaagtaggccctacaaagcagcggcagctcaactgccggagaccctagagaccattatgctcttgggtttattgggaacagtttcgctagggatcttctttgtcttgatgcggaacaagggcatcgggaagatgggcttcggaatggtaacccttggggccagcgcatggctcatgtggctttcggaaattgaaccagccagaatcgcatgtgtcctcattgtcgtgtttctgttactggtggtgctcatacctgagccagagaagcaaagatctccccaggacaatcaaatggcaatcatcatcatggtggcagtgggccttctgggtttgataactgcaaacgaactcggatggctggaaagaacaaaaagtgatatagctcatctaatgggaaggaaagaagaggggacaaccgtaggattctcaatggatattgatctgcggccagcctccgcctgggctatttatgccgcattgacaactctcatcaccccagccgtccaacatgcggtgaccacctcatacaacaactactccctgatggcgatggccacacaagctggagtgctgtttggcatgggcaaagggatgccattttatgcatgggactttggagtcccgctgctaatgatgggttgttactcacaattaacacccctgaccctgatagtggccatcattctgcttgtggcacactacatgtatttgatcccaggtttgcaggcagcagcagcacgtgccgcccagaagaggacagcagctggcatcatgaagaatcccgttgttgatggaatagtggtgactgacattgacacaatgacaattgacccccaagtggagaagaagatgggacaagtgttactcatagcagtagctgcctccagtgccgtgctgctgcggaccgcttggggatggggggaggctggggctctgatcacagcagcaacctccaccttatgggaaggctctccaaacaaatactggaactcctctacagccacttcactgtgcaatatcttcagaggaagttatttggcaggggcttcccttatttacacagtgacaagaaatgccggtctggttaagagacgtggaggtggaacgggagagactctgggagagaagtggaaagcccgcctgaaccagatgtcggctttggagttctattcttacaaaaagtcaggcatcaccgaagtgtgtagggaggaggcacgccgcgccctcaaggatggagtggccacaggaggacatgctgtatcccggggaagcgcaaagcttagatggttggtagagagaggatacctgcagccccatggaaaggttgttgacctcggatgtggcagagggggctggagttattacgctgccaccatccgtaaagtgcaggaggtcagaggatacacaaagggaggtcctggtcatgaagaacccatgctggtgcaaagctatgggtggaacatagttcgcctcaagagtggagtggacgtctttcacatggcggctgagccgtgtgacactttgctgtgtgacattggcgagtcatcgtccagtcctgaagtggaagagacgcgaacactcagagtgctctccatggtgggagactggctcgagaaaagaccaggggccttctgcataaaggtgctgtgcccatacaccagtactatgatggagaccatggagcgactgcaacgtaggtatgggggaggattggtcagagtgccattgtcccgcaactccacacatgagatgtattgggtctctggagccaaaagtaacatcataaagagtgtgtccaccacaagtcagctcctcttgggacgcatggatgggcctaggaggccagtgaaatatgaagaggatgtgaacctcggctcaggcacacgagctgtggcaagctgtgctgaggctcccaacatgaagatcattggtaggcgcattgagagaatccgcaatgaacatgcagagacatggttctttgatgaaaaccacccatacaggacatgggcctaccatgggagctacgaagcccccacgcaggggtcagcgtcatccctcgtgaacggggttgttagactcctgtcaaagccctgggatgtggtgactggagtcacaggaatagctatgactgacaccacgccatacggccaacaaagagtcttcaaagaaaaggtggacactagggtgccagacccccaagaaggcacccgccgagtaatgaacatggtctcgtcttggctatggaaggagctgggaaaacgcaagcggccacgtgtctgcaccaaagaagagttcatcaataaggtgcgcagcaatgcagcactgggagcaatatttgaagaggaaaaagaatggaagacagctgtagaagctgtgaatgatccgagattttgggctctagtggacaaggaaagagaacaccacctgagaggagagtgtcacagctgtgtgtacaacatgatgggaaaaagagaaaagaagcaaggagaattcgggaaagcaaaaggcagccgcgcaatctggtacatgtggttgggagccagatttctggagtttgaggctcttggattcttgaatgaggaccattggatgggaagagaaaactcaggaggtggcgttgaagggctaggactgcaaaggcttggatacattctagaagaaatgaaccgggcgccaggaggaaagatgtatgcagatgacaccgctggctgggatacccgtattagcaggtttgatctggagaatgaagccctgatcactaaccagatggaagaagggcacagagctctggcgttggccgtgattaaatacacataccaaaacaaagtggtgaaggttctcagaccagctgaaggagggaaaacagtcatggacatcatctcaagacaagaccagagagggagcggacaagttgttacttatgctctcaacacattcaccaacctggtggtgcagcttatccggaacatggaggctgaggaggtgctagagatgcatgatctatggctgttgaggaagccagagaaagtgaccagatggttgcagagcaatggatgggacagactcaaacgaatggcagtcagtggagatgactgcgttgtaaagccaattgatgataggtttgcacatgccctcaggttcttgaatgacatgggaaaagttaggaaagacacacaggaatggaaaccctcgactggatggagcaattgggaagaagtcccgttctgttcccaccacttcaacaagctgcacctcaaggatgggagatccattgtggtcccctgccgccaccaagatgaactgattggccgagcccgtgtctcaccaggggcaggatggagcatccgggagactgcctgtcttgcaaaatcatatgcccagatgtggcagcttctttatttccacagaagagacctccgactgatggccaatgccatctgttcggccgtgccagccgactgggtcccaactgggagaaccacctggtcaatccatggaaagggagaatggatgactaatgaggacatgctcatggtgtggaatagagtgtggattgaggagaacgaccacatgggggacaagacccctgtaacaaaatggacagacattccctatttgggaaaaagggaggacttatggtgtggatcccttatagggcacagacctcgcaccacttgggctgagaacatcaaagacacagtcaacatggtgcgtaggatcataggtgatgaagaaaggtacatggactacctatccacccaggtacgctacttgggtgaggaggggtccacacctggagtgctg

SEQ ID NO: 12 - DNA, 10269 bp, Zika virus
  misc_feature (1)...(10269): n = A,T,C or G
atgaaaaacccaaaaaagaaatccggaggattccggattgtcaatatgctaaaacgcggagtagcccgtgtgagcccctttgggggcttgaagaggctaccagctggacttctgctgggtcatggacccatcaggatggtcttggcgatactagccttcttgagattcacggcaatcaagccatcactgggtctcatcaatagatggggttccgtggggaaaaaagaggctatggaaataataaagaagttcaagaaagatctggctgccatgctgagaataatcaatgctaggaaggagaagaagagacgtggcgcagacaccagtgtcggaattgttggcctcctgctgaccacagccatggcagtggaggtcaccagacgtgggagtgcatactatatgtacttagacagaagcgatgctggggaggccatatcttttccaaccacactgggggtgaataagtgttacatacagatcatggatcttggacacatgtgtgatgccacaatgagctatgaatgccctatgttggatgagggggtagaaccagatgacgtcgattgctggtgcaacacgacatcgacttgggttgtgtacggaacctgccatcacaaaaaaggtgaggcacggagatctagaagagctgtgacgctcccctctcattccactaggaagctgcaaacgcggtcgcagacctggttggaatcaagagaatacacaaagcacttgatcagagtcgaaaattggatattcaggaaccctggctttgcgttggcagcagctgccattgcttggcttttgggaagctcaacgagccaaaaagtcatatacttggtcatgatactgttgattgccccggcatacagtatcaggtgcataggagtcagcaatagggattttgtggaaggtatgtcaggtgggacctgggttgatgttgtcttggaacatggaggttgtgttaccgtaatggcacaggacaagccaactgttgatatagagttggtcacaacaacggttagcaacatggcggaggtaagatcctactgctacgaggcatcaatatcggacatggcttcggacagccgctgcccaacacaaggtgaagcctaccttgacaagcagtcagacactcaatatgtttgcaaaagaacgttagtggacagaggttggggaaatggatgtggactctttggcaaagggagcctggtgacatgcgccaagtttgcatgctccaagaaaatgactgggaagagcatccagccagagaacctggagtaccggataatgctgtcagttcatggctcccagcacagtgggatgattgttaatgacanaggacatgaaactgatgagaatagagcgaaggttgagataacgcccaattcaccaagagccgaagccaccctgggaggttttggaagcctaggacttgattgtgaaccgaggacaggccttgacttttcagatttgtattacttgactatgaataacaagcattggttggtgcacaaggagtggttccatgacattccactaccttggcatgctggggcagacaccggaactccacattggaacaacaaagaagcattggtagagttcaaggacgcacatgccaaaaggcaaactgtcgtggttctagggagtcaagaaggagccgttcacacggctcttgctggagccctggaggctgagatggatggtgcaaagggaaggctgtcctctggccacttgaaatgtcgcttgaaaatggacaaacttagattgaagggcgtgtcatactccttatgtaccgcggcgttcacattcaccaagatcccggctgaaacgctgcatgggacagtcacagtggaggtacagtatgcagggacagatggaccctgcaaggttccagctcagatggcggtggatatgcaaactctgaccccagttgggaggttgataaccgctaaccctgtgatcactgaaagcactgagaattcaaagatgatgttggaacttgacccaccatttggggattcttacattgtcataggagttggggataagaagatcacccaccactggnacaggagtggcagcaccatcggaaaagcatttgaagccactgtgagaggcgccaagagaatggcagtcttgggagacacagcctgggactttggatcagtcggaggtgctctcaactcattgggcaagggcatccatcaaatttttggagcagctttcaaatcattgtttggaggaatgtcctggttctcacaaatcctcataggaacgttgctggtgtggttgggtctgaacacaaagaatggatctatttcccttacgtgcttggccttagggggagtgttgatcttcctatctacagccgtctctgctgatgtggggtgttcggtggacttctcaaagaaggaaacgagatgcggtacgggggtgttcgtctataacgacgttgaagcctggagggacaggtacaagtaccatcctgactcccctcgtagattggcagcagcagtcaagcaggcctgggaagatgggatctgtgggatctcctctgtttcaagaatggaaaacattatgtggagatcagtagaaggggagctcaacgcaattctggaagagaatggagttcaactgacggtcgttgtgggatctgtaaaaaaccccatgtggagaggtccgcagaggttgcctgtgcctgtgaatgagctgccccacggttggaaggcctgggggaaatcgtactttgtcagggcagcaaagaccaacaacagctttgttgtggatggtgacacactgaaggaatgcccgctcaaacacagagcatggaacagctttcttgtggaggatcacgggttcggggtatttcacactagtgtctggcttaaagtcagagaggattactcattagagtgtgatccagccgtcataggaacagctgctaagggaaaggaggccgtgcacagtgatctaggctactggattgagagtgaaaagaacgacacatggaggctgaagagggctcacctgatcgagatgaaaacatgtgaatggccaaagtcccacacactgtggacagatggaatagaagaaagtgatctgatcatacctaagtctttagctgggccactcagccaccacaacaccagagagggctacaggactcaagtgaaagggccgtggcatagtgaagagcttgaaatccggtttgaggaatgtccaggcaccaaggtccacgtggaggaaacatgtggaacgagaggaccgtccctgagatcaaccactgcaagcggaagggtgatcgaggaatggtgctgcagggaatgcacaatgcccccattgtcgttccgggcaaaagatggctgttggtatggaatggagataaggcccaggaaggaaccagagagtaacctagtaaggtcaatggtgactgcaggatcaactgatcacatggatcacttctcccttggagtgcttgtgattctgctcatggtgcaggaagggctgaagaagagaatgaccacaaagatcatcataagcacatcaatggcagtgttggtagctatgatcctgggaggattttcaatgagtgacttggctaagcttgcaattctgatgggtgccaccttcgcggaaatgaacactggaggagatgtagctcatctggcgctgatagcggcattcaaagtcagacccgcgttgctggtctctttcatcttcagagccaattggacaccccgtgagagcatgctgctggccttggcctcgtgccttctgcaaactgngatctccgccctggaaggcgacctgatggttctcatcaatggttttgctttggcctggttggcaatacgagcgatggctgttccacgcactgacaacatcaccttggcaatcctggctgctctgacaccactggcccgaggcacactgcttgtagcgtggagagcaggccttgctacttgtggggggttcatgctcctctctctgaaggggaaaggtagtgtgaagaagaacctaccatttgtcatggccttgggactaaccgctgtgaggctggttgaccccatcaacgtggtgggactgctgttgctcacaaggagtgggaagcggagctggccccctagtgaagtactcacagctgttggcctgatatgtgcactggccggagggttcgccaaagcagatatagagatggctgggcccatggctgcagttggcctgctaattgttagttacgtggtctcaggaaagagtgtggacatgtacattgaaagagcaggtgacatcacatgggaaaaagatgcggaagttactggaaacagcccccggctcgatgtggcactagatgagagtggtgatttctccctggtggaggatgatggtccccccatgagagagatcatactcaaggtggtcctgatgaccatctgtggcatgaacccaatagccataccctttgcagctggagcgtggtatgtgtatgtgaagactggaaagaggagtggtgctctatgggatgtgcctgctcccaaggaagtaaaaaagggggagaccacagatggagtgtatagagtgatgactcgcagactgctaggttcaacacaagttggagtgggagtcatgcaagagggggtcttccacactatgtggcacgtcacaaaaggatccgcgctgaggagcggtgaagggagacttgatccatactggggagatgttaagcaggatctggtgtcatactgtggcccgtggaagctagatgccgcttgggacggacacagcgaggtgcagcttttggccgtgccccccggagagagagcgaggaacatccagactctgcccggaatattcaagacaaaggatggggacatcggagcagttgctctggactacccagcaggaacttcaggatctccgatcctagacaagtgtgggagagtgataggactctatggcaatggggtcgtgatcaaaaatggaagttatgttagtgccatcacccaagggaggagggaggaagagactcctgttgaatgcttcgaaccttcgatgctgaagaagaagcagctaactgtcttggatctgcatcctggagctgggaaaaccaggagagttcttcctgaaatagtccgtgaagccataaaaacaagactccgcacggtgatcctggctccaaccagggttgtcgctgctgaaatggaggaagcccttagagggcttccagtgcgttacatgacaacagcagttaatgtcacccactctgggacagaaatcgttgatttaatgtgccatgccaccttcacttcacgcctactacaacccattagagtccccaactacaatctttacattatggatgaggcccacttcacagatccctcaagtatagcagcaagaggatacatatcaacaagggttgagatgggcgaggcggctgccatcttcatgaccgccacaccaccaggaacccgcgacgcatttccggactctaactcaccaatcatggacacagaagtggaagtcccagagagagcctggagctcaggctttgattgggtgacggatcattctggaaaaacagtttggtttgttccaagcgtgaggaacggcaacgagatcgcggcttgtctgacaaaagctggaaaacgggtcatacagctcagcagaaagacttttgagacagagttccagaaaacaaaaaatcaagagtgggacttcgtcgtaacaactgacatctcagagatgggcgccaacttcaaagctgaccgggtcatagattccaggagatgcctgaagccggtcatacttgatggcgagagagtcattctggctggacccatgcctgtcacacatgccagcgctgcccagaggagggggcgcataggcaggaatcccaacaaacctggagatgagtatatgtatggaggtgggtgcgcagagactgatgaagaccatgcacactggcttgaagcaagaatgcttcttgataacatttacctccaagatggcctcatagcctcgctctatcgacctgaggccgataaggtagcagccattgagggagagttcaagcttaggacggagcaaaggaagacctttgtggaactcatgaaaagaggagatcttcctgtttggctggcctatcaggttgcatctgccggaataacctacacagatagaagatggtgttttgatggcacgaccaacaacaccataatggaagacagtgtgccggcagaggtgtggaccagatacggagagaaaagagtgctcaaaccgaggtggatggacgccagagtttgttcagatcatgcggccctgaagtcattcaaagaatttgccgctgggaaaagaggagcggcctttggagtgatggaagccctgggaacactgccaggacacatgacagagaggtttcaggaagccattgacaacctcgctgtgctcatgcgggcagagactggaagcaggccctacaaagccgcggcggcccaattaccggagaccttagagaccatcatgcttttgggtttgctgggaacagtctcgctgggaatcttctttgtcttgatgcggaacaagggcatagggaagatgggctttggaatggtgacccttggggccagtgcatggcttatgtggctctcggaaattgagccagccagaattgcatgtgtcctcattgtcgtgtttctattgctggtggtgctcatacctgagccagaaaagcagagatctccccaggacaaccaaatggcaattatcatcatggtagcagtgggtcttctgggcttgataaccgccaatgaactcggatggttggagagaacaaaaagtgacctaggccatctaatgggaaggagagaggagggggcaaccatgggattctcaatggacattgacttgcggccagcctcagcttgggctatctatgccgctctgacaactctcatcaccccagccgtccaacatgcggtaaccacttcatacaacaactactccttaatggcgatggccacgcaagccggagtgttgtttggcatgggcaaagggatgccattctatgcgtgggacttcggagtcccgctgctaatgatgggttgctactcacaattaacacccttgaccttaatagtggccatcattctgctcgtggcgcactacatgtacttgatcccaggtctacaggcagcagcggcgcgcgctgcccagaagagaacggcagctggcatcatgaagaaccctgttgtggatggaatagtggtgactgacattgacacaatgacaattgacccccaagtggagaaaaagatgggacaagtgctactcatagcagtagccatctccagtgccgttctgctgcgcaccgcctgggggtggggggaggctggggccctgatcacagccgcaacttccactttgtgggaaggctctccgaataaatactggaactcctccacagccacttcactgtgtaacatttttaggggaagttacttggctggagcttctcttatttacacagtaacaagaaacgctggcctggtcaagagacgtggaggtggaacgggagagaccctgggggagaaatggaaggcccgcctgaaccagatgtcggccctggagttttactcctacaaaaagtcaggcatcaccgaagtgtgcagagaagaagcccgccgcgccctcaaggacggagtggcaacaggaggccatgctgtgtcccgaggaagcgcaaagcttagatggttggtggagagaggatacctgcagccctatggaaaggtcattgatcttggatgtggcagagggggctggagttactacgccgccaccatccgcaaagttcaagaggtgaaaggatacacaaagggaggccctggtcatgaagaacccacgttggtgcaaagctatggatggaacatagtccgtcttaagagtggggtggacgtctttcacatggcggcggagtcgtgtgacactttgctgtgtgacataggtgagtcatcatctagtcctgaagtggaagaagcacggacgctcagagtactctccatggtgggggattggcttgaaaaaagaccaggggccttttgtataaaggtgttgtgcccatacaccagcaccatgatggaaaccctagagcgactgcagcgtaggtatgggggaggactggtcagagtgccactctcccgcaactctacacatgagatgtactgggtctctggagcgaaaagcaacatcataaaaagtgtgtccaccacgagccagctcctcttgggacgcatggacgggcccaggaggccagtgaaatatgaggaggatgtgaatctcggctccggcacgcgagctgtggcaagctgcgccgaagctcccaacctgaagatcattggtaaccgcgttgagaggatccgcagtgagcatgcggaaacgtggttctttgatgagaaccacccatacaggacatgggcttaccatgggagctacgaggcccctacacaagggtcagcgtcttctctcataaacggggttgtcaggctcctgtcaaagccctgggatgtggtgactggagtcacaggaatagccatgaccgacaccacaccgtatggccagcaaagagttttcaaggaaaaagtggacactagggtgccagacccccaggaaggcactcgtcaggtgatgaacatggtctcttcctggctatggaaggagctaggtaaacacaaacggccacgagtttgcaccaaagaagagttcatcaataaggttcgcagcaatgcagcactgggggcaatatttgaagaggagaaagaatggaagactgcagtggaagctgtgaacgatccaaggttctgggccctagtggacaaggaaagagagcaccacttgagaggagagtgtcagagctgtgtgtacaacatgatgggaaaaagagaaaagaagcaaggggaatttggaaaggccaagggcagccgcgccatttggtacatgtggctaggggctagatttctagagtttgaagcccttggattcttgaacgaggatcactggatggggagagagaattcaggaggtggtgttgaagggctgggattacaaagacttggatatgttctagaagaaatgagccgcacaccaggaggaaagatgtatgcagatgataccgctggctgggacacccgcatcagtaggtttgatctggagaatgaagctctgatcaccaaccaaatggagaaagggcacagggccttggcgttggccataatcaagtacacataccaaaacaaagtggtaaaggtccttagaccagctgaaagagggaagacagttatggacatcatctcaagacaagaccaaagagggagcggacaagttgttacttacgctcttaatacattcaccaacctggtggtgcagctcattcggaacatggaggctgaggaagttctagagatgcaagacttgtggctgttgaggaggccagagaaggtgaccagctggttgcagagcaacggatgggataggctcaaacgaatggcagtcagtggagatgattgtgttgtgaaaccaattgatgataggtttgcacatgccctcaggtttttgaatgacatggggaaagttaggaaggacacacaggagtggaaaccctcaactggatggagcaactgggaagaagttccgttttgctcccatcacttcaacaagctttacctcaaggacgggaggtccattgtggtcccctgtcgccaccaagatgaactgattggccgagcccgcgtctcaccaggggcgggatggagcatccgggagactgcttgcctagcaaaatcatatgcacaaatgtggcagcttctttatttccacagaagggacctccgactgatggccaacgccatttgttcatctgtgccagttgactgggttccaactgggagaaccacctggtcaatccatggaaagggagaatggatgaccactgaggacatgcttgtggtgtggaacagagtgtggattgaggagaacgaccacatggaggacaagaccccagtcacgaaatggacagacattccctatttgggaaaaagggaagacttatggtgtggatctcttatagggcacagaccacgcactacttgggctgagaacattaaagacacagtcaacatggtgcgcaggatcataggtgatgaagaaaagtacatggactacctatccactcaagttcgctacttgggtgaagaagggtccacacctggagtgtta

SEQ ID NO: 13 - DNA, 10290 bp, Zika virus
atgaaaaacccaaaaagagccggtagcagccggcttgtcaatatgctaagacgcggtgcagcccgtgtcatccctccaggaggagggctcaagaggctgcctgtaggattgctgttgggtcggggtccgatcaaaatgatcctggccatactggcattcctacgatttacagcaataaaaccgtccactggcctcatcaacagatggggaaaagtgggcaaaaaagaggccatcaaaatcctcacaaaattcaaggctgacgtgggcaccatgctgcgtatcatcaacaatcggaagacaaaaaagagaggagtcgaaactggaattgtgttcctggcattgctggtgtctattgttgctgtggaagtcacaaaaaagggggacacctattacatgtttgcggacaagaaggacgccggaaaggtggtgacctttgagactgaatctggacccaaccgttgctccatccaagcaatggacattggacatatgtgtccagctacaatgagctatgaatgtcccgtgctggaaccacagtatgagccagaggatgtcgactgttggtgcaactcgacagcagcatggattgtgtatggcacatgcacccacaagacaacgggagagacaagacgttccagacgttcaatcaccctgccatctcatgcctcacaaaagttggagaccagatcatcgacgtggcttgaatcccgcgaatactccaaatatctaataaaggtggaaaactggatcctccgcaatccaggatatgcgttggtggctgcagtgattggatggactctgggcagcagtcgcagccagaagatcatctttgtcactctgctcatgttggtagcccccgcatacagcatcagatgcattggaattggaaacagagacttcattgagggaatgtccggtggcacctgggtggacattgtcctggaacatggtggttgtgtgacagtaatgtcaaacgacaaacccacattggactttgaactggtgacaacgaccgcaagtaacatggctgaggtcaggtcctactgctatgaagctaacatatccgagatggcatcggacagcaggtgccccacacagggggaagcttatcttgacaaaatggccgactcccagtttgtgtgcaagcgtgggtacgttgacaggggctggggaaacggatgtggactctttggaaaaggaagcattgtcacttgcgctaagttcacgtgtgtgaaaaagctcacagggaaaagcattcaaccggagaatctcgagtaccgggtccttgtttcggtgcacgcttcccaacatggaggaatgattaacaatgacaccaatcaccaacacgacaaggagaacagagcgcgcattgatatcacagctagcgctccccgtgttgaggtggaacttggctcctttggatccttctcgatggagtgtgaaccccggtcaggattgaactttggtgacctgtattacctcaccatgaacaacaagcattggctggttaatagagattggtttcacgatctttccttgccatggcatacaggagccacatcaaacaatcatcactggaacaacaaggaggcgctggtagaattcagagaagcccacgcaaagaagcagacggctgtggtcctgggaagtcaggaaggagctgttcacgcagcactggccggcgcactggaggctgagtctgatggacacaaagcgactatctactctggacacttgaagtgtcgcttgaagctagacaaactgcgcctgaagggaatgtcatatgcactctgcacaggagcattcaccttcgctcgcaccccctctgaaacaattcacggcaccgccacagtggagctgcaatatgcaggtgaagatgggccgtgcaaagttcccatagtaattaccagtgacaccaatagcatggcctcgacaggcaggctgatcacagcgaatccggtggtcacggaaagtggagcaaactcaaagatgatggtcgagattgaccctccgtttggtgattcttacattattgtgggcactggcacaacaaaaattacccaccattggcacagagccggtagttcaattggacgtgcatttgaggctaccatgagaggagcaaaacggatggcggtcctcggcgacaccgcttgggactttggctctgttgggggcatgttcaactccgttggaaagtttgtccaccaggtgtttggatcagcatttaaggcattgtttggaggcatgtcctggttcacacagctcctgataggatttctgctcatatggatgggtttgaacgcacgcggtggaaccgtggccatgagcttcatgggcattggggctatgctgattttcctagccacctcggtgtcaggagacacaggatgctcggttgacatatccagaagggaaatgcggtgcgggagcggcatattcgtgtacaatgacgttgacgcatggcgaagccgctacaaataccatcctgaaacccccagagctttggccgctgccgtgaaaacggcttgggaagaagggacctgtggcattacctcagtgagcagaatggaaaacctgatgtggagctctgtggctggagagttgaatgcaatccttgaggacaattcagtgccattgacagtcgtcgttggcgagccaaaatatccactgtacaatgctccaaagaggctgaaaccaccagcatcagagttaccgcaggggtggaagtcctggggaaagtcatactttgtctcagccgcaaaaaacaacaactcctttgtggtagatggtgacaccatgaaggaatgcccaagacagaagcgagcatggaacagcttgagaatagaggatcatgggttcggagtcttccacactagcatctggctgaaattccatgaggacaactccaccgaatgtgacacagctatcataggaacggcggttcgcgggaaggaagccgttcatagtgacttgggctactggatagagagtgagcgcaatgacacatggaggctctctcgagcgcacctgatcgaagcaaagacatgtgaatggccacggtcgcacacactgtggacggacggagtggaagagagcgagctgatcattccacgtggcttagccggtcctttcagccatcataacacgcgtgctggctacaagactcagaataaaggtccctggcatttaggtgatgttgaaattcagttcgccacgtgccccggaacaaccgtggtccaggaccaagagtgcagggacaggggcgcttctctacgcacgaccacagctagtggaagggtaatcaatgaatggtgctgcaggtcgtgcaccatgcctccactcagtttcaagacaaaagatggatgttggtatgcaatggagatacgtcctgtgaaagaacaagagtcaaacctcgtgcgatcgcacgtcactgccggaagcacagaccacatggaccatttctctctcggattagtagtggtcatgttgatggtgcaagaaggtatgaagaagagaatgacatcaaaagcaataatcacctcagcggcctttctcctggcggttatgatagtgggaggtttcacgtaccaggattttgggaggctggtggtattggtgggtgctgcatttgctgagatgaacactggaggtgacgttgcgcacctggcgctggtggcagcgtttaaagtgaggccagcgatgctggtctcattcatgttcagagccttgtggacccccagggagtcactgcttttagctctggctgcctgcctcctgcaggtgtcagtgacaccactggatcattccatcatgatcgtggttgatgggattgcgctgtcctggttgtgtctgaaagccatcttggtgccgcgtaccccaaacatagcccttcctcttctcgctatgctgtcacccatgctccaaggtaccaccattgtggcatggcgagctatgatggcggccctggctgtcataaccttggcttccatgaagcatggaaggggtgtaaaaaagacgtttccctacaccatcggatgcatccttggcagcatgggcttagttgaaaacttggggttggttggcctcctcttgttgacagcctcaaaaaagaggagttggcctccgagtgaggtgatgacggctgtcggactgatctgtgcaattgtgggcggactaaccaagaccgacattgacatggcgggacccatggcagccataggactgctggtggtgagctatgtggtttctggcaagagtgtggacatgtacattgaaaaggtgtgtgacatatcatgggacaaggacgctgaaataacaggcacaagtccgcggctggatgtggctctcgacgacagtggagatttctcacttatccaggatgacgggccccccactcgagagattgtgttgaaggtgtttctgatgtgtgtttgcggtgtcagccccatagccatcccctttgcagccgctgcttggttcgtgtacattaaatcagggaaaagaagcggcgccatgtgggacattccatccccaagagaagtgaaaaaaggggaaacaacggctggagtgtacagaatcatgacgcgtaaattgctgggcagcacacaggtgggagccggagtaatgcatgaaggtgtttttcacacaatgtggcacgtcacaaaaggttcggcccttcggagtggtgagggacgcctagatccatactggggaaacgtgaagcaggatttgatctcttactgcggaccatggaaactggatgggaaatgggacggcgtgtcggaagtccaactgatagcggtcgccccaggtgagcgcgccagaaatgtgcagacaaaaccaggagtgttcaagaccactgatggggaaatcggggccttggcccttgacttcccaggcggaagttcaggctccccgataattgacaaaaatggacatgtaattggcctgtatggaaatggtgtcgtggtcaagagtggaagctacgtgagtgccatcatgcagacagagaagatggaggaacccgcagttgactgctttgaggaggacatgctgagaaaaaagaagctgacggtgctcgacctccatccaggagctggaaaaactcgaagagtgctccctcagatcgtcaaggctgcaattaagaaacgcctacgcacggtaatcctggcacccacccgagtggtggcagctgagatggctgaggcactaaaagaccttccaataaggtacatgactccggcagtttcagccacccatgatggcaatgagattgttgaccttatgtgccacgccacttttacatcaaggctaatgcaaccaattagggtgcctaattacaatctatatataatggatgaggcccacttcacagatcctgcaagcatcgctgcaagagggtacatagcaacaagagtggacatgggagacgccgcggccatcttcatgacggccacccctcctggcagcactgaagctttcccggattcaaacgcccccatcacagatgttgaaacagaggttcctgacaaggcgtggaattctggatttgaatggatcactgattacccagggaaaaccgtttggtttgtccctagtgtcagaatgggcaatgagatctcggcctgcctcacaaaagccggcaaatcggttatccaactcagccggaaaacctttgaaacagagtaccagaagacaaagaatggtgagtgggactttgtcgtgaccactgacatctcagaaatgggagccaacttcaaggccgacagagtcatagactcacggaaatgcttgaagccagtgattctggatgacatggaagagagagttgttcttgccgggccgatggcagtaacaccatccagcgcagctcaacgcagaggaagaattggaagaaaccccaacaaaactggagatgagttctattacggggggggctgtgccgcaacggatgatgaccatgctcattgggtagaggctaggatgctgcttgacaacatctacctccaggacaacctcgttgcatctctgtacaagccagaacaaggaaaggtctcggcaatagaaggggagttcaaactgagaggagaacagaggaaaaccttcgtggagctgatgaagagaggggacttgccagtgtggttgtcatatcaagtggcggcctccggactcagctatactgaccggcgctggtgctttgatggaaaaaacaacaacaccatcctggaggactgcgtccccgtcgaggtgtggacaaaatttggagagaaaaagattctgaagcccagatggatggacgctcggatctgctctgatcatgcctctttgaagtctttcaaggagtttgctgcaggaaagagaacaatagccactggcttaattgaggcttttgggatgcttcccgggcacatgactgagagattccaggaggccgtcgacaatttggccgtgttgatgagggccgaggcaggctctagggcacacagaatggctgcagcacagctccctgagacaatggaaaccatcctgctcctcagcctgctggcattcgtgtcacttggtgtattttttgtactgatgagggcaaaagggttaggaaaaatggggtccggcatgatcgtgctggcaggaagtggctggctcatgtggatgtctgaggtggaaccagcccgcatagcttgtgtggtgatcatagtgtttctgctaatggtcgttctgattccggaaccggagaagcagcgctctccccaggacaatcagctggctctaattatcttgatcgcgacgggcctcatcacgctcatcgcggccaatgagctgggttggttagaaagaacaaagagtgacctcaccaggctgttttggagagaacacgctgagccaacaggagggagagggttttccttctcgctggacattgacctgcggccggcatcggcctgggcaatatatgccgctatgacaaccctgatcacaccgacagtccaacacgctgtgaccacatcgtacaacaactactctctcatggctatggccactcaggccggagttctttttggcatgggacggggggtgcctttttacaaatgggactttggcgtgccactccttatgctgggctgctactcacaacttaccccactcaccctgatcgtggctctcgtgatgctagccgctcactatctctatctcatccccgggctccaggcaacggccgccagggccgcccaacgaaggacggctgctggaataatgaaaaacccagtggtggatggaattgtggtaactgacatagacccaatccaaatcgatccaaatgtcgaaaagaagatgggccaggtcatgctcatctttgtggctttggcgagcgcggttctcatgagaacggcatggggttggggagaggctggtgcccttgcatcggcagcagctgccaccctatgggaaggggctcccaacaagtactggaattcatcaacggctacatccttgtgcaacatatttcggggaagttatctggcaggtccctccctcatctacaccgtcacacgcaatgcaggtatcatgaagaaaaggggcggtggaaatggagaaacggtgggcgagaaatggaaggagcgcttgaatcggatgaccgcgcttgaattctacgcctacaagcggtcaggaataactgaagtgtgcagagaacccgccagaagagccttgaaggatggagtcgtcacaggaggacacgctgtctcccgcggaagcgcaaagctgcgatggatggtggaacgtggccacgtcaatctagtgggacgcgttgtcgacctcggatgtggaaggggtggctggagttactacgccgcatctcaaaagcaagtcctcgaggtgagaggctacacaaaagggggagcgggccacgaggagcccatgaatgtccaaagttatggttggaacatagtgcgactcaagagtggagtggacgttttttatctaccatcagaaccatgtgacacgctgctctgtgacattggagagtcatcctcgagcccagcagtggaagaagcccggactctgagagtgctcgggatggttgaaacctggctggaacggggcgtaaagaacttctgcatcaaagtgctctgcccgtacaccagtgccatgattgagcggctggaagccctccagcgtcgctacggaggaggcctggtgagggttccactctccagaaattccacccacgaaatgtactgggtctctggagcaaaatcaaacatcatcaggagtgtgaatgccaccagccagctgctcatgcacagaatggacatccccacgcggaaaacaaagtttgaagaagacgtcaatctggggaccggaaccagggcagttgaaagcagagctgaccctcccgacatgaaaaaactaggcagccggattgagcggttgagaaaggaatatggatccacttggcactacgatgaaaaccacccctacaggacatggcattaccacggcagttatgaggctgacacgcaaggctccgcctcctcaatggtcaacggcgtggtgcgtctcctctcaaaaccatgggatgcattgagctcggtcaccaacattgctatgacggacacaactccgtttggacagcagcgggtgttcaaggagaaagtggacacccggactccagaccccaagcagggcacgcaaagagtcatggccataacatcacaatggctgtgggaccgcctagcaagaaacaagacccctcggatgtgcacgcgacaggaattcataaacaaggtcaacagtcacgcggcgttgggacccgtttttagagaacagcagggatggggttcagcggccgaagcggtggtagatcctaggttttgggagctcgttgacaatgaaagagaagcccatttgagaggggagtgcttgacctgtgtctacaacatgatggggaaaagagaaaagaagctcggtgaattcgggaaggcaaaaggcagcagagccatttggtacatgtggctgggagcccgcttcctcgagttcgaggccctgggcttcctcaatgaagaccactggttaagcagagagaactctggagggggagttgagggcttgggcctccaaaaacttggatacatccttgaagagatcagcaggaggccaggaggcaaaatgtatgccgatgacacggctggctgggacacccgcatcacgaaatgcgacctagaaaatgaggcgcgcattttggaaaaaatggacgggatccacaaaaaactcgcacgggccgtcatcgagttgacatacaagcataaggttgtgagagtcttgagaccagcaccacaagggaaggtcgttatggacatcatctccaggccagaccaaagggggagtgggcaggtggttacttatgccctcaacacctatacaaacttggtggtgcagctgatccgtaacatggaagcagaggctgtcatcaatgaaagagacatggaggagctccaaaacccatggaaagtcatcaattggctagaaggaaatggatgggacagactccgctcgatggcagtgagtggagatgactgtgtcgtgaaaccaatggatgataggttcgcctatgcactgaatttcctcaatgacatgggcaaggtcagaaaagatgtccaggaatggaagccctcgccggggtggacaaactgggaagaagtgcccttttgctcccaccacttcaacaagctcccgatgaaggatggaagaacaataatagttccctgccggcaccaagatgagttgataggcagggctagagtttctccaggaaaaggctggtcactcagtgaaacagcatgcttgggcaagtcttatgcccagatgtggctactgttgtactttcacaggagagatctccgactcatggcaaacgcaatctgctctgctgtaccggtgagttgggtgcccacggggagaacaacctggtccatccatgggcgtggagagtggatgacaacagaggacatgctagaggtatggaacagagtgtggatcatagagaatgagtacatggaggacaagacccctgtcacagagtggaccgatgttccatacttgggaaagagagaagacttgtggtgcggctcccttattggacacaggccaagaagcacatgggcagagaacatctgggctgccatttatcaagtgcgccgagcaatcggcgaaactgaagaatatagagactacatgagcacacaggtccgctatggctcggaggaagggccaagcgctggtgtgttgtaa

SEQ ID NO: 14 - DNA, 4251 bp, Artificial Sequence
  A synthetic oligonucleotide
  misc_feature (1)...(4251): n = A,T,C or G
acgaccgaagcgcancgagntcagtgagcgaggaagcggaagagcgcccaannnnnnnnnnnnnnnnnnnnnnnnnnnnnnnnattcattaatgcagaattccttccgagtgagagacacaaaaaattccaacacactattgcaatgaaaannnnnnnnnnnnnnnnnnnnnnnnnnnnnnnntcaaggggcttcatgatgtccccataatttttggcagagggaaaaagatcatacgtagatcgaaacgannnnnnnnnnnnnnnnnnnnnnnnnnnnnnnncagttcctatgagctttgctccagaccggctgcgagcaaaacaagcggctaggagttccgcagtatggnnnnnnnnnnnnnnnnnnnnnnnnnnnnnnnnccgctgatggcctatggccaagccccagccagtgggggttgcgtcagcaaacacttggcacagaccagnnnnnnnnnnnnnnnnnnnnnnnnnnnnnnnntactgtttacttagaaaggccttgtaagttggcgagaaagtgaaagcctgtttagcttgtatacatgcnnnnnnnnnnnnnnnnnnnnnnnnnnnnnnnnctgtaaatggagcagcaaagcccaaaagacccacaatcctttgacatactttccaatcaataggcctgnnnnnnnnnnnnnnnnnnnnnnnnnnnnnnnncttttgtaacctatgatcttgtggcaatgtcccccaacttccaattacgtaacccatgaagtttagggnnnnnnnnnnnnnnnnnnnnnnnnnnnnnnnnagatcctcacaccgagcaccccacatcagcagagacagctgtggataagaagatcaacactccccctannnnnnnnnnnnnnnnnnnnnnnnnnnnnnnntttgtgttcagacccaaccacatcagcaacgttccaatgagaatttgtgagaaccaggacattcctccnnnnnnnnnnnnnnnnnnnnnnnnnnnnnnnncatggatgcccttgcccaatgagttgagagcgcctccaacttagccaaagtcccaggctgtgtctcccnnnnnnnnnnnnnnnnnnnnnnnnnnnnnnnncgcttcaaatgcttttccaatggtgcgtccactcctgtgccagtggtgggtgatcttcttctccccgannnnnnnnnnnnnnnnnnnnnnnnnnnnnnnncgatcaagttccagcatcatcttagagttctcagtgctttcagtgattacggggttagcggttatcaannnnnnnnnnnnnnnnnnnnnnnnnnnnnnnnccgccatctgacgtggaaccttgcaaggtccatctgtccctgcgtactgtacctccactgtcactgtcnnnnnnnnnnnnnnnnnnnnnnnnnnnnnnnntgtgaacgctgcggtacacaaggagtatgacacgcccttcaatctaagtttatccattttcaggcgacnnnnnnnnnnnnnnnnnnnnnnnnnnnnnnnnccaccatccatctcagcctccagagctccagcaagggccgtgtgaactgctccttcttgactccctagnnnnnnnnnnnnnnnnnnnnnnnnnnnnnnnnccttgaactctaccagtgcttctttgttgttccagtgtggagttccggtgtctgccccagcgtgccaannnnnnnnnnnnnnnnnnnnnnnnnnnnnnnnaaccaaccagtgcttgttattcatagtcaagtaatacaaattcgaaaagtcaaggcctgtcctcggttnnnnnnnnnnnnnnnnnnnnnnnnnnnnnnnnagggtggcttcggctcttggtgaattgggcgttatctcaaccttcgctctattctcatcagtttcatgnnnnnnnnnnnnnnnnnnnnnnnnnnnnnnnncggagccatgaactgacagcattatccggtactccagattctctggctggatgctcttcccggtcattnnnnnnnnnnnnnnnnnnnnnnnnnnnnnnnncaggctccctttgccaaaaagtccacatccatttccccagcctctgtccactaagcttcttttgcagannnnnnnnnnnnnnnnnnnnnnnnnnnnnnnnccttcaccttgtgttgggcagcggctgtccgaagccatgtcctagatttaggcctcatagcagtaggannnnnnnnnnnnnnnnnnnnnnnnnnnnnnnntaaccagctctatgtcgacagtcggtttgtccgtggccattacggtgacacaacctccatgttccaagnnnnnnnnnnnnnnnnnnnnnnnnnnnnnnnnttccacaaagtccctattgctgactcctatgcacctgatgctgtagtccggggcaatcagcagtatcannnnnnnnnnnnnnnnnnnnnnnnnnnnnnnncttcccaaaagccaagcgatggcagctgctgctaacgcgaagccagggttcctgaatatccaattttcnnnnnnnnnnnnnnnnnnnnnnnnnnnnnnnnattccaaccaggtttgcgaccgcgtttgcagcttcctagtggaatgggaggggagcgtcacagctcttnnnnnnnnnnnnnnnnnnnnnnnnnnnnnnnnccaggttccgtacacaacccaagttgacgtcgtgttgcaccaacaatcgacgtcatctggttccacccnnnnnnnnnnnnnnnnnnnnnnnnnnnnnnnnctggcatcacacatgtgtccaagatccatgatctgtatataacacttattcatccccaatgtggttggnnnnnnnnnnnnnnnnnnnnnnnnnnnnnnnnccaagtacatatagtatgcactcccacgtctagtgacctccgctgccatagctgtggtcagcaggaggnnnnnnnnnnnnnnnnnnnnnnnnnnnnnnnnagggtccccagtcctcgcggagattgacgagatgtgagaggcaatattcggagcagggtttactgttcnnnnnnnnnnnnnnnnnnnnnnnnnnnnnnnncccctgactctgggatcctgacctggaagatagtcagggttgaggcaagcaaaaggtacatgtaagagnnnnnnnnnnnnnnnnnnnnnnnnnnnnnnnntcttgactggggaagccaggcccaccctggagagtacatacctgcttgctgagatccggacggtgagtnnnnnnnnnnnnnnnnnnnnnnnnnnnnnnnncaccgttcccggccgcggaggctggatcggtcccggtgtcttctatggaggtcaaaacagcgtggatgnnnnnnnnnnnnnnnnnnnnnnnnnnnnnnnncagctctgcttatatagacttcccaccgtacacgcctaccgcccatttgcgtcaacggggcggggttannnnnnnnnnnnnnnnnnnnnnnnnnnnnnnnctgccaaaacaaactcccattgacgtcaatggggtggagacttggaaatccccgtgagtcaaaccgctnnnnnnnnnnnnnnnnnnnnnnnnnnnnnnnnatcaccatggtaatagcgatgactaatacgtagatgtactgccaagtaggaaagtcccgtaaggtcatnnnnnnnnnnnnnnnnnnnnnnnnnnnnnnnncgtcattgacgtcaatagggggcggacttggcatatgatacacttgatgtactgccaagtgggcagttnnnnnnnnnnnnnnnnnnnnnnnnnnnnnnnncaaagtccctattggcgttactatgggaacatacgtcattattgacgtcaatgggcgggggtcgttggnnnnnnnnnnnnnnnnnnnnnnnnnnnnnnnntatgtaacgcggaactccatatatgggctatgaactaatgacccctgaattgattactattaataactnnnnnnnnnnnnnnnnnnnnnnnnnnnnnnnnccctgacatcgcgaagcagcgcaaaacgcctaaccctaagcagattcttcatgcaattgtcggtcaagnnnnnnnnnnnnnnnnnnnnnnnnnnnnnnnnccactactcagcgacctccaacacacaagcagggagcagatactggcttaactatgcggcatcagagcnnnnnnnnnnnnnnnnnnnnnnnnnnnnnnnncgagatctcccgatccgncgacgtcaggtggcacttttcggggnaatgngcggggaacccnnanttntnnnnnnnnnnnnnnnnnnnnnnnnnnnnnnnnngcgtcatgagacaataaccctgataaatgcttcaataatatgaaaaaggaagagtatgagtatcaaa

(19) United States
(12) Patent Application Publication
(10) Pub. No.: US 2018/0028643 A1
(43) Pub. Date: Feb. 1, 2018

(54) ZIKA VIRUS VACCINES USING VIRUS-LIKE PARTICLES

(71) Applicants: Brock Adam Kingstad-Bakke, Madison, WI (US); Jorge E. Osorio, Mount Horeb, WI (US)

(72) Inventors: Brock Adam Kingstad-Bakke, Madison, WI (US); Jorge E. Osorio, Mount Horeb, WI (US)

(21) Appl. No.: 15/629,503

(22) Filed: Jun. 21, 2017

Related U.S. Application Data
(60) Provisional application No. 62/352,904, filed on Jun. 21, 2016.
(60) Provisional application No. 62/384,967, filed on Sep. 8, 2016.

(51) Int. Cl. A61K 39/12; C12N 7/00

(52) U.S. Cl. CPC A61K 39/12; C12N 7/00; A61K 2039/55

(57) ABSTRACT

A flavivirus virus-like particle and methods of making and using that particle, and antibodies raised to a plurality of those particles, are provided.

### CROSS-REFERENCE TO RELATED APPLICATIONS

This application claims the benefit of the filing date of U.S. application Ser. No. 62/352,904, filed on Jun. 21, 2016, and U.S. application Ser. No. 62/384,967, filed on Sep. 8, 2016, the disclosure of which are incorpraoted by reference herein.

### BACKGROUND

Zika virus (ZIKV; Flaviviridae, Flavivirus) is an emerging arbovirus, transmitted by Aedes mosquitoes (loos et al., 2014). ZIKV has a positive-sense, single-stranded RNA genome, approximately 11 kilobases in length that encodes three structural proteins: the capsid (C), premembrane/membrane (prM), and envelope (E), and seven non-structural proteins (NS1, NS2A, NS2B, NS3, NS4A, 2K, NS4B, and NS5). Based on a genetic study using nucleotide sequences derived from the NS5 gene, there are three ZIKV lineages: East African, West African, and Asian (Musso, 2015; Faye et al., 2014). ZIKV emerged out of Africa and previously caused outbreaks of febrile disease in the Yap islands of the Federated states of Micronesia (Duffy et al., 2009), French Polynesia (Cao-Lormeau et al., 2014), and Oceania. Currently, several Latin American countries are experiencing the first-ever reported local transmission of ZIKV in the Americas (Hennessey et al., 2016). The current outbreak in the Americas is cause for great concern, because of the fast and uncontrolled autochthonous spread. Clinically, infection with ZIKV resembles dengue fever and several other arboviral diseases (Dyer, 2015), but it has been linked to neurological syndromes and congenital malformation (Pinto Junior et al., 2015). Alarmingly, the rate of microcephaly (small head, reduced brain size, impaired neurocognitive development) in infants born to pregnant women has increased significantly (20-fold in 2015) in areas with high ZIKV incidence in Brazil (Oliveira Melo et al., 2016) (Butler, 2016). In February 2016, the World Health Organization declared the Zika virus an international public health emergency, prompted by its link to microcephaly. As many as four million people could be infected by the end of the year (Galland, 2016).

To date, there are no vaccines or antiviral therapy for ZIKV, although successful vaccines have been developed for other flavivirus infections (dengue, Japanese encephalitis and yellow fever).

### SUMMARY

Mosquito-borne Zika virus (ZIKV) typically causes a mild and self-limiting illness known as Zika fever, which often is accompanied by maculopapular rash, headache, and myalgia. However, more serious consequences have been reported for ZIKV infection during pregnancy, microcephaly of the fetus. As described herein, Zika virus-like particles (VLPs) were developed and their immunogenicity and protective efficacy were evaluated in a small animal model for wild-type ZIKV. The prM and E genes of ZIKV strain 33 H/PF/2013 with a nascent signal sequence in the 3′ coding region of the capsid protein were cloned into a pCMV expression vector under the control of a cytomegalovirus (CMV) promoter and CMV polyadenylation signal. Following transfection of HEK293 cells, ZIKV-VLPs expression was confirmed by Western blot and transmission electron microscopy. ZIKV-VLPs (about 0.45 μg) were formulated with 0.2% Imject alum and used to inject groups of six-week-old AG129 mice by the intramuscular (IM) route, followed by a boost administration two weeks later. Control groups received PBS mixed with alum. At five weeks post-initial vaccination all animals were challenged with 200 PFU (>400 LD50s) of ZIKV strain H/PF/2013 by injection into the right hind footpad. All control animals (n=6) died 9 days post challenge, while vaccinated mice survived with no morbidity or weight loss and had significantly lower viremia. This was in contrast to Dengue VLPs produced from prM and E, which did not produce a protective immune response (Pijlman, 2015). Significant levels of neutralizing antibodies were observed in all ZIKV-VLP vaccinated mice compared to control groups. The role of neutralizing antibodies in protecting mice was demonstrated by antibody passive transfer studies; naive AG129 mice that received pooled serum from VLP vaccinated animals were fully protected. Thus, the present findings demonstrate the protective efficacy of the ZIKV-VLP vaccine and highlight the role that neutralizing antibodies play in protection against ZIKV infection.

One advantage of VLPs is that VLPs structurally mimic the conformation of native viruses but do not contain any viral genetic material (no viral replication) and are therefore non-infectious. This is in contrast to a live attenuated vaccine (which has genetic material) or in the case of insufficient inactivation of killed vaccines (resulting in viral replication). A VLP vaccine approach eliminates concerns associated with such replication for pregnant women and other populations at high risk for suffering the effects of ZIKV infections.

In one embodiment, a recombinant nucleic acid vector is provided comprising a heterologous promoter operably linked to a sequence encoding flavivirus, e.g., ZIKV, prM/E. In one embodiment, the vector lacks nucleic acid sequences encoding one or more of flavivirus NS1, NS2A, NS2B, NS3, NS4A, NS4B or NS5 and optionally lacks nucleic acid sequences encoding functional flavivirus capsid, e.g., a protein that aggregates so as to form a viral capsid having a diameter of about 50 to 60 nm or about 45 nm to 70 nm. In one embodiment, the heterologous promoter is expressed in mammalian cells. In one embodiment, the heterologous promoter is a heterologous viral promoter. In one embodiment, the heterologous promoter comprises a CMV promoter, a SV40 promoter, an EF-1α promoter or a PGK1 promoter. In one embodiment, the flavivirus is a Zika virus. In one embodiment, the vector sequences are from a Zika virus from the East African or West African lineage. In one embodiment, only a portion of flavivirus capsid sequences is included, e.g., a C-terminal portion of a flavivirus capsid that is linked to prM/E sequences as in the poly-protein that is expressed by wild-type flavivirus. In one embodiment, the portion of the capsid sequence includes amino acids 98 to 112 of the capsid protein encoded by SEQ ID NO:1 or a protein having at least 80%, 82%, 85%, 87%, 90%, 92%, 95%, 97%, 99% or more amino acid sequence identity thereto. In one embodiment, the prM/E sequences have at least 80% %, 82%, 85%, 87%, 90%, 92%, 95%, 97%, 98%, 99% or more amino acid sequence identity to the prM/E sequences encoded by any one of SEQ ID Nos. 1-3, 5 or 11-13. In one embodiment, the portion of the capsid sequence lacks a NS2B-3 cleavage site, e.g., KEKKRR (SEQ ID NO:10). In one embodiment, the prM/E sequences are operably linked to a heterologous secretion signal. In one embodiment, the vector further comprises an intron and/or enhancer sequence, e.g., 5′ to a prM/E coding sequence. In one embodiment, the vector further comprises comprises an intron, internal ribosome entry sequence, or an enhancer sequence, or any combinantion thereof.

A recombinant host cell comprising the vector is also provided. In one embodiment, the cell is a mammalian, e.g., Vero cell, HeLa cell or CHO cell, insect or yeast cell. In one embodiment, the cell is a human or simian cell. In one embodiment, the genome of the cell is augmented, e.g., stably augmented, with nucleic acid sequences encoding flavivirus NS2B, e.g., the source of NS2B may be heterologous or homologous to the source for prM/E. In one embodiment, the genome of the cell is augmented, e.g., stably augmented, with nucleic acid sequences encoding flavivirus capsid, e.g., the capsid may be heterologous or homologous to prM/E, which sequences are optionally integrated into the genome of the cell. In one embodiment, the genome of the cell is augmented with nucleic acid sequences encding flavivuirus NS2B, which sequences are optionally integrated into the genome of the cell. In one embodiment, the vector is integrated into the genome of the host cell.

Also provided is a method to prepare flavivirus VLPs. The method includes contacting a culture of isolated host cells that do not express one or more of flavivirus NS1, NS2A, NS2B, NS3, NS4A, NS4B or NS5 and optionally do not express functional flavivirus capsid, with the recombinant vector and collecting VLPs from supernatant of the culture. Thus, in one embodiment, the isolated host cells do not have flavivirus sequences prior to contact with the vector. In one embodiment, the collected particles have a diameter of about 10 to 100 nm, e.g., 20 to 60 nm, 40 to 70 nm or 40 to 60 nm. In one embodiment, the host cell expresses flavivirus NS2B. In one embodiment, the host cell expresses flavivirus capsid protein and optionally NS2B.

Further provided is a preparation comprising a flavivirus VLPs. The VLP comprises a lipid bilayer comprising flavivinis prM/E but lacks one or more of a flavivirus NS1, NS2A, NS2B, NS3, NS4A, NS4B or NS5 and optionally lacks functional flavivirus capsid. Such a preparation may be used in a vaccine or immunogenic composition. The vaccine or immunogenic composition may have about 10 μg to 1000 μg, e.g., 200 μg to 400 ₗμg or 400 ₗμg to 800 μg, about 0.5 μg to 100 μg, about 1 μg to 50 μg, about 5 μg to 75 μg, about 1 to 500 mg, e.g., about 20 to 50 mg, about 100 to 300 or about 300 to 400 mg, of VLP. The vaccine or immunogenic composition may further comprise one or more adjuvants. In one embodiment, the adjuvant comprises alum, monophosphoryl lipid A (MPLA), squalene, a TLR4 agonist, dimethyldioctadecylammonium, tripalmitoyl-S-glyceryl cysteine, trehalose dibehenate; saponin, MF59, AS03, virosomes, ASO4, CpG, imidazoquinoline, poly I:C, flagellin, or any combination thereof In one embodiment, an adjuvant is included at about 0.001 mg to about 10 mg, about 0.01 to about 10 mg, about 1 to about 20 mg, or about 10 mg to about 100 mg.

Further provided is a method to prevent, inhibit or treat flavivirus infection in a mammal. The method includes administering an effective amount of the recombinant vector, a host cell having the vector or the vaccine or immunogenic composition having the VLPs. In one embodiment, the mammal is a female mammal. In one embodiment, the vector, host cell, vaccine or immunogenic composition is administered subcutaneously, intradermally, intramuscularly or intravenously to the mammal.

In one embodiment, a method to passively prevent, inhibit or treat flavivirus infection in a mammal is provided. The method includes obtaining serum or plasma having anti-flavivirus antibodies from a mammal exposed to flavivinis and optionally isolating antibodies from the serum or plasma; and administering an effective amount of the serum or plasma, or isolated antibodies, to a different mammal at risk of or having a flavivirus infection. In one embodiment, the mammal is immunocompromised. In one embodiment, the anti-flavivirus antibodies are isolated from the serum before administration. In one embodiment, the mammal is a human.

### BRIEF DESCRIPTION OF THE FIGURES

FIGS. 1A-E. In vitro characterization of Zika virus like particles. A) Schematic of pCMV-prM/E expression cassette. B) Western blot analysis of Zika virus like particles. Lanes are, 1) Bio-rad precision plus kaleidoscope protein standards. 2): pCMV-prM/E transfection pre sucrose cushion purification supe. 3) 3.5×10⁴ PFU ZIKV positive control. 4) pCMV-prM/E transfection post sucrose cushion purification pt. 5) pCMV-GFP transfection post sucrose cushion purification pt. C-E) Sucrose cushion purified Zika VLPs observed using transmission electron microscopy. C) VLPs stained with Tungsten. Diameter is indicated. Background protein staining also apparent. D) VLP stained with Tungsten. Membrane proteins visible on the surface of VLP are indicated with arrow. Background protein staining apparent. E) VLP stained with Uranyl acetate. Membrane proteins visible on the surface of VLP are indicated with an arrow.

FIGS. 2A-F. Protection of ZIKVLPS in AG129 mice. A) Neutralizing antibody titers (+/−SD) of vaccinated AG129 mice pre boost and pre challenge. B) Average weight loss (+/−SD) of AG129 after ID challenge with 200 PFU ZIKV over a 14 day period. C) Survival of 11 week old AG129 after ID challenge with 200 PFU ZIKV over a 14 day period. D) Viremia (+/−SD) in serum samples from mice two days post challenge by qRT-PCR. Values are total RNA copies per reaction. E) Viremia (+/−SD) in serum samples from mice two days post challenge by TCID₅₀. F) PRNT₅₀ and PRNT₉₀ values (+/−SD) of serum samples taken from ZIKVLP vaccinated AG129 mice post challenge, and pre challenge serum from PBS/alum mice.

FIGS. 3A-B. ZIKVLP serum transfer to naïve AG129 mice. A) Average weight loss (+/−SD) of 8 week AG129 transferred serum from mice vaccinated with ZIKVLPs after ID challenge with 20 PFU of ZIKV over a 14 day period. B) Survival of AG129 after challenge with ZIKV over a 14 day period.

FIG. 4. LD50 of ZIKV in AG129 mice. Survival of AG129 after ZIKV over a 14 day period.

FIG. 5A-B. A) Weight loss of AG129 after ID challenge with 20 PFU ZIKV over a 12 day period. B) Survival of AG129 after ID challenge with 200 PFU ZIKV over a 12 day period.

FIGS. 6A-B. Sequence of a vector with an exemplary coding sequence to express prM/E (SEQ ID NO:5).

FIG. 7. Schematic of a pCMV (A) and pTriex4-neo (B) vector for expression of prM/E.

FIG. 8A-C. Images showing GFP expression in HEK293 cells. A) pTri px4-neo GFP expression, B) pCMV GFP expression, and C) pCMV GFP expression.

FIG. 9. Western blot analysis of pTriex versus pCMV prM/E expression. Lane 1: Zika virus +; lanes 3,9: pCMV-GFP cells (pt.) and supernatant (sup.); lanes 4,10: pCMV-Columbia pt., sup.; lanes 5,11: pCMV-French-Poly pt., sup.; lanes 6, 12: pTriex-Columbia pt., sup.; and lanes 7, 13: pTriex-French-Poly pt., sup.

FIG. 10. Anti-Zika antibodies in mice before and after VLP exposure. Mice were injected IP with about 10⁶ TCID₅₀ of ZIKV. 5 weeks later the mice were bled, then injected with crude VLP supernatant. Mice were bled 7 days after injection and antibodies analyzed by ZIKV ELISA.

FIG. 11. Western blot of sucrose purified VLPs. Lane 1: marker; lane 2: VLP 100,000 g precipitation; lane 3: Zika virus +; lane 4: pCMV—French-Poly post sucrose purification; and lane 5: pCMV-GFP post sucrose purification. Cells in T-75 flasks were transfected with pCMV-prM/E, or pCMV-GFP, and supernatants were collected after 3 days, then clarified by centrifugation (15,000 g, 30 minutes), then layered onto a 20% sucrose cushion, and pelleted at 112,000 g for 3.5 hours.

FIG. 12. Sucrose fractional analysis. Lane 1: marker; lane 2: Zika virus +; lane 3: Cell debris (pt.) from clarification step; lane 4: Supernatant above sucrose cushion post centrifugation; lane 5: marker; lane 6: VLP post purification batch 1: days 0-3; and lane 7: VLP post purification batch 2: days 3-10. A second batch was harvested from transfected flasks (days 3-10). Purified as before, fractions from each sucrose purification step were analyzed to ensure there was no loss during purification.

FIG. 13. Comparison of protein expression for VLPs produced from pCMV and pTriex constructs.

FIG. 14. Mouse study. 11 AG129 mice of mixed sex and age were used. VLPs were administered IM along with 1 mg Alum. Challenge virus (100 PFU) was administered ID.

FIG. 15. Antibody levels two weeks post boost.

FIG. 16. Survival and morbidity. All controls were moribund on day 9.

FIGS. 17A-C. Dose response of ZIKVLPS in AG129 mice. A-B) PRNT₅₀ and PRNT₉₀ values (+/−SD) of serum samples taken from AG129 mice administered a prime and boost of 0.45 μg (A) or a prime only of 3.0 μg (B) ZIKVLPs pre and post challenge. C) Survival of 11 week old AG129 after ID challenge with 200 PFU ZIKV over a 14 day period.

FIGS. 18A-C. Protection of ZIKVLPS in BALB/c mice. A) PRNT₅₀ and PRNT₉₀ values (+/−SD) of serum samples taken from BALB/c mice administered a prime only of 3.0 μg ZIKVLPs post challenge. B) Viremia (+/−SD) in serum samples from mice two days post challenge by qRT-PCR. Values are total RNA copies per reaction. C) Average weight loss (+/−SD) of BALB/c mice after ID challenge with 200 PFU ZIKV over a 14 day period.

### DETAILED DESCRIPTION

### Definitions

As used herein, the terms “isolated” refers to in vitro preparation, isolation of a nucleic acid molecule such as a vector or plasmid of the invention or a virus-like particle of the invention, so that it is not associated with in vivo substances, or is substantially purified from in vitro substances. An isolated virus-like particle preparation is generally obtained by in vitro culture and propagation and is substantially free from infectious agents. As used herein, “substantially free” means below the level of detection for a particular infectious agent using standard detection methods for that agent. As used herein, the term “recombinant nucleic acid” or “recombinant DNA sequence or segment” refers to a nucleic acid, e.g., to DNA, that has been derived or isolated from a source, that may be subsequently chemically altered in vitro, so that its sequence is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

A signal peptide (sometimes referred to as signal sequence, secretory signal, e.g., an Oikosin 15 secretory signal, targeting signal, localization signal, localization sequence, transit peptide, leader sequence or leader peptide) is a short (about 5 to 30 amino acids long) peptide present at the N-terminus of proteins that are destined towards the secretory pathway. These proteins include those that reside either inside certain organelles (the endoplasmic reticulum, golgi or endosomes), secreted from the cell, or inserted into most cellular membranes. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved. Signal sequences generally have a tripartite structure, consisting of a hydrophobic care region (h-region) flanked by an n- and c-region. The latter contains the signal peptidase (SPase) consensus cleavage site. Usually, signal sequences are cleaved off co-translationally, the resulting cleaved signal sequences are termed signal peptides.

### Exemplary Embodiments

Zika virus infection transmitted by Aedes mosquitoes is now receiving considerable attention due to its associated with microcephaly and Guillain-Barre syndrome. According to the CDC, there have been over 500 cases of travel-related Zika infections in America to date, with no locally-acquired vector-borne cases reported; in contrast, over 700 cases have been reported in US territories, of which nearly all were locally-transmitted.

Computational analysis has identified ZIKV envelope glycoproteins as a good candidate for vaccine development, as these are the most immunogenic (Shawan, 2015). Several approaches are currently being explored to develop a ZIKV vaccine, including inactivated, recombinant live-attenuated viruses, protein subunit vaccines, or DNA vaccines. A VLP vaccine approach against ZIKV may eliminate concerns of live attenuated vaccines and insufficient inactivation of killed vaccines for pregnant women and other populations at high risk of suffering the devastating effects of ZIKV infections.

VLPs are structurally mimic the conformation of native virions but do not generate progeny viruses (VLPs are “non-infectious”) and do not contain any viral genetic material. VLPs are known to be highly immunogenic and elicit higher titer neutralizing antibody responses than subunit vaccines based on individual proteins (Wang et al., 2013). Such VLPs present viral spikes and other surface components that display linear or conformational epitopes in a repetitive array that effectively results in recognition by B-cells (Metz and Pijlman, 2016). This recognition leads to B cell signaling and MHC class II up-regulation that facilitates the generation of high titer specific antibodies. VLPs from viruses, including hepatitis B virus, West Nile virus and Chikungunya virus, elicit high titer neutralizing antibody responses that contribute to protective immunity in preclinical animal models and in humans (Akahata et al., 2010; Spohn et al., 2010; Wang et al., 2012).

As mentioned above, a VLP vaccine approach against ZIKV eliminates concerns of live attenuated vaccines and insufficient inactivation of killed vaccines for pregnant women and other populations at high risk of suffering the devastating effects of ZIKV infections. The generation of ZIKV-VLPs containing the prM and E genes as well as the immunogenicity and efficacy testing in the AG129 mouse model is described herein. A position in the secretory signal was identified that likely allows for higher than normal levels of VLP secretion, due to the absence of an auto (NS2b-3) cleavage signal. Using bioinformatic signal sequence prediction tools, the putative signal sequences of ZIKV starting from positions aa 98-aa 112 were examined, and a site was selected that putatively resulted in the highest secretion score. The prM and E genes from ZIKV (Colombian isolate; GenBank accession no. KU646827) were combined with a secretory signal (positions aa 98-aa 112), were cloned into a mammalian expression vector (pCMV-prM/E). HEK-293 cells were transfected and supernatants were harvested from the cells at approximately 10 days post transfection. Transfected HEK-293 cells secreted VLPs with relatively high yields, likely due to the inclusion of a secretory signal that allows for higher than normal levels of VLP secretion. The cell supernatants contained a fraction of extracellular particles that were purified by ultracentrifugation though a sucrose cushion. These particles reacted with known ZIKV antibodies by Western Blot. Western blot analysis also revealed relatively high yields of VLPs after purification, indicating the potential for scalable production. To test the efficacy of this VLP vaccine, AG129 mice susceptible to ZIKV were vacinated with 2 μg of total protein (about 400-500 ng of VLPs) formulated with 1 mg of adjuvant, and the mice boosted with the same vaccine two weeks later. At two weeks post boost, serum from vaccinated animals was collected and tested for anti-ZIKV neutralizing antibodies. Three weeks post boost mice were challenged with 200 PFU of ZIKV (about 400 LD₅₀s). All control animals (n=6) died by 9 days post challenge, while vaccinated mice survived with no morbidity/illness (as of 11 days post-challenge). Passive transfer of antibodies from vaccinated mice was efficacious in protecting susceptible mice from Zika infections. Thus, the present findings show the protective efficacy of a ZIKV-VLP vaccine and highlight the important role that neutralizing antibodies play in protection against ZIKV infection. Further, passive transfer may be employed as a treatment for immune-compromised patients that cannot receive a vaccine.

In one embodiment, a recombinant nucleic acid vector is provided comprising a heterologous promoter operably linked to a sequence encoding ZIKV, prM/E. In one embodiment, the vector lacks nucleic acid sequences encoding ZIKV NS1, NS2A, NS2B, NS3, NS4A, NS4B or NS5 and optionally lacks nucleic acid sequences encoding functional ZIKV capsid, e.g., a protein that aggregates so as to form a viral capsid having a diameter of about 50 to 60 nm. In one embodiment, the heterologous promoter is expressed in mammalian cells. In one embodiment, the heterologous promoter is a heterologous viral promoter. In one embodiment, only a portion of ZIKV capsid sequences is included, e.g., a C-terminal portion of a ZIKV capsid that is linked to prM/E sequences as in the polyprotein that is expressed by wild-type flavivirus. In one embodiment, the portion of the capsid sequence includes amino acids 98 to 112 of the capsid protein encoded by SEQ ID NO:1 or a protein having at least 80%, 82%, 85%, 87%, 90%, 92%, 95%, 97%, 99% or more amino acid sequence identity thereto. In one embodiment, the prM/E sequences have at least 80% %, 82%, 85%, 87%, 90%, 92%, 95%, 97%, 99% or more amino acid sequence identity to the prM/E sequences encoded by any one of SEQ ID Nos. 1-3 or 5. In one embodiment, the portion of the capsid sequence lacks a NS2B-3 cleavage site. In one embodiment, the prM/E sequences are operably linked to a heterologous secretion signal. In one embodiment, the vector further comprises an intron and/or enhancer sequence, e.g., 5′ to a prM/E coding sequence.

A recombinant host cell comprising the vector is also provided. In one embodiment, the cell is a mammalian cell. In one embodiment, the cell is a human or simian cell. In one embodiment, the genome of the cell is augmented, e.g., stably augmented, with nucleic acid sequences encoding ZIKV NS2B, e.g., the source of NS2B may be heterologous or homologous to the source for prM/E. In one embodiment, the genome of the cell is augmented, e.g., stably augmented, with nucleic acid sequences encoding ZIKV capsid, e.g., the capsid may be heterologous or homologous to prM/E. In one embodiment, the vector is integrated into the genome of the host cell.

Also provided is a method to prepare ZIKV VLPs. The method includes contacting a culture of isolated host cells that do not express ZIKV NS1, NS2A, NS2B, NS3, NS4A, NS4B or NS5 and optionally do not express functional ZIKV capsid, with the recombinant vector and collecting VLPs from supernatant of the culture. Thus, in one embodiment, the isolated host cells do not have ZIKV sequences prior to contact with the vector. In one embodiment, the collected particles have a diameter of about 10 to 100 nm, e.g., 20 to 60 nm, 40 to 70 nm or 40 to 60 nm. In one embodiment, the host cell expresses ZIKV NS2B. In one embodiment, the host cell expresses ZIKV capsid protein and optionally NS2B.

Further provided is a preparation comprising a ZIKV VLPs. The VLP comprises a lipid bilayer comprising ZIKV prM/E but lacks ZIKV NS1, NS2A, NS2B, NS3, NS4A, NS4B or NS5 and optionally lacks functional ZIKV capsid. Such a preparation may be used in a vaccine or immunogenic composition. The vaccine or immunogenic composition may have about 10 to 1000 μg, e.g., 200 to 400 μg or 400 to 800 μg, or about 1 to about 500 mg, e.g., about 20 to 50 mg, about 100 to 300 or about 300 to 400 mg, of VLP. The vaccine or immunogenic composition may further comprise one or more adjuvants. In one embodiment, an adjuvant is included at about 0.01 to about 10 mg, about 1 to about 20 mg, or about 10 mg to about 100 mg.

Further provided is a method to prevent, inhibit or treat ZIKV infection in a mammal. The method includes administering an effective amount of the recombinant vector, a host cell having the vector or the vaccine or immunogenic composition having the VLPs. In one embodiment, the mammal is a female mammal. In one embodiment, the vector, host cell, vaccine or immunogenic composition is administered intradermally, intramuscularly or intravenously to the mammal.

In one embodiment, a method to passively prevent, inhibit or treat ZIKV infection in a mammal is provided. The method includes obtaining serum or plasma having anti-ZIKV antibodies from a mammal exposed to ZIKV and optionally isolating antibodies from the serum or plasma; and administering an effective amount of the serum or plasma, or isolated antibodies, to a different mammal at risk of or having a ZIKV infection. In one embodiment, the mammal is immunocompromised. In one embodiment, the anti-flavivirus antibodies are isolated from the serum before administration. In one embodiment, the mammal is a human.

### Exemplary Adjuvants

Adjuvants are compounds that enhance the specific immune response against co-inoculated antigens. Adjuvants can be used for various purposes: to enhance the immunogenicity of highly purified or recombinant antigens; to reduce the amount of antigen or the number of immunizations needed for protective immunity; to prime the efficacy of vaccines in newborns, the elderly or immuno-compromised persons; or as antigen delivery systems for the uptake of antigens by the mucosa. Ideally, adjuvants should not induce immune responses against themselves and promote an appropriate immune response (i.e., cellular or antibody immunity depending on requirements for protection). Adjuvants can be classified into three groups: active immunostimulants, being substances that increase the immune response to the antigen; carriers being immunogenic proteins that provide T-cell help; and vehicle adjuvants, being oil emulsions or liposomes that serve as a matrix for antigens as well as stimulating the immune response.

Adjuvant groups include but are not limited to mineral salt adjuvants, e.g., alum-based adjuvants and salts of calcium, iron and zirconium; tensoactive adjuvants, e.g, Quil A which is a saponin derived from an aqueous extract from the bark of Quillaja saponaria: Saponins induce a strong adjuvant effect to T-dependent as well as T-independent antigens. Other adjuvant groups are bacteria-derived substances including cell wall peptidoglycan or lipopolysaccharide of Gram-negative bacteria, that enhance immune response against co-administered antigens and which is mediated through activation of Toll-like receptors; lipopolysaccharides (LPS) which are potent B-cell mitogens, but also activate T cells; and trehalose dimycolate (TCM), which simulates both humoral and cellular responses.

Other adjuvants are emulsions, e.g., oil in water or water in oil emulsions such as FIA (Freund's incomplete adjuvant), Montanide, Adjuvant 65, and Lipovant; liposomes, which may enhance both humoral and cellular immunity; polymeric adjuvants such as biocompatible and biodegradable microspheres; cytokines; carbohydrates; inulin-derived adjuvants, e.g., gamma inulin, a carbohydrate derived from plant roots of the Compositae family, is a potent humoral and cellular immune adjuvant and algammulin, which is a combination of γ-inulin and aluminium hydroxide. Other carbohydrate adjuvants include polysaccharides based on glucose and mannose including but not limited to glucans, dextrans, lentinans, glucomannans, galactomannans, levans and xylans.

Some well known parenteral adjuvants, like MDP, monophosphoryl lipid A (MPL) and LPS, also act as mucosal adjuvants. Other mucosal adjuvants poly(DL-lactide-coglycolide) (DL-PLG), cellulose acetate, iminocarbonates, proteinoid microspheres, polyanhydrides, dextrans, as well as particles produced from natural materials like alginates, geletine and plant seeds.

Adjuvants for DNA immunizations include different cytokines, polylactic microspheres, polycarbonates and polystyrene particles.

In one embodiment, adjuvants useful in the vaccines, compositions and methods described herein include, but are not limited to, mineral salts such as aluminum salts, calcium salts, iron salts, and circonium slats, saponin, e.g., Quid A including QS21, squalene (e.g., AS03), TLR ligands, bacterial MDP (N-acetyl muramyl-L-alanyl-D-isoglutamine), lipopolysaccharide (LPS), Lipid A, montanide, Adjuvant 65, Lipovant, Incomplete Freund's adjuvant (IFA), liposmes, microparticles formed of, for example, poly(D,L-lactide (coglycolide)), cytokines, e.g., IFN-gamma or GMCSF, or carbohydrates such as gamma inulin, glucans, dextrans, lentinans, glucomannans and/or glactomannans.

### Pharmaceutical Compositions

Pharmaceutical compositions of the present invention, suitable for inoculation or for parenteral or oral administration, comprise flavivirus VLPs, optionally further comprising sterile aqueous or non-aqueous solutions, suspensions, and emulsions. The compositions can further comprise auxiliary agents or excipients, as known in the art. See, e.g., Berkow et al., 1987; Avery's Drug Treatment, 1987. The composition of the invention is generally presented in the form of individual doses (unit doses).

Vaccines may contain about 0.1 to 500 ng, 0.1 to 500 μg, or 1 to 100 μg, of VLPs. In one embodiment, the vaccine may contain about 100 μg to about 500 μg of VLPs. In one embodiment, the vaccine may contain about at least 100 ng of VLPs. In one embodiment, the vaccine may contain about at least 500 ng of VLPs. In one embodiment, the vaccine may contain about at least 1000 ng of VLPs. In one embodiment, the vaccine may contain about at least 50 μg of VLPs. In one embodiment, the vaccine may contain less than about 750 μg of VLPs. In one embodiment, the vaccine may contain less than about 250 μg of VLPs. In one embodiment, the vaccine may contain less than about 100 μg of VLPs. In one embodiment, the vaccine may contain less than about 40 μg of VLPs. The vaccine forming the main constituent of the vaccine composition of the invention may comprise a combination of different flavirus VLPs, for example, at least two of the three types, Chinese, West African or East African.

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Avery's, 1987.

When a composition of the present invention is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized. Examples of materials suitable for use in vaccine compositions are provided.

A pharmaceutical composition according to the present invention may further or additionally comprise at least one chemotherapeutic compound, for example, immunosuppressants, anti-inflammatory agents or immune enhancers, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

### Pharmaceutical Purposes

The administration of the composition (or the antisera that it elicits) may be for either a “prophylactic” or “therapeutic” purpose. When provided prophylactically, the compositions of the invention which are vaccines, are provided before any symptom of a pathogen infection becomes manifest. The prophylactic administration of the composition serves to prevent or attenuate any subsequent infection or one or more symptoms associated with the disease.

When provided therapeutically, a VLP vaccine is provided upon the detection of a symptom of actual infection. The therapeutic administration of the vaccine serves to attenuate any actual infection. See, e.g., Avery, 1987.

Thus, a VLP vaccine composition of the present invention may thus be provided either before the onset of infection (so as to prevent or attenuate an anticipated infection) or after the initiation of an actual infection.

A composition is said to be “pharmacologically acceptable” if its administration can be tolerated by a recipient patient. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of an infectious flavivirus.

The “protection” provided need not be absolute, i.e., the flavivirus infection need not be totally prevented or eradicated, if there is a statistically significant improvement compared with a control population or set of patients. Protection may be limited to mitigating the severity or rapidity of onset of symptoms of the flavivirus infection.

### Pharmaceutical Administration

A composition of the present invention may confer resistance to one or more pathogens, e.g., one or more flavivirus strains, by either passive immunization or active immunization. In active immunization, an inactivated or attenuated live vaccine composition is administered prophylactically to a host (e.g., a mammal), and the host's immune response to the administration protects against infection and/or disease. For passive immunization, the elicited antisera can be recovered and administered to a recipient suspected of having an infection caused by at least one flavivirus strain.

In one embodiment, the vaccine or immune serum is provided to a mammalian female (at or prior to pregnancy or parturition), under conditions of time and amount sufficient to cause the production of an immune response which serves to protect both the female and the fetus or newborn (via passive incorporation of the antibodies across the placenta or in the mother's milk).

The present invention thus includes methods for preventing or attenuating a disorder or disease, e.g., an infection. As used herein, a vaccine is said to prevent or attenuate an infection if its administration results either in the total or partial attenuation (i.e., suppression) of a symptom or condition of the infection, or in the total or partial immunity of the individual to the disease.

At least one VLP or composition thereof, of the present invention may be administered by any means that achieve the intended purposes, using a pharmaceutical composition as previously described.

For example, administration of such a composition may be by various parenteral routes such as subcutaneous, intravenous, intradermal, intramuscular, intraperitoneal, intranasal, oral or transdermal routes. Parenteral administration can be by bolus injection or by gradual perfusion over time. One mode of using a pharmaceutical composition of the present invention is by intramuscular or subcutaneous application. See, e.g., Avery, 1987.

A typical regimen for preventing, suppressing, or treating a flavivirus related pathology, comprises administration of an effective amount of a vaccine composition as described herein, administered as a single treatment, or repeated as enhancing or booster dosages, over a period up to and including between one week and about 24 months, or any range or value therein.

According to the present invention, an “effective amount” of a composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective doses provided below are not intended to limit the invention and represent suggested dose ranges. However, the dosage will be tailored to the individual subject, as is understood and determinable by one of skill in the art. See, e.g., Avery's, 1987; and Ebadi, 1985.

The invention will be further described by the following non-limiting examples.

### EXAMPLE 1

### Experimental Procedures

### Cells and Viruses

African Green Monkey kidney cells (Vero) and Human embryonic kidney 293 (HEK293) were obtained from ATCC (ATCC; Manassas, Va., USA) and grown in Dulbecco's modified Eagle medium (DMEM) supplemented with 10% fetal bovine serum (FBS; Hyclone, Logan, Utah), 2 mM L-glutamine, 1.5 g/L sodium bicarbonate, 100 U/mL of penicillin, 100 μg/mL of streptomycin, and incubated at 37° C. in 5% CO₂. ZIKV strain H/PF/2013 (GenBank:KJ776791), was obtained from Xavier de Lamballerie (European Virus Archive, Marseille France). Virus stocks were prepared by inoculation onto a confluent monolayer of Vero cells.

### Animals

Mice of the 129/Sv background deficient in alpha/beta interferon (IFN-α/β) and IFN-Υ receptors (AG129 mice) were obtained from B&K Universal Limited (Hull, England) and were bred in the pathogen-free animal facilities of the University of Wisconsin-Madison School of Veterinary Medicine. Groups of mixed sex mice were used for all experiments.

Production and purification of ZIKV VLPs

The prM and E genes of ZIKV strain H/PF/2013 with nascent signal sequence were cloned into a pCM/V expression vector under the control of a cytomegalovirus (CMV) promoter and CMV polyadenylation signal (pCMV-prM/E). Endotoxin free, transfection grade DNA was prepared using Maxiprep kit (Zymo Research, Irvine, Calif.). VLPs were expressed by transfecting 90% confluent monolayers of HEK293 cells in a T-75 flasks with 15 μg of pCMV-prM/E using Fugene HD (Promega, Madison, Wis.) transfection reagent according to manufacturer protocol. The 10 ml supernatant was harvested 72 hours after transfection, and clarified by centrifugation at 15,000 RCF for 30 minutes at 4° C. Clarified supernatants were layered onto a 20% sucrose cushion and ultra-centrifuged in a SW-28 rotor at 112,000 RCF for 3.5 hours at 4° C. Pellet (PT) and supernatant (SUP) fractions at each step were saved for analysis by SDS-PAGE and Western blot. Post sucrose cushion PT were resuspended in Phosphate Buffered Saline (PBS) pH 7.2. Total protein in VLP preparations was quantified by Bradford assay. VLP specific protein was determined by comparing Zika specific bands on SDS-PAGE gels to known concentrations of BSA using ImageJ software.

### Western Blot

VLP fractions were boiled in Laemmli sample buffer (BioRad, Hercules, Calif., USA) and resolved on a 4-20% SDS-PAGE gel (Biorad) by electrophoresis using a Mini-PROTEAN 3 system (BIO-RAD, CA). Gels were electroblotted onto nitrocellulose membranes using a Turboblot® system. Membranes were blocked in 5% (W/V) skim milk and probed with mouse hyper immune ascites fluid primary antibody (1:5000) and goat anti-mouse HRP conjugated secondary antibody (1:5000). Membranes were developed using a solid phase 3,3′,5,5′-tetramethylbenzidine (TMB) substrate system.

### Transmission Electron Microscopy

Samples were negatively stained for electron microscopy using the drop method. A drop of sample was placed on a Pioloform™ (Ted Pella, Inc.) carbon-coated 300 Mesh Cu grid, allowed to adsorb for 30 seconds, and the excess removed with filter paper. Next, a drop of methylamine tungstate or uranyl acetate (Nano-W, Nanoprobes Inc.) was placed on the still wet grid, and the excess removed. The negatively stained sample was allowed to dry, and was documented in a Philips CM120 (Eindhoven, The Netherlands) transmission electron microscope at 80 kV. Images were obtained using a SIS MegaView III digital camera (Soft Imaging Systems, Lakewood, Color.).

### Vaccination and Viral Challenge

For VLP formulations, 0.45 μg of sucrose cushion purified VLPs was mixed with 0.2% Imject Alum (Thermo Scientific) according to manufacturer's protocol. Groups of AG129 mice were injected intramuscularly (IM) with VLPs mixed with alum (n=5) or PBS mixed with alum (n=6) at 6 weeks of age, and again at 8 weeks of age. Sub-mandibular blood draws were performed pre boost and pre challenge to collect serum for analysis by neutralization assays and for passive transfer studies.

Vaccinated mice were challenged with 200 PFU of ZIKV strain H/PF/2013 in 25 μl volumes by intradermal (ID) injection into the right hind footpad. Following infection, mice were monitored daily for the duration of the study. Mice that were moribund or that lost greater than 20% of starting weight were humanely euthanized. Sub-mandibular blood draws were performed on day two post challenge (PC) and serum collected to measure viremia.

For passive transfer studies, 5 naive mice were injected intraperitoneally (IP) with 500 μl of pooled serum from VLP vaccinated, diluted serum (1:5 n=4, 1:10, n=4), or serum from PBS/alum (n=5) treated mice. At 12 hours post transfer, mice were challenged with 20 PFU in 25 μl as above.

### Viremia Assays

Viremia was determined by TCID50 assay. Briefly, serum was serially diluted ten-fold in microtiter plates 263 and added to duplicate wells of Vero cells in 96-well plates, incubated at 37° C. for 5 days, then fixed and 264 stained with 10% (W/V) crystal violet in 10% (V/V) formalin. Plates were observed under a light microscope to determine the 50% tissue culture infective doses (TCID50s). Serum samples were also tested for viral RNA copies by qRT-PCR. RNA was extracted from 0.02 ml of serum using the ZR Viral 267 RNA Kit (Zymo Research, Irvine, Calif.). Viral RNA was quantified by qRT-PCR using the primers and probe designed by Lanciotti et al. (Lanciotti et al., 2008). The qRT-PCR was performed using the iTaq Universal Probes One-Step Kit (BioRad, Hercules, Calif.) on an iCycler instrument (BioRad, Hercules, Calif.). Primers and probe were used at final concentrations of 500 nM and 250 nM respectively. Cycling conditions were as follows: 50° C. for 10 minutes and 95° C. for 2 minutes, followed by 40 cycles of 95° C. for 15 seconds and 60° C. for 30 seconds. Virus concentration was determined by interpolation onto an internal standard curve made up of a 5-point dilution series of in vitro transcribed RNA.

### Neutralization Assay

Serum antibody titers were deteiliiined by microneutralization assay. Briefly, serum was incubated at 56° C. for 30 minutes to inactivate complement and then serially diluted two-fold in microtiter plates. 200 PFUs of virus were added to each well and incubated at 37° C. for 1 hour. The virus-serum mixture was added to duplicate wells of Vero cells in 96-well plates, incubated at 37° C. for 5 days, then fixed and stained with 10% (W/V) crystal violet in 10% (V/V) formalin, then observed under a light microscope. The titer was determined as the serum dilution resulting in the complete neutralization of the virus.

### Plaque Reduction Neutralization Test

Serum samples were serially diluted, mixed with 200 PFU of the ZIKV H/PF/2013 strain and incubated for 1 hour at 37° C. This serum/virus mixture was added to confluent layers of Vero cells in 96 well plates and incubated for 1 hour at 37° C., after which the serum/virus mixture was removed and overlay solution (3% CMC, 1×DMEM, 2% FBS and 1×Anti/Anti) was added. After 48 hours of infection, the monolayers were fixed with 4% PFA, washed twice with PBS, and then incubated with ZIKV hyperimmune mouse ascitic fluid (1:2000, UTMB) diluted in blocking solution (1×PBS, 0.01% Tween-20 and 5% Milk) and incubated overnight at 4° C. Plates were washed three times with PBS-T and then peroxidase-labeled goat anti-mouse secondary antibody (1:2000) was incubated on monolayers for 2 hours at 37° C. Following incubation, cells were washed a final three times with PBS-T and developed using 3-amino-9-ethylcarbazole (AEC)-peroxidase substrate. The amount of formed foci were counted using an 292 ELISPOT plate reader (ImmunoSPOT-Cellular Technology); quality control was performed to each scanned well to ensure accurate counting. Neutralization percentages (Nx) were calculated per sample/replicate/dilution as follows:

$$Nx = \{ 100 - [ 100 ( \frac{A}{Control} )$$

Where A corresponds to the amount of foci counted in the sample and Control is the geometric mean of foci counted from wells treated with cells and virus only. Data of corresponding transformed dilutions (Log(1/Dilution)) against neutralization percentages per sample was plotted and fitted to a sigmoidal dose-299 response curve to interpolate PRNT₅₀ and PRNT₉₀ values (GraphPad Prism software).

SEQ ID NO: 9:
mknpkkksgg frivnmlkrg varvspfggl krlpaglllg hgpirmvlai laflrftaik
pslglinrwg svgkkeamei ikkfkkdlaa mlriinarke kkrrgadtsv givgllltta
maaevtrrgs ayymyldrnd ageaisfptt lgmnkcyiqi mdlghmcdat msyecpmlde
gvepddvdcw cnttstwvvy gtchhkkgea rrsrravtlp shstrklqtr sqtwlesrey
tkhlirvenw ifrnpgfala aaaiawllgs stsqkviylv milliapays ircigvsnrd
fvegmsggtw vdvvlehggc vtvmaqdkpt vdielvtttv snmaevrsyc yeasisdmas
dsrcptqgea yldkqsdtqy vckrtlvdrg wgngcglfgk gslvtcakfa cskkmtgksi
qpenleyrim lsvhgsqhsg mivndtghet denrakveit pnspraeatl ggfgslgldc
eprtgldfsd lyyltmnnkh wlvhkewfhd iplpwhagad tgtphwnnke alvefkdaha
krqtvvvlgs qegavhtala galeaemdga kgrlssghlk crlkmdklrl kgvsyslcta
aftftkipae tlhgtvtvev qyagtdgpck vpaqmavdmq tltpvgrlit anpviteste
nskmmleldp pfgdsyivig vgekkithhw hrsgstigka featvrgakr mavlgdtawd
fgsvggalns lgkgihqifg aafkslfggm swfsqiligt llmwlglntk ngsislmcla
lggvliflst avsadvgcsv dfskketrcg tgvfvyndve awrdrykyhp dsprrlaaav
kqawedgicg issvsrmeni mwrsvegeln aileengvql tvvvgsvknp mwrgpqrlpv
pvnelphgwk awgksyfvra aktnnsfvvd gdtlkecplk hrawnsflve dhgfgvfhts
vwlkvredys lecdpavigt avkgkeavhs dlgywiesek ndtwrlkrah liemktcewp
kshtlwtdgi eesdliipks lagplshhnt regyrtqmkg pwhseeleir feecpgtkvh
veetcgtrgp slrsttasgr vieewccrec tmpplsfrak dgcwygmeir prkepesnlv
rsmvtagstd hmdhfslgvl villmvgegl kkrmttkiii stsmavlvam ilggfsmsdl
aklailmgat faemntggdv ahlaliaafk vrpallvsfi franwtpres mllalascll
qtaisalegd lmvlingfal awlairamvv prtdnitlai laaltplarg tllvawragl
atcggfmlls lkgkgsvkkn lpfvmalglt avrlvdpinv vglllltrsg krswppsevl
tavglicala ggfakadiem agpmaavgll ivsyvvsgks vdmyieragd itwekdaevt
gnsprldval desgdfslve ddgppmreii lkvvlmticg mnpiaipfaa gawyvyvktg
krsgalwdvp apkevkkget tdgvyrvmtr rllgstqvgv gvmqegvfht mwhvtkgsal
rsgegrldpy wgdvkqdlvs ycgpwkldaa wdghsevqll avppgerarn iqtlpgifkt
kdgdigaval dypagtsgsp ildkcgrvig lygngvvikn gsyvsaitqg rreeetpvec
fepsmlkkkq ltvldlhpga gktrrvlpei vreaiktrlr tvilaptrvv aaemeealrg
lpvrymttav nvthsgteiv dlmchatfts rllqpirvpn ynlyimdeah ftdpssiaar
gyistrvemg eaaaifmtat ppgtrdafpd snspimdtev evperawssg fdwvtdhsgk
tvwfvpsvrn gneiaacltk agkrviqlsr ktfetefqkt khqewdfvvt tdisemganf
kadrvidsrr clkpvildge rvilagpmpv thasaaqrrg rigrnpnkpg deylygggca
etdedhahwl earmlldniy lqdgliasly rpeadkvaai egefklrteq rktfvelmkr
gdlpvwlayq vasagitytd rrwcfdgttn ntimedsvpa evwtrhgekr vlkprwmdar
vcsdhaalks fkefaagkrg aafgvmealg tlpghmterf qeaidnlavl mraetasrpy
kaaaaqlpet letimllgll gtvslgiffv lmrnkgigkm gfgmvtlgas awlmwlseie
pariacvliv vflllvvlip epekqrspqd nqmaiiimva vgllglitan elgwlertks
dlshlmgrre egatigfsmd idlrpasawa iyaalttfit pavqhavtts ynnyslmama
tqagvlfgmg kgmpfyawdf gvpllmigcy sqltpltliv aiillvahvm ylipglqaaa
araaqkrtaa gimknpvvdg ivvtdidtmt idpqvekkmg qvlliavavs sailsrtawg
wgeaqalita atstlwegsp nkywnsstat slcnifrgsy lagasliytv trnaglvkrr
gggtgetlge kwkarlnqms alefysykks gitevcreea rralkdgvat gghavsrgsa
klrwlvergy lqpygkvidl gcgrggwsyy aatirkvqev kgytkggpgh eepmlvqsyg
wnivrlksgv dvfhmaaepc dtllcdiges ssspeveear tlrvlsmvgd wlekrpgafc
ikvlcpytst mmetlerlqr rvggglvrvp lsrnsthemy wvsgaksnti ksvsttsqll
lgrmdgprrp vkyeedvnlg sgtravvsca eapnmkiign rierirseha etwffdenhp
yrtwayhgsy eaptqgsass lingvvrlls kpwdvvtgvt giamtdttpy gqqrvfkekv
dtrvpdpqeg trqvmsmvss wlwkelgkhk rprvctkeef inkvrsnaal gaifeeekew
ktaveavndp rfwalvdker ehhlrgecqs cvynmmgkre kkqgefgkak gsraiwymwl
garflefeal gflnedhwmg rensgggveg lglqrlgyvl eemsripggr myaddtagwd
trisrfdlen ealitnqmek ghralalaii kytyqnkvvk vlrpaekgkt vmdiisrqdq
rgsgqvvtya lntftnlvvq lirnmeaeev lemqdlwllr rsekvtnwlq sngwdrlkrm
avsgddcvvk piddrfahal rflndmgkvr kdtqewkpst gwdnweevpf cshhfnklhl
kdgrsivvpc rhqdeligra rvspgagwsi retaclaksy aqmwqllyfh rrdlrlmana
icssvpvdwv ptgrttwsih gkgewmtted mlvvwnrvwi eendhmedkt pvtkwtdipy
lgkredlwcg slighrprtt waenikntvn mvrriigdee kymdylstqv rylgeegstp
gvl

### RESULTS

### Expression and Purification of Soluble, Zika VLPs

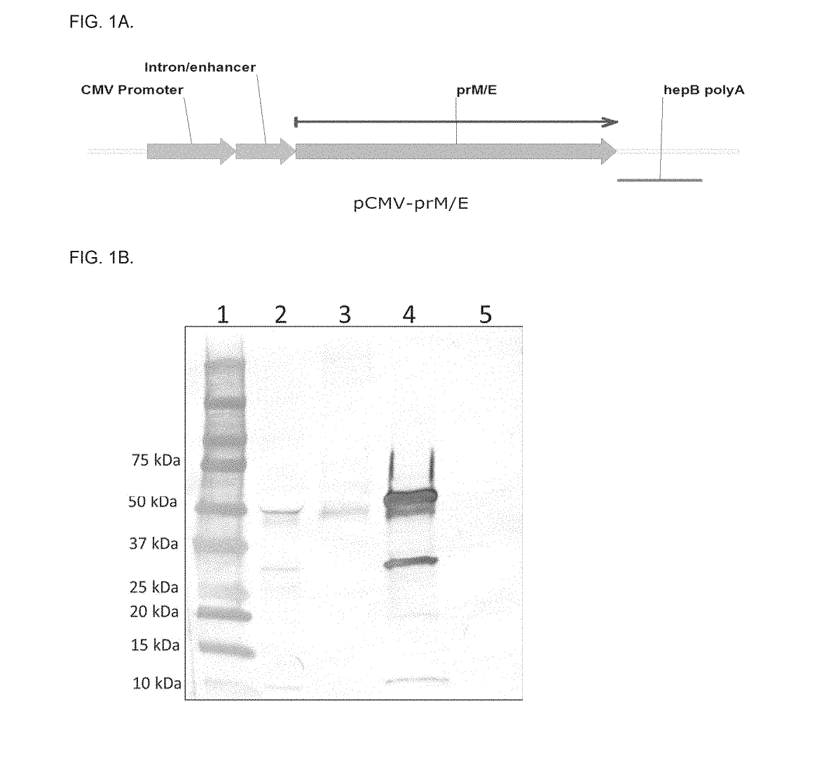

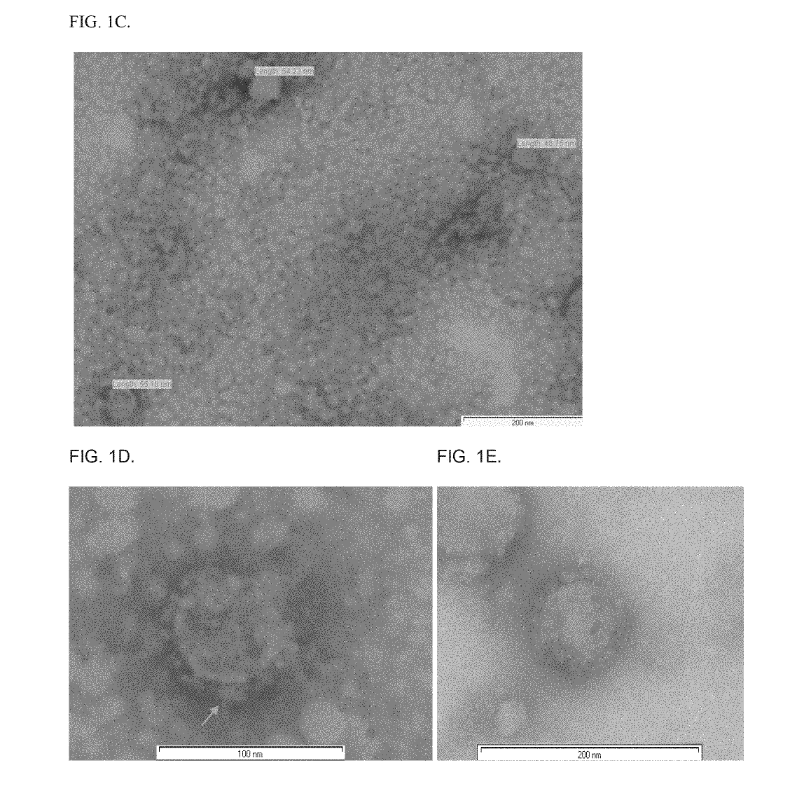

To generate Zika VLPs (ZIKVLPs), the prM/E genes with a native signal sequence were cloned into a pCMV expression vector (pCMV-prM/E) (FIG. 1A), transfected HEK293 cells and harvested supernatants (supe) 3 days post transfection. 78 μg total protein was recovered from post sucrose purification of which 21.6 μg was VLP protein. Western blot analysis of this pCMV-prM/E supe. revealed expression of about 50 kDa size band (FIG. 1B, lane 2) that corresponded in size to the predicted size of the Zika viurs E gene, and additionally matched positive control Zika virus stocks (FIG. 1B, lane 3). To test the hypothesis that expression of Zika prM and E genes spontaneously form extracellular particles, supernatants from pCMV-prM/E and pCMV-GFP (negative control) transfected cells were centrifuged on a sucrose cushion (SC) sufficient for pelleting of flavi virus particles from cell culture proteins (Merino-Ramos et al., 2014). pCMV-prM/E SC purified pellet (pt) appeared to contain high levels of E protein, while pCMV-GFP pt. did not, indicating that staining was specific to expression of 100 prM and E genes.

To determine if the immune reactive extracellular particles were virus like in nature, transmission electron microscopy (TEM) was performed on pCMV-prM/E SC pt. material. TEM revealed flavi virus 103 like particles with a size that ranged from 30-60 nm (data not show), and a typical size of about 50 nm (FIG. 1C). High magnification images demonstrated surface structures characteristic of flaviral envelope proteins (FIGS. 1D, E).

### Administration of ZIKVLPs is Immunogenic and Protects Highly ZIKV Susceptible α/β/γ Interferon Deficient Mice

Mice that received ZIKVLPs developed low levels (GMT=1:9.2) of neutralizing antibodies (nAbs) at 109 two weeks post administration, that increased two weeks after boost (GMT=1:32). Five weeks after primary vaccination, all mice were challenged with 200 PFU of ZIKV by the ID route. Mice administered ZIKVLP maintained weight, while mice that received PBS/alum experienced significant weight loss associated morbidity throughout the challenge period.

All control mice (n=6) died 9 days after ZIKV challenge. Mice administered ZIKVLP survived with no apparent morbidity. Finally, ZIKVLP vaccinated mice had significantly lower levels of viremia on day 2 post challenge than control mice detected by qRT-PCR (p=0.0356) and 116 TCID50 assay (p=0.0493).

### ZIKVLPs Elicit Plaque Reducing Neutralizing Antibody Titers in Mice That Can Be Passively Transferred to Naïve Mice.

The plaque reduction neutralization test (PRNT) assay is widely considered to be the “gold standard” for characterizing and quantifying circulating levels of anti-dengue and other flaviviral neutralizing antibodies (nAb) (Thomas et al., 2009). A PRNT assay was developed for rapidly measuring ZIKV specific neutralizing antibodies. Pooled serum samples collected from mice pre-challenge, as well as individual serum samples collected from mice post-challenge were tested by this PRNT assay. Pre-challenge, pooled serum from mice administered ZIKVLP had a calculated 90% plaque reduction (PRNT₉₀) titer of 1:34. The PRNT₉₀ titer increased 2 weeks post challenge (GMT=126 662).

To test the role of anti-ZIKV antibodies in protection against challenge, groups of mice received ZIKVLP 128 antiserum, undiluted (n=5), diluted 1:5 (n=4), or 1:10 (n=4). As a negative control mice (n=5) were transferred serum from mice previously vaccinated with PBS alum.

Negative control mice rapidly lost weight starting after day 7 and all died day 9 post challenge. Mice that received undiluted serum maintained weight throughout the 12 day period post challenge, and showed no signs of infection. Mice that received diluted anti-ZIKV antibodies were not protected from challenge, although survival and weigh loss were slightly extended relative to negative control mice 134.

### DISCUSSION

Most experts and public health workers agree that a Zika vaccine is urgently needed. In February 2016, the World Health Organization declared that the recent clusters of microcephaly and other neurological disorders in Brazil constitute a public health emergency of international concern. Their recommendations included enhanced surveillance and research, as well as aggressive measures to reduce infection with Zika virus, particularly amongst pregnant women and women of childbearing age. ZIKV is now receiving considerable attention due to its rapid spread in the Americas, and its association with microcephaly (Mlakar et al., 2016) and Guillain-Barre syndrome (Pinto Junior et al., 2015). In our studies, we designed a ZIKV-virus-like particle (VLP) vaccine, demonstrated expression in vitro by western blot and transmission electron microscopy, and tested the protective efficacy and role of antibodies in protection in the AG129 mouse model.

Although the transfection and purification procedures for this ZIKV-VLP have yet to be optimized, we had an overall calculated yield of 2.2 mg/ml. Similar expression levels have been reported for other flavivirus VLP expression strategies (Pijlman, 2015). Future work will optimize VLP production and purification parameters, which should significantly increase both yield and purity. Stably transfected HEK cells that continuously express VLPs allow for scalable production to meet global demand for a ZIKV vaccine.

ZIKV-VLPs, formulated with alum, induced detectable neutralizing antibodies and protected animals against lethal challenge (>400 LD50s) with no morbidity or weight loss. Pre-challenge GMT neutralizing titers were 1:32, and pooled pre-challenge serum PRNT₉₀ and PRNT₅₀ titers were 1:34 and 1:157 respectively. At a relatively low dose of 450 ng, the present results indicate that the ZIKV VLPs are highly immunogenic. Additionally, the antibody titers we obtained are consistent with those reported for other highly immunogenic flavivirus VLP vaccines (Ohtaki et al., 2010; Pijlman, 2015).

Vaccinated mice challenged with >400 LD50s had low levels of viremia (mean=127, geometric mean=25.4 TCID50/ml) detected after challenge. Copies of RNA ZIKV genomes in serum of mice were significantly higher than levels of viremia. However, the disparity between viral genome copies and viremia has been observed for other flaviviruses including dengue (Bae et al., 2003). Since AG129 mice are highly susceptible to viral challenge, it is possible that the challenge dose given for the active vaccination study was artificially high. Additionally, methods for challenging mice from infected mosquito bite should be developed to most accurately mimic natural infection. Animal studies can determine if the ZIK VLP vaccine can protect female mice from contracting ZIKV during pregnancy using established models for such studies (Miner et al., 2016). ZIK-VLP vaccines may be tested in a non-human primate translational model which most accurately mimics human infection.

A VLP vaccine approach against ZIKV has significant advantages over other technologies as it will eliminate concerns of live attenuated vaccines and insufficient inactivation of killed vaccines for pregnant women and other populations at high risk of suffering the devastating effects of ZIKV infections. In recent years, recombinant virus-like particle (VLP)-based vaccine strategies have been frequently used for novel vaccine design. VLPs are known to be highly immunogenic and elicit higher titer neutralizing antibody responses than subunit vaccines based on individual proteins (Ariano et al., 2010).

The role of neutralizing antibodies in protecting against ZIKV was demonstrated by antibody passive transfer studies as naive AG129 mice receiving pooled serum from VLP vaccinated animals were fully protected. These results are consistent with previous findings that indicate the important role of antibodies in protecting against many mosquito-borne viruses, such as Japanese encephalitis, yellow fever and chikungunya. In this study, full protection was observed when animals received undiluted serum, with no weight loss or other clinical signs observed. While these studies highlight the importance of serum antibodies in ZIKV protection, upcoming studies will determine the minimum antibody titer needed for protection, whether the ZIKV-VLP can elicit CD8+ responses, and the overall role of cellular immunity in protection. It is also important to determine whether anti-ZIKV antibodies elicited by the VLPs play any role in dengue protection or disease enhancement.

In this study, the AG129 IFN receptor-deficient mouse model was used for evaluation of the ZIKV-VLP. Recently, the suitability of mice deficient in IFN-α/β and -γ receptors as an animal model for ZIKV was demonstrated, as they are highly susceptible to ZIKV infection and disease, developing rapid viremic dissemination in visceral organs and brain and dying 7-8 days post-infection (Aliota et al., 2016). The AG129 mouse model exhibits an intact adaptive immune system, despite the lack of an IFN response, and it has been used extensively to evaluate vaccines and antivirals for DENV (Brewoo et al., 2012; Fuchs et al., 2014; Johnson and Roehrig, 1999; Sarathy et al., 2015).

In the present study, aluminum hydroxide (commonly known as alum) was used as the adjuvant for the ZIKV-VLP preparations. Since its first use in 1932, vaccines containing aluminum-based adjuvants have been successfully administered in humans demonstrating excellent safety. A variety of adjuvant formulations may, however, be employed with ZIKV VLPs to enhance immunogenic potential including adjuvants that facilitate antigen dose sparing, enhanced immunogenicity, and/or broadened pathogen protection.

Thus, a VLP based Zika vaccine is described herein that elicits protective antibodies in mice, and is safe, suitable for scalable production, and highly immunogenic. Fast-tracking development of this ZIKV vaccine is a public health priority and is crucial for restoring confidence and security to people who wish to have children or reside in, or visit areas in which ZIKV is endemic.

### EXAMPLE 2

### Exemplary Zika Virus Polyprotein Sequences:

Accession No. KU646827 (Which is Incorporated by Reference Herein)

(SEQ ID NO: 6)
IRCIGVSNRDFVEGMSGGTWVDVVLEHGGCVTVIVIAQDKPTVDIE
LVTTTVSNMAEVRSYCYEASISDMASDSRCPTQGEAYLDKQSDTQYVCKRTLVDRGWG
NGCGLFGKGSLVTCAKFACSKKIVITGKSIQPENLEYRIMLSVHGSQHSGMIVNDTGH
ETDENRAKIVEITPNSPRAEATLGGFGSLGLDCEPRTGLDFSDLYYLMINNKHWLVHK
EWTHDIPLPWELNGADTGTPHWNNKEALVEFKDAHAKRQTVVVLGSQEGAVHTALAGA
LEAEMDGAKGRLSSGHLKCRLKMDKLRLKGVSYSLCTAAFTFTKIPAETLHGTVIVEV
QYAGTDGPCKVPAQIVIAVDMQTLTPVGRLITANPVITESTENSKMMLELDPPFGDSY
IVIGVGEKKITHHAVHRSGSTIGKAFEATVRGAKRMAVLGDTAWDFGSVGGALNSLGK
GIHQIFGAAFKSLFGGMSWFSQILIGTLLMWLGLNTKNGSISLMCLALGGVLIFLSTA
VSADVGCSVDFSKKETRCGTGVFVYNDVEAIATRDRYKYHPDSPRRLAAAVKQAWEDG
ICGISSVSRMENIMWRSVEGELNAILEENGVQLTVVVGSVKNPMWRGPQRLPVPVNEL
PHGWKAWGKSYFVRAAKTNNSFVVDGDTLKECPLKHRAWNSFLVEDHGFGVFHTSVWL
KVREDYSLECDPANTIGTAVKGKEAVHSDLGYWIESEKNDTWRLKRAHLIEMKTCEWP
KSHTLWTDGIEESDLIIPKSLAGPLSHHNTREGYRTQMKGPWHSEELEIRFEECPGTK
ATHVEETCGTRGPSLRSTTASGRVIEEWCCRECTMPPLSFWAKDGCWYGMEIRPRKEP
ESNLVRSMVTAGSTDHMDHFSL
(SEQ ID NO: 1)
atcaggtgca taggagtcag caatagggac tttgtggaag gtatgtcagg tgggacttgg
gttaatgtcg tcttggaaca tggagattgt gtcaccgtaa tggcacaaga caaaccgact
gtcgacatag agctggttac aacaacagtc agcaacatgg cggaggtaag atcctactgc
tatgaggcat caatatcaga catggcttcg gacagccgct gcccaacaca aggtgaagcc
taccttgaca agcaatcaga cactcaatat gtctgcaaaa gaacgttagt ggacagaggc
tggggaaatg gatgtggact ttttggcaaa gggagcctgg tgacatgcgc taagtttgca
tgctccaaga aaatgaccgg gaagagcatc cagccagaga atctggagta ccggataatg
ttgtcagttc atggctccca gcacagtggg atgatcgtta atgacacagg acatgaaact
gatgagaata gagcgaaggt tgagataacg cccaattcac caagagccga agccaccctg
gggagttttg aaagcctaag acttgattgt gaaccgagga caggccttaa cttttcagat
ttgtattact tgactatgaa taacaagcac tggttggttc acaaggagtg gttccacgac
attccattac cttggcacgc tggggcagac accggaactc cacactggaa caacaaagaa
gcactggtag agttcaagga cgcacatgcc aaaaggcaaa ctgtcgtggt tctagggagt
caggaagaag cagttcacac gacccttgct ggagctctgg aggctgagat gaatggtgca
aagggaaggc tgtcctctgg ccacttgaaa tgtcgcctga aaatggacaa acttagattg
aagggcgtgt catactcctt gtgtaccgca gcgttcacat tcaccaagat cccggctgaa
acactgcacg ggacagtcac agtggaggta cagtacgcag ggacagatgg accttgcaag
gttccagctc agatggcgat ggacatgcaa actctgaccc cagttgggag gttgataacc
gctaaccccg taatcactga aagcactgag aactctaaga tgatgctgga acttgatcca
ccatttgggg actcttacat tgtcatagga gtcggggaga agaagatcac ccaccactgg
cacaggagtg gcagcaccat tggaaaagca tttgaagcca ctgtgagagg tgccaagaga
atggcagtct tgggagacac aacctgggac tttggatcag ttggaggcgc tctcaactca
ttgggcaagg gcatccatca aatttttgga gcagctttca aatcattgtt tggaggaatg
tcctggttct cacaaattct cattggaacg ttgctgatgt gattggatct gaacacaaag
aatggatcta tttcccttat gtgcttggcc ttagggggag tgttgatctt cttatccaca
gccatctctg ctgatgtgag gtgctcggtg gacttctcaa agaaggagac gagatatggt
acaggggtgt tcgtctataa cgacgttgaa gcctggaggg acaggtacaa gtaccatcct
gactcccccc gtagattggc agcagcagtc aagcaagcct gggaagatgg tatctgcggg
atctcctctg tttcaagaat ggaaaacatc atgtggagat cagtagaagg ggagctcaac
gcaatcctgg aagagaatgg agttcaactg acggtcgttg tgggatctgt aaaaaacccc
atgtggagag gtccacagag attgcccgtg cctgtgaacg agctgcccca cggctggaag
gcttagggga aatcgtactt cgtcaaagca gcaaagacaa ataacagctt tgtcgtggat
ggtgacacac tgaaggaatg cccactcaaa catagagcat ggaacagctt tcttgtggag
gatcatgggt tcgaggtatt tcacactagt gtctggctca aggttagaga agattattca
ttagagtatg atccagccgt tattggaaca gctgttaagg gaaaagaggc tatacacagt
gatctagact actgaattga gagtgagaag aatgacacat ggagactgaa gagggcccat
ctgatcgaga tgaaaacatg tgaatggcca aagtcccaca cattgtggac agatggaata
gaagagagtg atctgatcat acccaagtct ttagctgggc cactcagcca tcacaatacc
agagagggct acaggaccca aatgaaaggg ccatggcaca gtgaagagct tgaaattcgg
tttaaggaat acccaggcac taaggtccac gtgaaggaaa catgtggaac aagagaacca
tctctgagat caaccactgc aagcggaagg gtgatcgagg aatggtgctg cagggagtgc
acaatgcccc cactgtcgtt ctgggctaaa gatggctgtt ggtatggaat ggagataagg
cccaggaaag aaccagaaag caacttagta aggtcaatgg tgactgcagg atcaactgat
cacatagatc acttctccct t
KU955593 (full-length)
(SEQ ID NO: 7)
MKKPKKKSGGFRIVNMLKRGVARVSPFGGLKRLPAGLLLGHGPI
RMVLAILAFLRFTAIKPSLGLINRWGSVGKKEAMEIIKKFKKDLAAMLRIINARKEKK
RRGTDTSVGIVGLLLTTAMAVEVTRRGNAYYMYLDRSDAGEAISFPTTMGMNKCYIQI
MDLGHMCDATMSYECPMLDEGVEPDDVDCWCNTTSTWVVYGTCHHKKGEARRSRRAVT
LFSKSTRKLQTRSQTWLESREYTKHLIRVENWIFRNPGFALAAAAIAWLLGSSTSQKV
IYLVMILLIAPAYSIRCIGVSNRDFVEGMSGGTWVDVVLEHGGCVTVMAQDKPTVDIE
LVTTTVSNMAEVRSYCYEASISDMASDSRCFTQGEAYLDKQSDTQYVCKRTLVDRGWG
NGCGLFGKGSLVTCAKFACSKKMTGKSIQPENLEYRIMLSVHGSQHSGMIVNDTGHET
DENRAKVEITPNSPRAEATLGGFGSLGLTCEPRTGLDFSDLYYLTMNNKHWLVHKEWF
HDIPLPWHAGADTGTPHWNNKEALVEFKDAHAKRQTVVVLGSQEGAVHTALAGALEAE
MDGAKGRLSSGHLKCRLKMDKLRLKGVSYSLCTAAFTFTKIPAETLHGTVTVEVQYAG
TDGPCKVPAQMAVDMQTLTPVGRLITANPVITESTENSKMMLELDPPFGDSYIVIGVG
EKKITHHWHRSGSTIGKAFEATVRGAKRMAVLGDTAWDFGSVGGALNSLGKGIHQIFG
AAFKSLFGGMSWFSQILIGTLLVWLGLNTKNGSISLMCLALGGVLIFLSTAVSADVGC
SVDFSKKETRCGTGVFVYNDVEAWRDRYKYHPDSPRRLAAAVKQAWEDGICGISSVSR
MENIMWRSVEGELNAILEENGVQLTVVVGSVKNPMWRGPQRLPVPVNELPHGWKAWGK
SYFVRAAKTNNSFVVDGDTLKECPLKHRAWHSFLVEDHGFGVFHTSVWLKVREDYSLE
CDPAVIGTAAKGKEAVHSDLGYWIESEKNDTWRLKRAHLIEMKTCEWPKSHTLWTDGI
EESDLIIPKSLAGPLSHHNTREGYRTQMKGPWHSEELEIRFEECPGTKVHVEETCGTR
GPSLRSTTASGRVIEEWCCRECTMPPLSFRAKDGCWYGMEIRPRKEPESNLVRSMVTA
GSTDHMDKFSLGVLVILLMVQEGLKKRMTTKIIISTSMAVLVAMILGGFSMSDLAKLA
ILMGATFAEMNTGGDVAHLALIAAFKVRPALLVSFIFRANWTPRESMLLALASCLLQT
AISALEGDLMVPINGFAIAWLAIRAMVVPRTDNITLAILAALTPLARGTLLVAWRAGL
ATCGGFMLLSLKGKGSVKKNLPFVMALGLTAVRLVDPINVVGLLLLTRSGKRSWPPSE
VLTAVGLICALAGGFAKADIEMAGPMAAVGLLIVSYVVSGKSVDMYIERAGDITWEKD
AEVTGNSPRLDVALDESGDFSLVEDDGPPMREIILKVVLMAICGMNPIAIFFAAGAWY
VYVKTGKRSGALWDVPAPKEVKKGETTDGVYRVMTRRLLGSTQVGVGVMQEGVFHTMW
HVTKGSALRSGEGRLDPYWGDVKQDLVSYCGPWKLDAAWDGHSEVQLLAVPPGERARN
IQTLPGIFKTKDGDIGAVALDYPAGTSGSFILDKCGRVIGLYGNGVVIKNGSYVSAIT
QGRREEETPVECFEPSMLKKKQLTVLDLHPGAGKTRRVLPEIVREATKTRLRTVTLAP
TRVVAAEMEEALRGLPVRYMTTAVNVTKSGTEIVDLMCHATFTSRLLQPIRVPNYNLY
IMDEAHFTDPSSIAARGYISTRVEMGEAAAIFMTATPPGTRDAFPDSNSPIMDTEVEV
PERAWSSGFDWVTDHSGKTVWFVPSVRNGNEIAACLTKAGKRVIQLSRKTFETEFQKT
KHQEWDFVVTTDISEMGANFKADRVIDSRRCLKPVILDGERVILAGPMPVTHASAAQR
RGRIGRNPNKPGDEYLYGGGCAETDEDHAHWLEARMLLDNIYLQDGLIASLYRPEADK
VAAIEGEFKLRTEQRKTFVELMKRGDLPVWLAYQVASAGITYTDRRWCFDGTTNNTIM
EDSVPAEVWTRYGEKRVLKPRWMDARVCSDHAALKSFKEFAAGKRGAAFGVMEALGTL
PGHMTERFQEAIDNLAVLMRAETGSRPYKAAAAQLPETLETIMLLGLLGTVSLGIFFV
LMRNKGIGKMGFGMVTLGASAWLMWLSEIEPARIACVLIVVFLLLVVLIPEPEKQRSP
QDNQMAIIIMVAVGLLGLITANELGWLERTKSDLSHLMGRREEGATIGFSMDIDLRPA
SAWAIYAALTTFITPAVQHAVTTSYNNYSLMAMATQAGVLFGMGKGMPFYAWDFGVPL
LMIGCYSQLTPLTLIVAIILLVAHYMYLIPGLQAAAARAAQKRTAAGIMKNPVVDGIV
VTDIDTMTIDPQVEKKMGQVLLIAVAVSSAILSRTAWGWGEAGALITAATSTLWEGSP
NKYWNSSTATSLCNIFRGSYLAGASLIYTVTRNAGLVKRRGGGTGETLGSKWKARLNQ
MSALEFYSYKKSGITEVCREEARRALKDGVATGGHAVSRGSAKLRWLVERGYLQPYGK
VIDLGCGRGGWSYYAATIRKVQEVKGYTKGGPGHEEPMLVQSYGWNIVRLKSGVDVFH
MAAEPCDTLLCDIGESSSSPEVEEARTLRVLSMVGDWLEKRPGAFCIKVLCPYTSTMM
ETLERLQRRYGGGLVRVPLSRNSTHEMYWVSGAKSNTIKSVSTTSQLLLGRMDGPRRP
VKYEEDVNLGSGTRAVVSCAEAPNMKIIGNRIERIRSEHAETWFFDENKPYRTWAYKG
SYEAPTQGSASSLINGVVRLLSKPWDVVTGVTGIAMTDTTPYGQQRVFKEKVDTRVPD
PQEGTRQVMSMVSSWLWKELGKHKRFRVCTKEEFINKVRSNAALGAIFEEEKEWKTAV
EAVNDPREWALVDKEREHHLRGECQSCVINMMGKREKKQGEFGKAKGSRAIWYMWLGA
RFLEFEALGFLNEDHWMGRENSGGGVEGLGLQRLGYVLEEMSRIPGGRMYADDTAGWD
TRISRFDLENEALTINQMEKGHRALALAIIKYTYQNKVVKVLRPAEKGKTVMDITSRQ
DQRGSGQVVTYALNTFTNLVVQLTRNMEAEEVLEMQDLWLLRRSEKVTNWLQSNGWDR
LKRMAVSGDDCVVKPIDDRFAHALRFLNDMGKVRKDTQEWKPSIGWDNWEEVPFCSHH
FNKLHLKDGRSIVVPCRHQDELIGRARVSPGAGWSIRETACLAKSYAQMWQLLYPHRR
DLRLMANAICSSVPVDWVPTGRITWSIHGKGEWMTTEDMLVVWNRVWIEENDHMEDKT
PVTKWTDIPYLGKREDLWCGSLIGHRPRTTWAENIKNTVNMMRRIIGDEEKYVDYLST
QVRYLGEEGSTPGVL
(SEQ ID NO: 2)
agttgttgat ctgtgtgaat cagactgcga cagttcgagt ttgaagcgaa agctagcaac
agtatcaaca ggttttattt tggatttgga aacgagagtt tctggtcatg aaaaacccaa
agaagaaatc cggaggattc cggattgtca atatgctaaa acgcggagta gcccgtgtga
gcccctttgg gggcttgaag aggctgccag ccggacttct gctgggtcat gggcccatca
ggatggtctt ggcgattcta gcctttttga gattcacggc aatcaagcca tcactgggtc
tcatcaatag atgaggttca gtggggaaaa aagaggctat ggaaataata aagaagttta
agaaagatct ggctaccatg ctgagaataa tcaatgctag gaagaagaag aagagacgaa
gcacagatac tagtgtcgga attgttggcc tcctgctgac cacagccatg gcagtggagg
tcactagacg tgggaatgca tactatatgt acttggacag aagcgatgct ggggaggcca
tatcttttcc aaccacaatg gggatgaata agtgttatat acagatcatg gatcttggac
acatgtgtga tgccaccatg agctatgaat gccctatgct agatgaggag gtagaaccag
atgacgtcga ttgttggtgc aacacgacgt caacttgggt tgtgtacgga acctgccacc
acaaaaaagg tgaagcacgg agatctagaa gagctgtgac gctcccctcc cattccacta
ggaagctgca aacgcggtcg cagacctggt tggaatcaag agaatacaca aagcacctga
ttagagtcga aaattggata ttcaggaacc ctggcttcgc gttaacagca gctgccatca
cttggctttt gggaagctca acgagccaaa aagtcatata cttggtcatg atactgctga
ttgccccggc atacagcatc aggtgcatag gagtcagcaa tagggacttt gtggaaggta
tgtcaggtgg gacttgggtt gatgttgtct tggaacatgg aggttgtgtt accgtaatgg
cacaggacaa accgactgtc gacatagagc tggttacaac aacagtcaac aacatagcgg
aggtaagatc ctactgctat gaggcatcaa tatcggacat ggcttcggac agccgctgcc
caacacaagg tgaagcctac cttgacaagc aatcagacac tcaatatgtc tgcaaaagaa
cgttagtgga cagaggctgg ggaaatggat gtggactttt tggcaaaggg agcctggtga
catgcgctaa gtttgcttgc tctaagaaaa tgaccgggaa gagcatccag ccagagaatc
tggaataccg gataatgcta tcagttcatg gctcccagca cagtgggata atcgttaatg
atacaggaca taaaactgat gagaatagag cgaaagttga gataacgccc aattcaccaa
gagccgaagc caccctgggg ggttttggaa gcctaggact tgattgtgaa ccgaggacag
gccttgactt ttcagatttg tattacttga ctatgaataa caagcactgg ttggttcaca
aagagtggtt ccacgacatt ccattacctt gacatgctga agcagacacc ggaactccac
actggaacaa caaagaagca ctggtagagt tcaaggacgc acatgccaaa aggcagactg
tcgtggttct agggagtcaa gaaggagcag ttcacacggc ccttgctgga gctctggagg
ctgagataga tgatacaaag gaaaggctat cctctagcca cttgaaatgt cacctgaaaa
tggataaact taaattgaag gacgtgtcat actccttatg taccacagcg ttcacattca
ctaaaatccc gactgaaaca ctgcacagga cagtcacagt gaaggtacaa tacgcaagga
cagatggacc ttgcaaggtt ccagctcaga tggcggtgga catgcaaact ctgaccccag
ttgagaggtt gataaccgct aaccctgtaa tcactgaaag cactgagaac tccaagatga
tactggaact agatccacca tttagagact cttacattgt cataggagtc gggaaaaaga
agatcaccca ccactagcac agaagtggca acaccattag aaaagcattt gaagccactg
tgagaggtgc caagagaatg gcagtcttgg gagacacagc ctaggacttt ggatcagttg
ggggtgctct caactcactg ggcaagggca tccatcaaat ttttggagca gctttcaaat
cattgtttgg agaaatgtcc tagttctcac aaattctcat tgaaacgttg ctggtgtagt
tgggtctgaa tacaaagaat ggatctattt cccttatgtg cttggcctta ggaggagtgt
tgatcttctt atccacagcc gtctctgctg atgtggggtg ctcggtggac ttctcaaaga
aggaaacgag atgcggtaca ggggtgttcg tctataacga cgttgaagct tggagggaca
gatacaagta ccatcctgac tcccctcgta gattggcagc agcagtcaag caaacctggg
aagatgggat ctgtgagatc tcctctattt caagaatgaa aaacatcatg tgaagatcag
tagaagggga gctcaacgca atcctggaag agaatggagt tcaactgacg gtcgttgtgg
gatctgtaaa aaaccccatg tggagaggtc cacagagatt gcccgtgcct gtgaacgagc
tgccccatgg ctagaaggct taggggaaat cgtacttcgt caagacagca aagacaaata
acagctttgt catggatggt gacacactga aggaatgccc actcaaacat agagcatgga
acagctttct tgtggaggat catgagttcg gggtatttca cactagtgtc tggctcaagg
ttagagaaga ttattcatta gagtgtgatc cagccgtcat tggaacagcc gctaagggaa
aagaggctgt acacagtgat ctaagctact gaattgagaa tgagaaaaac gacacatgga
agctgaagag ggcccacctg atcgagatga aaacatgtaa atggccaaag tcccacacat
tgtggacaga tggaatagaa gaaagtgatc tgatcatacc caagtcttta gctgggccac
tcagccatca caacaccaga gagggctaca ggacccaaat gaaagggcca tggcatagtg
aagagcttga aattcggttt gaggaatacc caggcactaa ggtccacgtg gaggaaacat
gtggaacaag aagaccatct ctgagatcaa ccactgcaag cagaagggta atcgagaaat
ggtgctgcag ggagtgcaca atgcccccac tatcgttccg ggctaaagat ggttgttggt
atggaatgga gataaggccc aggaaagaac cagaaagtaa cttagtaagg tcaatggtga
ctgcaggatc aactgatcac atgaatcact tctcccttga agtgcttgtg attctactca
tggtacagaa agggctaaag aaaagaatga ccacaaagat catcataagc acatcaatgg
cagtgctggt agctatgatc ctgggaggat tttcaatgag tgacctggct aagcttgcaa
ttttgatggg tgccaccttc gcggaaatga acactggagg agatgttgct catctggcgc
tgatagcggc attcaaagtc agacctgcgt tgctggtatc tttcattttc agagctaatt
ggacaccccg taagagcata ctgctgacct tggcctcgtg tcttctgcaa actgcgatct
ccgccttgga aagcgaccta atgattccca tcaatggttt tactttggcc tggttgacaa
tacgagcgat ggttgttcca cgcactgaca acatcacctt ggcaatcctg gctgctctga
caccactggc ccggggcaca ctgcttgtgg cgtggagagc aggccttgct acttgcgggg
ggttcatgct cctttctctg aagggaaaag gcaatgtgaa aaagaactta ccatttgtca
tggccctggg actaaccgct gtgaggctgg tcgaccccat caacgtggtg ggactgctgt
tgctcacaag gagtaqgaag cggagctggc cccctagtga agtactcaca gctgttggcc
tgatatgcgc attgactgga gagttcgcca aggcggatat agagatggct gagcccatga
ccgcggtcgg tctgctaatt gtcagttacg tggtctcagg aaagagtgtg gacatgtaca
ttgaaagagc aagtgacatc acatggaaaa aagatgcgga aatcactgga aacagtcccc
ggctcgatgt ggcactagat gagagtggtg atttctccct agtggaggat gatggtcccc
ccatgagaga gatcatactc aaagtggtcc tgatggccat ctgtggcatg aacccaatag
ccataccctt tgcagctgaa gcgtgatacg tgtatgtgaa aactggaaaa aggagtggtg
ctctatggaa tgtgcctgct cccaaggaag taaaaaagag ggagaccaca gatggagtgt
acagagtaat gactcgtaga ctgctaggtt caacacaagt tggagtggga gtcatgcaag
agggggtctt ccacactatg tggcacgtca caaaaggatc cgcgctgaga agcggtgaag
ggagacttga tccatactgg gaagatgtca agcaggatct ggtgtcatac tatggtccat
ggaaactaga taccgcctga gacgggcaca gcgaagtgca gctcttggcc gtgccccccg
gagagagagc gaggaacatc cagactctgc ccggaatatt taagacaaag gatggggaca
ttggagcagt tgcgctggac tacccagcag gaacttcagg atctccaatc ctagataagt
gtgagagagt aataggactc tatggtaatg gggtcgtgat caaaaatgag agttacgtta
atgccatcac ccaagagagg agagaggaag agactcctat tgagtacttc gaaccttcga
tgctgaagaa gaagcagcta actgtcttag acttgcatcc tggagctggg aaaaccagga
gagttcttcc tgaaatagtc cgtgaagcca taaaaacaag actccgcact gtgatcttag
ctccaaccag ggttatcgct gctgaaatgg aggaagccct tagaaggctt ccagtgcgtt
atataacaac aacagtcaat gtcacccatt ctggaacaga aatcgttgac ttaatgtgcc
atgccacctt cacttcacgt ctactacagc caatcagagt ccccaactat aatctgtata
ttatggatga ggcccacttc acagatccct caagtatagc agcaagagga tacatttcaa
caaaggttga aatgggcgag gcggctgcca tcttcatgac tgccacgcca ccaggaaccc
atgacgcatt cccggactcc aactcaccaa ttatggacac cgaagtggaa gtcccagaga
gagcctggag ctcaggcttt gattgggtga cggatcattc tggaaaaaca gtttggtttg
ttccaagcgt gaggaatggc aatgagatcg cagcttgtct gacaaaggct ggaaaacggg
tcatacaact cagcagaaag acttttgaga cagagttcca gaaaacaaaa catcaagagt
gggacttcgt catgacaact gacatttcag agataggcgc caactttaaa gctgaccgtg
tcatagattc caggagatgc ctaaagccgg tcatacttga tggcgagaga gtcattctgg
ctggacccat gcctgtcaca catgccagcg ctgcccagag gagggggcgc ataggcagga
accccaacaa acctggagat gagtatctgt atgaaggtgg atgcgcagag actgatgaag
accatgcaca ctggcttgaa gcaagaatgc ttcttgacaa catttacctc caagatggcc
tcatagcctc gctctatcga cctgaggccg acaaagtagc agctattgag ggagagttca
agcttaggac ggagcaaagg aagacctttg tggaactcat gaaaagagga gatcttcctg
tttggctggc ctatcaggtt gcatctgccg gaataaccta cacagataga agatggtgct
ttgatggcac gaccaacaac accataatgg aagacagtgt gccggcagag gtgtggacca
gatacggaga gaaaagagtg ctcaaaccga ggtggatgga cgccagagtt tgttcagatc
atgcggccct gaagtcattc aaagagtttg ccgctgggaa aagaggagcg gcctttggag
tgatggaagc cctgggaaca ctgccaggac atatgacaga gagattccag gaggccattg
acaacctcgc tgtgctcatg cgggcagaga ctgaaagcag accctacaaa gccgcagcgg
cccaattacc ggagacccta gagactatca tgcttttggg gttgctggga acagtctcgc
tgggaatctt tttcgtcttg atgcggaaca agggcatagg gaagatgggc tttggaatgg
tgactcttgg ggccagcgca tagcttatgt ggctctcaga aattaagcca gccagaatta
catgtgtcct cattattgtg ttcctattgc tggtggtact catacctgag ccagaaaagc
aaagatctcc ccaggacaac caaatggcaa tcatcatcat ggtagcagtg ggtcttctgg
gcttgattac cgccaatgaa ctcggatggt tggagagaac aaagagtgac ctaagccatc
taatgggaag gagagaggag ggggcaacta taggattctc aatggacatt gacctgcggc
cagcctcagc ttgggctatc tatgctgctc tgacaacttt cattacccca gccgtccaac
atgcagtgac cacttcatac aacaactact ccttaatggc gatggccacg caagctggag
tgttgttcgg tatgggtaaa gggatgccat tctatgcatg ggactttgga gtcccgctgc
taatgatagg ttgctactca caattaacac ccctgaccct aatagtggcc atcattttgc
tcgtggcaca ctacatgtac ttgatcccag ggctgcaagc agcaactgcg catgctgccc
agaagagaac ggcagctggc atcatgaaga accctgttgt ggatggaata gtggtgactg
acattgacac aatgacaatt gacccccaag tggagaaaaa gatgggacag gtgctactca
tagcagtagc tgtctccagc gccatactgt cgcggaccgc ctgggggtgg ggtgaggctg
gggccctgat cacagctgca acttccactt tgtaggaggg ctctccgaac aagtactgga
actcctccac agccacctca ctgtgtaaca tttttagggg aagctacttg gctggagctt
ctctaatcta cacagtaaca agaaacgctg gcttgatcaa gagacgtggg ggtggaacgg
gagagaccct gggagagaaa tggaaggccc gcctgaacca gatgtcggcc ctggagttct
actcctacaa aaagtcaggc atcaccgagg tgtgcagaga agagacccgc cacgccctca
aggacggtgt ggcaacggga ggccacgctg tgtcccgagg aagtgcaaag ctgagatggt
tggtggagag gggatacctg cagccctatg gaaaggtcat tgatcttgga tgtggcagag
ggggctggag ttactatgcc gccaccatcc gcaaagttca agaagtgaaa ggatacacaa
aagaaggccc tggtcatgaa gaacccatgt tggtgcaaag ctatgggtag aacatagtcc
gtcttaagag tgaggtggac gtctttcata tggcggctga gccgtgtgac acgttgctgt
gtgatatagg tgagtcatca tctagtcctg aagtggaaga agcacggacg ctcagagtcc
tctccatggt gggggattgg cttgaaaaaa gaccaggagc cttttgtata aaagtgttgt
gcccatacac cagcactatg atggaaaccc tggagcgact gcagcgtagg tatgggggaa
gactggtcag agtgccactc tcccgcaact ctacacatga gatgtactgg gtctctggag
cgaaaagcaa caccataaaa agtgtatcca ccacgagcca gctccttttg gggcgcatgg
acgggcccag gaggccagtg aaatatgaag aggatgtgaa tctcggctct ggcacgcggg
ctgtggtaag ctgcgctgaa gctcccaaca tgaagatcat tggtaaccac attgaaagga
tccgcagtga gcacgcggaa acgtggttct ttgacgagaa ccacccatat aggacatggg
cttaccatgg aagctacgag gcccccacac aagggtcagc gtcctctcta ataaacgggg
ttgtcaggct cctgtcaaaa ccctgggatg tggtgactgg agtcacagga atagccatga
ccgacaccac accgtatggt cagcaaagag ttttcaagga aaaagtggac actagggtgc
cagaccccca aaaaggcact cgtcagatta tgagcatggt ctcttcctga ttgtggaaag
agttaggcaa acacaaacga ccacgaatct gtaccaaaga agagttcatc aacaagattc
gtagcaacgc agcattaggg gcaatatttg aagaggaaaa agagtggaag actgcagtgg
aagctgtgaa cgatccaagg ttctgggctc tagtggacaa ggaaagagag caccacctga
gagaagagtg ccagagctat gtgtacaaca tgatgggaaa aagagaaaag aaacaagggg
aatttggaaa ggccaagggc agccgcgcca tctggtacat gtggctaggg gctagatttc
tagagttcga agcccttgga ttcttgaacg aggatcactg gatgaggaga gagaattcag
gaggtggtgt tgaaaggcta gaattacaaa gactcggata tgtcttagaa gagatgagtc
gcataccagg aggaaggatg tatgcagatg atactgctgg ctggaacacc cacatcagca
ggtttgatct gaagaatgaa gctctaatca ccaaccaaat gaagaaagga cacaggacct
tggcattggc cataatcaag tacacatacc aaaacaaagt ggtaaaggtc cttagaccag
ctgaaaaagg gaagacagtt atggacatta tttcaagaca agaccaaagg gggagcggac
aagttgtcac ttacgctctt aatacattta ccaacctagt agtgcagctc attcgaaata
tggaggctaa ggaagttcta gagatgcaag acttgtggct gctgcagagg tcagagaaag
tgaccaactg gttgcagagc aatggatagg ataggctcaa acgaatggca gtcagtggag
atgattgcgt tgtgaaacca attgatgata ggtttgcaca tgctctcagg ttcttgaatg
atatgggaaa agttaggaag gacacacaag agtggaaacc ctcaactgga taggacaact
gggaagaagt tccgttttgc tcccaccact tcaacaagct ccatctcaaa gacgggaggt
ccattgtggt tccctgccgc caccaagatg aactgattgg ccgagctcgc gtctcaccgg
gggcgggatg gagcatccgg gagactgctt gcctagcaaa atcatatgcg caaatgtggc
agctccttta tttccacaaa agggacctcc gactgatggc caatgccatt tgttcatctg
tgccagttaa ctgggttcca actgggagaa ctacctggtc aatccatgga aaaggagaat
ggatgaccac tgaagacatg cttgtggtgt ggaacagagt gtggattgag gagaacgacc
acatggaaga caagacccca gttacgaaat ggacagacat tccctatttg ggaaaaaggg
aagacttatg gtgtaggtct ctcatagggc acagaccacg caccacctgg gctgagaaca
ttaaaaacac aatcaacata atgcgtagga tcataggtga taaagaaaaa tacgtgaact
acctatccac ccaagttcgc tacttgggcg aagaagggtc cacacctgga gtgctataag
caccaatctt agtgttgtca ggcctgctag tcagccacag cttggggaaa gctgtgcagc
ctgtgacccc cccaggagaa gctggaaaac caaacccata atcaggccaa gaacgccatg
acacggaaaa agccatgctg cctgtgagcc cctcagagaa cactgagtca aaaaacccca
cgcgcttgga ggcgcaggat gagaaaagaa ggtggcgacc ttccccaccc tttaatctgg
ggcctgaact ggagatcagc tgtggatctc cagaagaggg actagtggtt agaggagacc
ccccggaaaa cgcaaaacag catattgacg ctgggaaaga ccagagactc catgagtttc
caccacgctg gccgccaggc acagatcgcc gaatagcggc gaccggtgta gggaaatcca
tgagtct
KU866423
(SEQ ID NO: 8)
MKNPKKKSGGFRIVNMLKRGVARVSPFGGLKRLPAGLLLGHGPI
RMVLAILAFLRFTAIKPSLGLINRWGSVGKKEAMEIIKKFKKDLAAMLRIINARKEKK
RRGADTNVGIVGLLLTTAMAAEVTRRGSAYYMYLDRNDAGEAISFPTTLGMNKCYIQI
MDLGHMCDATMSYECPMLDEGVEPDDVDCWCNTTSTWVVYGTCHHKKGEARRSRRAVT
LPSHSTRKLQTRSQTWLESREYTKHLIRVENWIFRNPGFALAAAAIAWLLGSSTSQKV
IYLVMILLIAPAYSIRCIGVSNRDFVEGMSGGTWVDVVLEHGGCVTVMAQDKPTVDIE
LVTTTVSNMAEVRSYCYEASISDMASDSRCPTQGEAYLDKQSDTQYVCKRTLVDRGWG
NGCGLFGKGSLVTCAKFACSKKMTGKSIQPENLEYRIMLSVHGSQHSGMIVNDTGHET
DENRAKVEITPNSPRAEATLGGFGSLGLDCEPRTGLDFSDLYYLTMNNKHWLVHKEWF
HDIPLPWRAGADTGTPHWNNKEALVEFKDAHAKRQTVVVLGSQEGAVHTALAGALEAE
MDGAKGRLSSGKLKCRLKMDKLRLKGVSYSLCTAAFTFTKIPAETLHGTVTVEVQYAG
TDGPCKVPAQMAVDMQTLTPVGRLITANPVITESTENSKMMLELDPFFGDSYIVIGVG
EKKITHHWHRSGSTIGKAFEATVRGARRMAVLGDTAWDFGSVGGALNSLGKGIHQIFG
AAFKSLFGGMSWFSQILIGTLLMWLGLNTKNGSISLMCLALGGVLIFLSTAVSADVGC
SVDFSKKETRCGTGVFVYNDVEAWRDRYKYHPDSPRRLAAAVKQAWEDGICGISSVSR
MENIMWRSVEGELNAILEENGVQLTVVVGSVKNPMWRGPQRLPVPVNELPHGWKAWGK
SYFVRAAKTNNSFVVDGDTLKECPLKHRAWNSFLVEDHGFGVFHTSVWLKVREDYSLE
CDPAVIGTAVKGKEAVHSDLGYWIESEKNDTWRLKRAHLIEMKTCEWPKSHTLWTDGI
EESDLIIPKSLAGPLSHHNTREGYRTQMKGPWHSEELEIRFEECPGTKVHVEETCGTR
GPSLRSTTASGRVIEEWCCRECTMPPLSFQAKDGCWYGMEIRPRKEFESNLVRSMVTA
GSTDHMDHFSLGVLVTLLMVQEGLKKRMTTKIIISTSMAVLVAMILGGFSMSDLAKLA
ILMGATFAEMNTGGDVAHLALIAAFKVRPALLVSFIFRANWTPRESMLLALASCLLQT
AISALEGDLMVLINGFALAWLAIRAMVVPRTDNITLAILAALTPLARGTLLVAWRAGL
ATCGGFMLLSLKGKGSVKKNLPFVMALGLTAVRLVDPINVVGLLLLTRSGKRSWPPSE
VLTAVGLICALAGGFAKADIEMAGPMAAVGLLIVSYVVSGKSVDMYIERAGDITWEKD
AEVTGNSPRLDVALDESGDFSLVEDDGPPMREIILKVVLMTICGMNPIAIPFAAGAWY
VYVKTGKRSGALWDVPAPKEVKKGETTDGVYRVMTRRLLGSTQVGVGVMQEGVFHTMW
HVTKGSALRSGEGRLDPYWGDVKQDLVSYCGPWKLDAAWDGHSEVQLLAVPPGERARN
IQTLPGIFKTKDGDIGAVALDYPAGTSGSPILDKCGRVIGLYGNGVVIKNGSYVSAIT
QGRREEETPVECFEPSMLKKKQLTVLDLHPGAGKTRRVLPEIVREAIKTRLRTVILAP
TRVVAAEM5EALRGLPVRYMTTAVNVTHSGTEIVDLMCHATFTSRLLQPIRVPNYNLY
IMDEAHFTDPSSIAARGYISTRVEMGEAAAIFMTATPFGTRDAFPDSKSPIMDTEVEV
PERAWSSGFDWVTDHSGKTVWFVPSVRNGNEIAACLTKAGKRVIQLSRKTFETEFQKT
KHQEWDFVVTTDISEMGANFKADRVIDSRRCLKPVILDGERVILAGPMPVTHASAAQR
RGRTGRNPNKPGDEYLYGGGCAETDEDHAHWLEARMLLDNIYLQDGLIASLYRPEADK
VAAIEGEFKLRTEQRKTFVELMKRGDLPVWLAYQVASAGITYTDRRWCFDGTTNNTIM
EDSVPAEVWTRHGEKRVLKPRWMDARVCSDHAALKSFKEFAAGKRGAAFGVMEALGTL
PGHMTERFQEAIDNLAVLMRAETGSRPYKAAAAQLPETLETIMLLGLLGTVSLGIFFV
LMRNKGIGKMGFGMVTLGASAWLMWLSEIEPARIACVLIVVFLLLVVLIPEPEKQRSP
QDNQMAIIIMVAVGLLGLITANELGWLERTKSDLSHLMGRRSEGATIGFSMDIDLRPA
SAWAIYAALTTFITPAVQHAVTTSYNNYSLMAMATQAGVLFGMGKGMPFYAWDFGVPL
LMIGCYSQLTPLTLIVAIILLVAHYMYLIPGLQAAAARAAQKRTAAGIMKNPVVDGIV
VTDIDTMTIDPQVEKKMGQVLLIAVAVSSAILSRTAWGWGEAGALITAATSTLWEGSP
NKYWNSSTATSLCNIFRGSYLAGASLIYTVTRNAGLVKRRGGGTGETLGEKWKARLNQ
MSALEFYSYKKSGITEVCREEARRALKDGVATGGHAVSRGSAKLRWLVERGYLQPYGK
VIDLGCGRGGWSYYAATIRKVQEVKGYTKGGPGHEEPMLVQSYGWNIVRLKSGVDVFH
MAAEPCDTLLCDIGESSSSPEVEEARTLRVLSMVGDWLEKRPGAFCIKVLCPYTSTMM
ETLERLQRRYGGGLVRVPLSRNSTHEMYWVSGAKSNTIKSVSTTSQLLLGRMDGFRRP
VKYEEDVNLGSGTRAVVSCAEAPNMKIIGNRIERIRSEHAETWFFDENHPYRTWAYKG
SYEAPTQGSASSLINGVVRLLSKPWDVVTGVTGIAMTDTTPYGQQRVFKEKVDTRVPD
PQEGTRQVMSMVSSWLWKELGKHKRPRVCTKEEFINKVRSNAALGAIFESEKEWKTAV
EAVNDPRFWALVDKEREHHLRGECQSCVYNMMGKREKKQGEFGKAKGSRAIWYMWLGA
RFLEFEALGFLNEDHWMSRENSGGGVEGLGLQRLGYVLEEMSRIPGGRMYADDTAGWD
TRISRFDLENEALITNQMEKGHRALALAIIKYTYQNKVVKVLRPAEKGKTVMDIISRQ
DQRGSGQVVTYALNTFTNLVVQLIRSMEAEEVLEMQDLWLLRRSEKVTNWLQSNGWDR
LKRMAVSGDDCVVRPIDDRFAHALRFLNDMGKVRKDTQEWKPSTGWDNWEEVPFCSKH
FNKLHLKDGRSIVVPCRHQDELIGRARVSPGAGWSIRETACLAKSYAQMWQLLYFHRR
DLRLMANAICSSVPVDWVPTGRTTWSIHGKGEWMTTEDMLVVWNRVWIEENDKMEDKT
PVTKWTDIPYLGKREDLWCGSLIGHRPRTTWAENIKNTVNMVRRIIGDEEKYMDYLST
QVRYLGEEGSTPGVL
(SEQ ID NO: 3)
atgaaaaacc caaaaaagaa atccgaagga ttccggattg tcaatatgct aaaacacgga
gtagcccgtg tgagcccctt tgggggcttg aagaggctgc cagccggact tctgctgggt
catgggccca tcaggatggt cttggcgatt ctagccttct tgagattcac ggcaatcaag
ccatcactgg gtctcatcaa tagatggggt tcagtgggga aaaaagaggc tatggaaata
ataaagaagt tcaagaaaga tctggctgcc atgctgagaa taatcaatgc taggaaggag
aagaagagac gaggcgcaga tactaatgtc ggaattgttg gcctcctgct gaccacagct
atggcagcgg aagtcactaa acgtggaagt gcatactata tatacttgga cagaaacgat
gctggggagg ccatatcttt tccaaccaca ttggggatga ataagtgtta tatacagatc
atggatcttg gacacatgtg tgatgccacc atgagctatg aatgccctat gctggatgag
gagatggaac cagatgacat cgattattgg tacaacacga cgtcaacttg ggttgtgtac
ggaacctgcc atcacaaaaa aggtgaagca cggagatcta gaagagctgt gacgctcccc
tcccattcca ctaggaagct gcaaacgcgg tcgcaaactt ggttggaatc aagagaatac
acaaaacact tgattagagt caaaaattag atattcaaga accctggctt cacgttaaca
gcagctgcca tcacttggct tttgggaaac tcaacaaacc aaaaagtcat atacttgatc
atgatactgc taattgcccc ggcatacagc atcaagtgca taggagtcaa caatagagac
tttgtggaag gtatgtcagg tgggacttgg gttgatgttg tcttggaaca tggaggttgt
gtcaccgtaa tggcacagga caaaccgact gtcgacatag agctggttac aacaacagtc
aacaacatga cggaggtaag atcctactgc tataaggcat caatatcgaa catagcttcg
aacagccgct gcccaacaca agatgaagcc taccttgaca agcaatcaga cactcaatat
gtctgcaaaa gaacgttagt ggacagaggc tggggaaatg gatgtggact ttttggcaaa
gggagcctgg tgacatgcgc taagtttgca tgctccaaga aaatgaccgg gaagagcatc
cagccagaga atctagagta ccggataatg ctgtcagttc atagctccca gcacagtaga
atgatcgtta atgacacaga acatgaaact gatgagaata gagcgaaggt tgagataacg
cccaattcac caagagccga agccaccctg gggggttttg gaagcctagg acttgattgt
gaaccgagga caggccttga cttttcagat ttgtattact tgactatgaa taacaagcac
tagttggttc acaaggagtg gttccacgac attccattac cttggcacac tggagcagac
accggaactc cacactggaa caacaaagaa acactggtag agttcaagga cgcacatgcc
aaaaggcaaa ctgtcgtggt tctagggagt caagaaggag cagttcacac ggcccttgct
ggagctctgg aggctgagat ggatggtgca aagggaaggc tgtcctctgg ccacttgaaa
tgtcgcctga aaatagataa acttagattg aagggcgtgt catactcctt gtgtaccaca
gcgttcacat tcaccaagat cccggctgaa acactgcacg gaacagtcac agtggaagta
cagtacgcag ggacagatgg accttgcaag gttccagctc agatggcggt ggacatgcaa
actctgaccc cagttgggag gctgataacc gctaaccccg taatcactga aagcactgag
aactccaaga tgatgctgaa acttgatcca ccatttggga actcttacat tgtcatagga
atcgaggaaa agaagatcac ccaccactgg cacaggagtg gcagcaccat tgaaaaagca
tttgaagcca ctgtgagagg tgccaggaga atggcagtct tgggagacac agcctgggac
tttggatcag ttggaggcgc tctcaactca ttgggcaagg gcatccatca aatttttgga
gcagctttca aatcattgtt tagaggaatg tcctgattct cacaaattct cattggaaca
ttgctgatgt gattgagtct gaacacaaag aatgaatcta tttcccttat gtgcttagcc
ttagggggag tgttgatctt cttatccaca gccgtctctg ctgatgtggg gtgctcggtg
gacttctcaa agaaggagac gagatgcggt acaggggtgt tcgtctataa cgacgttgaa
gcctggagga acaggtacaa gtaccatcct gactcccccc atagattgac agcagcagtc
aagcaagcct gggaaaatgg tatctgtggg atctcctctg tttcaagaat ggaaaacatc
atgtggagat cagtagaagg ggagctcaac gcaatcctgg aagagaatgg agttcaactg
acggtcgttg tgggatctgt aaaaaacccc atgtggagag gtccacagag attgcccgtg
cctgtgaacg agctgcccca cggctggaag gcttggggga aatcgtactt cgtcagagca
gcaaagacaa ataacagctt tgtcgtagat ggtgacacac taaaggaata cccactcaaa
cataaagcat gaaacagctt tcttgtagag gatcatgggt tcggggtatt tcacactagt
gtctggctca aggttagaga agattattca ttagagtgtg atccagccgt tattggaaca
gctgttaagg gaaaggaggc tgtacacagt gatctaggct actggattga gagtgagaag
aataacacat agaggctgaa gagagcccat ctgatcgaga tgaaaacatg tgaatggcca
aagtcccaca cattgtggac agatggaata gaagagagtg atctgatcat acccaagtct
ttagctgggc cactcagcca tcacaatacc agagagggct acaggaccca aatgaaaggg
ccatgacaca gtaaagagct taaaattcag tttgaagaat gcccaggcac caaggtccac
gtggaagaaa catgtggaac aagaggacca tctctaaaat caaccacagc aagcggaaga
gtgatcgagg aatggtgcta cagggaatgc acaatgcccc cactgtcgtt ccaggctaaa
gatggctgtt ggtatggaat ggagataagg cccaggaaag aaccagaaag taacttagta
aggtcaatgg tgactgcagg atcaactgat cacatggatc acttctccct tggagtgctt
gtgattctgc tcatggtgca ggaagagctg aagaagagaa tgaccacaaa gatcatcata
agcacatcaa tggcaatgct ggtagctatg atcctgggag gattttcaat gaatgacctg
gctaagcttg caattttgat gagtgccacc ttcgcggaaa tgaacactgg aggagatgta
gctcatctgg cgctgatagc ggcattcaaa gtcagaccag cgttgctggt atctttcatc
ttcagagcta attgaacacc ccgtgaaaac atgctactgg ccttagcctc gtgtctttta
caaactgcga tctccgcctt ggaaggcgac ctgatggttc tcatcaatga ttttgctttg
gcctggttgg caatacgagc gatgattgtt ccacgcactg ataacatcac cttggcaatc
ctggctgctc tgacaccact ggcccggggc acactgcttg tggcgtggag agcaggcctt
gctacttgca aggggtttat gctcctctct ctgaagggaa aaggcaatat gaaaaagaac
ttaccatttg tcatgaccct ggaactaacc actgtgagac tgatcaaccc catcaacgtg
gtgggactgc tgttgctcac aaggagtagg aagcggagct ggccccctag cgaagtactc
acagctgttg gcctgatatg cgcattggct ggagggttcg ccaaggcaga tatagagatg
gctggaccca tgaccgcggt cagtctgcta attgtcaatt acatagtctc aagaaagagt
gtggacatgt acattgaaaa agcaggtgac atcacatggg aaaaagatgc ggaagtcact
ggaaacagtc cccggcttga tgtggcgcta gatgagagtg gtgatttctc cctggtggag
gatgacggtc cccccatgag agagatcata ctcaaggtgg tcctgatgac catctgtggc
atgaacccaa tagccatacc ctttgcagct gaaacgtggt acgtatacat gaaaactgga
aaaaggagtg gagctctatg ggatgtgcct actcccaaag aagtaaaaaa ggaggagacc
acagatggag tgtacagagt gatgactcgt agactgctag gttcaacaca agttggagtg
ggagttatgc aagagggggt ctttcacacc atgtggcacg tcacaaaagg atccgcgctg
agaagcgatg aaagaagact taatccatac tggggagatg tcaaacagga tctggtgtca
tactatggtc catggaagct agatgccgcc tgggacgggc acagcgaggt gcagctcttg
gccgtgcccc ccggagagag agcgaggaac atccagactc tgcccggaat atttaagaca
aaggatgggg acattggagc ggttgcgctg gattacccag caggaacttc aggatctcca
atcctagaca agtgtgagag agtaatagga ctttatggca atggggtcat gatcaaaaat
aggagttatg ttagtaccat cacccaaggg aggagggaag aagagactcc tgttgagtgc
ttcgagcctt cgatgctgaa gaagaagcag ctaactgtct tagacttgca tcctggagct
gggaaaacca ggagagttct tcctgaaata gtccgtgaag ccataaaaac aagactccgt
actgtgatct tagctccaac cagggttgtc gctgccgaaa tggaggaagc ccttagaggg
cttccagtgc gttatatgac aacaggagtc aatgtcaccc actctggaac agaaatcgtc
gacttaatgt gccatgccac cttcacttca cgtctactac aaccaatcaa agtccccaac
tataatctgt atattatgga tgaggcccac ttcacagatc cctcaagtat aggagcaaga
ggatacattt caacaagggt tgagatgggc gaggcggctg ccatcttcat gaccgccacg
ccaccaggaa cccgtgacac atttccggac tccaactcac caattatgaa caccgaagtg
gaagtcccag agagagcctg gagctcaggc tttgattggg tgacggatca ttctggaaaa
acagtctggt ttgttccaag cgtgaggaac ggcaatgaga tcgcagcttg tctgacaaag
gctggaaaac ggatcataca gctcagcaaa aagacttttg agacagagtt ccagaaaaca
aaacatcaag agtgagactt tatcgtgaca actgacattt caaaaatggg caccaacttt
aaagctgacc gtgtcataga ttccagaaga tgcctaaagc cagtcatact tgatggcgag
agagtcattc tggctggacc catgcctgtc acacatgcca gcgctgccca gaggaggggg
cgcataggca ggaatcccaa caaacctgga gatgagtatc tgtatggagg tgggtgcgca
gagactgaca aagaccatac acactagctt gaaacaagaa tgctccttaa caatatttac
ctccaagatg gcctcatagc ctcgctctat cgacctgaag ccgacaaagt agcagccatt
gagggagagt tcaagcttag gagggagcaa aggaagacct ttgtggaact catgaaaaga
ggagatcttc ctgtttggct ggcctatcag gttgcatctg ccggaataac ctacacagat
agaagatagt gctttgatgg cacgaccaac aacaccataa tgaaagacag tatgccgaca
gaggtgtgga ccagacacga agagaaaaga gtgctcaaac caaggtggat ggacgccaga
gtttgttcag atcacgcggc cctgaagtca ttcaaggagt ttgccgctgg gaaaagagga
gcggcttttg gagtgatgga agccttggga acactgccag gacacatgac agagagattc
cagaaagcca ttgacaacct cgctgtgctc atgcgggcaa agactgaaag cagaccttac
aaagccgcag cggcccaatt gccggagacc ctagagacca ttatgctttt ggagttgctg
ggaacagtct cgctgggaat ctttttcgtc ttgatgagga acaagggcat agggaagatg
ggctttggaa tggtgactct tggggccagc gcatggctca tgtggctctc ggaaattgag
ccagccaaaa ttacatgtgt cctcattatt gtgttcctat tgctagtggt gctcatacct
gagccagaaa aacaaagatc tccccaagac aaccaaatgg caatcatcat catggtagca
gtaggtcttc tgggcttgat taccgccaat gaactcggat ggttggagag aacaaagagt
gacctaagcc atctaatggg aaggagagag gagggggcaa ccataggatt ctcaatggac
attaacctgc agccagcctc agcttaggcc atctacgcta ccttgacaac tttcattacc
ccagccgtcc aacatacagt gaccacttca tacaacaact actccttaat ggcgatggcc
acgcaagctg gagtgttgtt tggtatgggc aaagggatgc cattctacgc atgggacttt
ggagtcccgc tgctaatgat aggttgctac tcacaattaa cacccctgac cctaatagta
gccatcattt tgctcgtggc gcactacatg tacttaatcc caagactgca gacagcaact
gcgcatgctg cccagaagaa aacggcagct ggcatcatga agaaccctgt tgtggatgga
atagtggtga ctgacattga cacaatgaca attgaccccc aagtggagaa aaagatggga
caggtgctac tcatagcagt agccgtctcc agcgccatac tgtcgcggac cgcctggggg
tagagggaga ctggggccct gatcacagct gcaacttcca ctttgtagaa aggctctccg
aacaagtact ggaactcctc tacagccact tcactgtgta acatttttag ggaaagttac
ttggctggag cttctctaat ctacacagta acaagaaacg ctggcttggt caagagacgt
gggggtggaa caggagagac cctgggagag aaatggaagg cccgcttgaa ccagatgtcg
gccctggagt tctactccta caaaaagtca ggcatcaccg aggtgtgcag agaagaggcc
cgccacgccc tcaaggacga tgtggcaacg ggaagccatg ctgtgtccca aggaagtgca
aagctgagat gattggtgga gcggggatac ctgcagccct atggaaaggt cattgatctt
ggatgtggca gagggggctg gagttactac gccgccacca tccgcaaagt tcaagaagtg
aaaggataca caaaaggagg ccctgatcat gaagaaccca tgttggtgca aagctatggg
tagaacataa tccgtcttaa gagtgaggtg gacatctttc atatggcgac tgaaccgtgt
gacacgttgc tgtgtgacat aggtgagtca tcatctagtc ctgaagtgga agaagcacgg
acgctcagag tcctttccat ggtgggggat tggcttgaaa aaagaccagg agccttttgt
ataaaagtgt tgtgtccata caccagcact atgatagaaa ccctagagcg actgcagcgt
aggtatgagg gaagactggt cagagtgcca ctctcccaca actctacaca taagatgtac
tgggtctctg gagcgaaaaa caacaccata aaaaatgtgt ccaccacgaa ccagctcctc
ttggggcgca tggacgggcc caggaggcca gtgaaatatg aggaggatgt gaatctcggc
tctggcacgc gggctgtggt aagctgcgct gaagctccca acatgaagat cattggtaac
cacattgaaa agatccacag tgaacacgcg gaaacgtggt tctttgacaa gaaccaccca
tataggacat gggcttacca tgaaagctat aaggccccca cacaaaggtc agcgtcctct
ctaataaacg gggttgtcag gctcctgtca aaaccctggg atgtggtgac tggagtcaca
ggaatagcca tgaccgacac cacaccgtat ggtcagcaaa gagttttcaa ggaaaaagtg
gacactaagg tgccagatcc ccaagaaaac actcgtcagg ttataagcat gatctcttcc
tggttgtgga aagagctaga caaacacaaa cggccacgag tctgtaccaa agaagaattc
atcaacaagg ttcgtagcaa tgcagcatta ggggcaatat ttgaagagga aaaagagtgg
aagactgcag tggaagctgt gaacgatcca aggttctggg ctctagtgga caaggaaaga
gagcaccacc tgagagaaaa gtgccagagt tatatgtaca acatgatgag aaaaaaagaa
aagaaacaag gggaatttgg aaaggccaag agcagccgcg ccatctggta tatgtggcta
ggggctagat ttctagagtt cgaagccctt ggattcttga acgaggatca ctggatgggg
agagagaact caggaggtgg tgttgaaggg ctgggattac aaagactcgg atatgtccta
gaagaaatga gtcgcatacc aagaggaaag atgtatgcag ataacactgc tagctggaac
acccacatca gcaggtttga tctggaaaat gaagctctaa tcaccaacca aatggaaaaa
gggcacaggg ccttggcatt ggccataatc aagtacacat accaaaacaa agtggtaaag
gtccttagac cagctgaaaa agggaagaca gttatggaca ttatttcgag acaagaccaa
aagaggagca aacaagttat cacttacgct cttaacacat ttaccaacct agtagtgcaa
ctcattcgaa gtatgaaggc tgaggaagtt ctagagatac aagacttgtg gctgctgcgg
aggtcagaga aagtgaccaa ctggctgcag agcaacggat gggataggct caaacgaatg
gcagtcagtg gagatgattg cgttgtgagg ccaattgatg ataggtttgc acatgccctc
aggttcttga ataatatggg gaaagttaag aaggacacac aaaaatggaa accctcaact
ggataggaca actgggagga agttccattt tgctcccacc acttcaacaa gctccatctc
aaggacggga ggtccattgt ggttccctgc cgccaccaag atgaactgat tggccgggcc
cgcgtctctc caggggcggg atggagcatc cgggagactg cttgcctagc aaaatcatat
gcgcaaatgt agcagctcct ttatttccac aaaagggacc tccgactgat ggccaatgcc
atttgttcat ctgtgccagt tgactgggtt ccaactggaa gaactacctg gtcaatccat
ggaaagggag aatggatgac cactgaagac atgcttgtgg tgtggaacag agtgtggatt
gaggagaacg accacatgga agacaagacc ccagttacga aatggacaga cattccctat
ttgggaaaaa gggaagactt gtggtgtgga tctctcatag ggcacagacc gcgcaccacc
tgggctgaga acattaaaaa cacagtcaac atggtgcgca ggatcatagg tgatgaagaa
aagtacatgg actacctatc cacccaagtt cgctacttgg gtgaagaagg gtctacacct
ggagtgctgt aa

prM/E proteins include those having at least 80%, 82%, 85%, 87%, 90%, 92%, 95%, 97%, 99% or more amino acid sequence identity to the prM/E proteins encoded by SEQ ID NO:1, SEQ ID NO:2, SEQ ID NO:3, SEQ ID NO:5, SEQ ID NO:11, SEQ ID NO:12, or SEQ ID NO:13.

Capsid proteins include those having at least 80%, 82%, 85%, 87%, 90%, 92%, 95%, 97%, 99% or more amino acid sequence identity to the proteins encoded by one or more of SEQ ID NO:1 SEQ ID NO:2, SEQ ID NO:3, SEQ ID NO:5, SEQ ID NO:11, SEQ ID NO:12, or SEQ ID NO:13.

An exemplary intron/enhancer sequences useful in a vector include: atcgcctggagacgccatccacgctgttttgacctccatagaagacaccgggaccgatccagcctccgcggccgggaa cggtgcattggaacgcggattccccgtgccaagagtgactcaccgtccggatctcagcaagcaggtatgtactctccag ggtgggcctggcttccccagtcaagactccagggatttgagggacgctgtgggctcttctatacatgtaccttttgcttgc ctcaaccctgactatcttccaggtcaggatcccagagtcaggggtctgtattttcctgctggtggctccagttcaggaaca gtaaaccctgctccgaatattgcctctcacatctcgtcaatctccgcgaggactggggaccctgtgacgaac (SEQ ID NO:4), or a nucleotide sequence having at least 80%, 82%, 85%, 87%, 90%, 92%, 95%, 97%, 99% or more nucleotide sequence identity to SEQ ID NO:4.

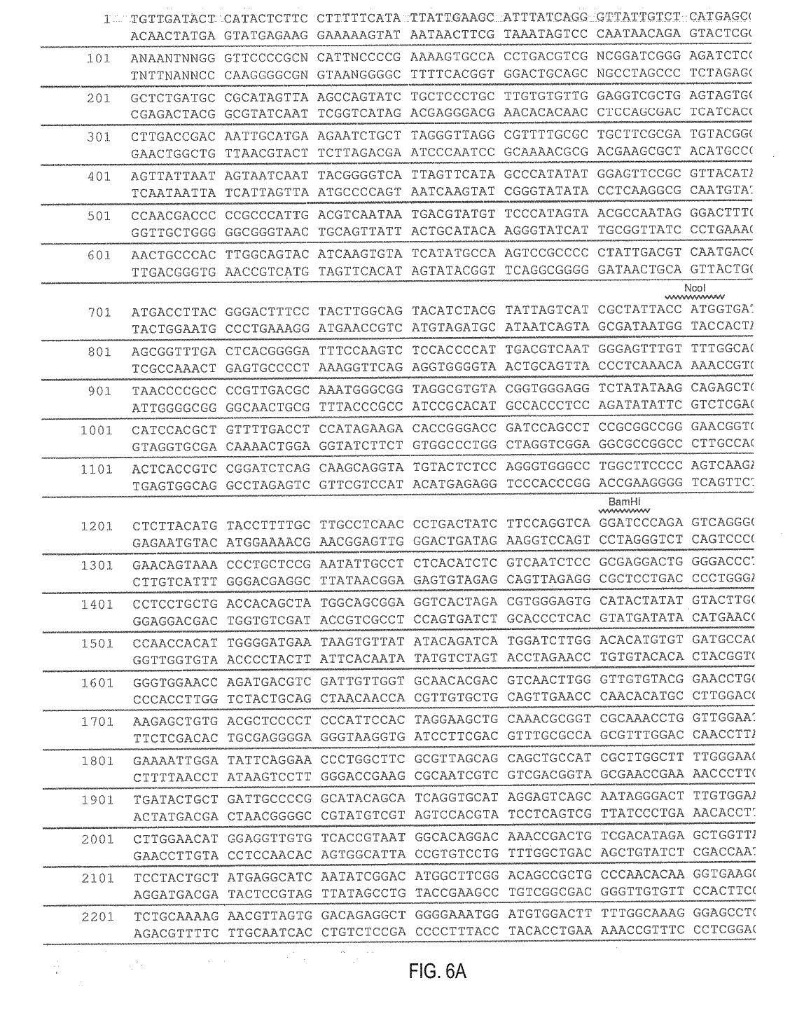

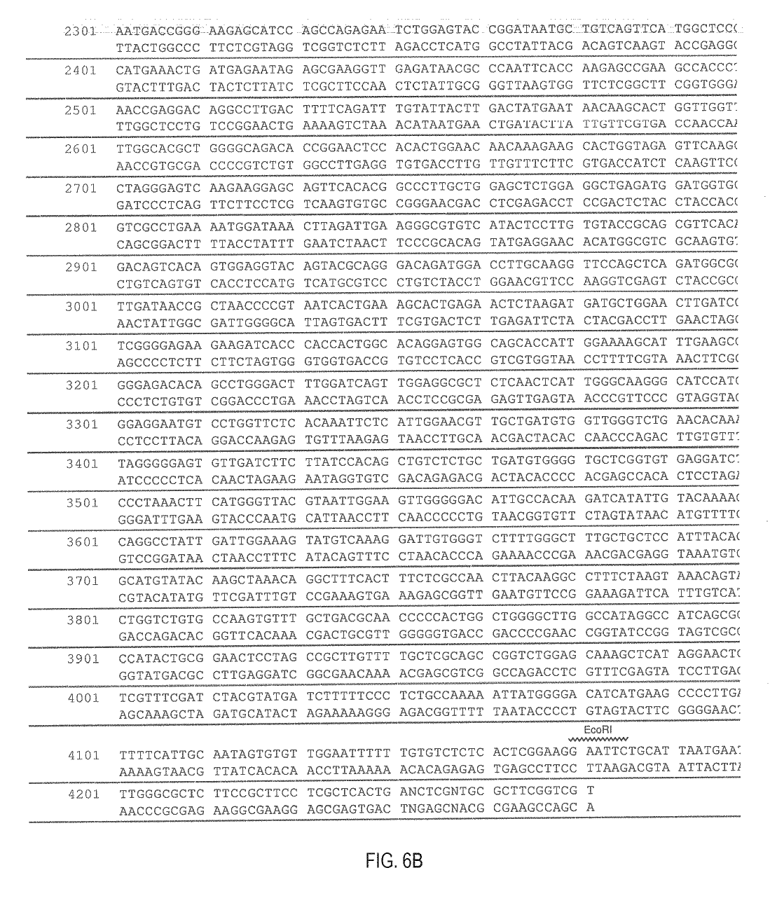

An exemplary vector sequence useful to produce VLPs is shown in FIG. 6 (SEQ ID NO:5).

An exemplary African lineage Zika isolate has the following nucleotide sequence (SEQ ID NO:11 which encodes the protein provided at Accession No. HQ234500 which is incorporated by reference herein):

atgaaaaacc caaagaagaa atccggagga ttccggattg tcaatatgct aaaacgcgga
gtagcccatg taaacccctt gaggggtttg aagaggctgc cggccggact cctgctgggc
catggaccca tcagaatggt tttggcgata ctagccttct tgagattcac agcaatcaag
ccatcactgg gcctcatcaa tagatagggt tccgtgggga agaaggaggc tatggaaata
ataaaaaagt tcaagaaaga tcttgctgcc atgttgagaa taatcaatgc taggaaggag
aggaagagac atggagctaa tgccaacatc ggaatcgtca acctcctgct gactacagtc
atggcagcag agatcactag acgcgggagt gcatactaca tgtacttgga caggagcgat
gctggtaagg ccatttcttt cgttaccaca ctggggatga acaaatgcca tgtgcagatc
atggacctcg ggcatatgtg tgacgccacc atgagttatg agtgccccat gctggacgag
ggagtggagc cagatgacgt cgattgctgg tgcaacacga catcaacttg ggttgtgtac
ggaacctgtc atcataaaaa aggtgaagca cgacaatcca gaagagccgt gacgcttcct
tctcactcta caaggaagtt gcaaacacga tcgcagactt gactagaatc aagagaatac
acaaagcacc tgatcaaggt tgagaattgg atattcagga accccggatt tgcgctagtg
gctgtagcta ttgcctggct cctgggaagc tcgacgagcc aaaaagtcat atacttggtc
atgatattgt tgattgcccc ggcatacagt atcaggtgca taggagttag caataaagac
ttcgtggagg gcatgtcagg tgggacctgg gttgatgttg tcttggaaca tggaggttgt
gtcaccgtga tggcacagga caagccaaca gttgacatag agttggtcac gacaacggtt
agcaacatgg ccgaagtgag atcctactgc tacgaggcat caatatcgga catggcttca
gacagtcact gcccaacaca aagtgaagcc taccttgaca agcaatcaga cactcaatat
gtctataaaa gaacattggt ggacagaggt tgggaaaatg gatgtggact ttttggcaag
gggagcttgg tgacgtgtgc caagtttaca tgctccaaga aaatgacagg gaagagcatc
cagccggaga acttggagta ccggataatg ctatcagtgc atggatccca gcacagtggg
atgattgtga atgacgaaaa cagagcaaaa gtcaaggtta cacccaattc accaaaagca
gaagcaacct tgggaagttt tgaaagcctg agacttgatt gtgaaccaag gacaggcctt
gacttttcag atctgtatta cctgaccatg aacaataacg attggttggt gcacaaagag
tggtttcatg acatcccatt accttggcat tctggtgcag acactgaaac tccacactgg
aacaacaaag aggcactggt gaagttcaag gacgcccacg ccaaaaggca aactgttgta
gttctgggga gccaagaaga agccgttcac acggctctcg ctggagctct ggaggctgag
atggatggtg cgaagggaag gctatcctca ggccatttga aatgccgcct aaaaatggac
aagcttaggt tgaagggtgt gtcatattcc ctgtgtaccg cagcgttcac attcaccaag
gttccagctg aaacattgca tggaacagtc acaatggagg tgcagtatac agggaaggat
agaccctgca aggtcccagc ccagatggcg atggacatac agaccctgac cccagttgga
aggctgataa cggctaaccc tgtgatcact gaaagcactg agaattcaaa gatgatgttg
gagctcgacc caccatttgg ggattcttac attgtcatag gagtcgggga caagaaaatc
acccatcact ggcatcggag tagtagcatc atcggaaagg catttgaagc cactgtgaga
ggcgccaaga gaatggcagt cttgggagac acagcctggg actttggatc agttggaggt
gtgtttaact cattgggcaa gggtattcac cagatctttg gagcagcttt caaatcactg
ttcggaggaa tgtcctgatt ctcacagatc ctcataggca cactgttggt gtggttgggt
ctgaacacaa agaatggatc tatctccctc acatgcttgg ccttgggaag agtgatgatc
ttcctttcca cggctatttc tgctgatgtg aggtgttcag tggacttctc aaaaaaggaa
acgagatgtg gcacgggggt gttcatctac aatgacgttg aagcctggag ggatcgatac
agataccatc ctgactcccc ccgcagattg gcagcagctg ttaagcaggc ttgggaagag
gggatttatg ggatctcctc catttcgaga atggaaaaca tcatatggaa atcagtggaa
ggggagctta atgcgatcct agaggaaaat ggagtccaac taacagttgt agtgggatct
gtaaaaaacc ccatgtggag aggtccacga agattgccag tgcccgtaaa tgagctgccc
catggctgga aagcctgggg gaaatcgtac tttgttaggg cggcaaagac caacaacagt
tttattgtcg acggtgacac actgaaggaa tgtccgctca aacatagaac atggaatagc
ttccttgtag aggatcacgg gtttggggtc ttccacacca gtgtttggct gaaggtcaga
gaggactatt cattagagtg tgacccagcc gtcataggaa cagctgtcaa gggaaaggag
gctgcacaca gtgatctagg ctattggatt gagagtgaaa agaatgacac atggaggctg
aagagggctc atctgattga gatgaagaca tgtgagtggc caaagtctca cacactgtgg
acagatggag tagaagaaaa tgatctaatc atacccaagt ccttagctga tccactcagc
caccacaaca ccagagagga ttatagaact caagtgaaag gaccatggca tagtgaagag
ctcgaaatcc ggtttgagga atgcccaggc accaaggttc atgtggagga gacatgcgga
actagaggac catctttaag atcaaccact gcaagtggaa gggtcataga ggaatggtgc
tatagggaat acacaatgcc tccactatcg ttccgggcaa aagacgactg ctgatatgga
atggagataa ggcccagaaa ggaaccagag agcaacttag tgaggtctat ggtgacagca
ggatcaaccg atcacatgga tcacttctct cttggagtgc ttgtgattct actcatggtg
caggaagatt tgaaaaagag aatgaccaca aagatcataa tgagcacatc aatggcaata
ctggtagcca tgatcttggg aagattctca atgagtgacc tgactaagct tatgatccta
atggatgcca ctttcgcaga aatgaacact ggagaagatg tagctcactt ggcattagta
gcggcattta aagtcagacc agccttgttg gtttccttca tcttcagagc caactggaca
ccccgtgaga gcatgctgct agccctggct tcgtgtctcc tgcagactgc gatttccgct
cttaaaggca agctgatgat cctcgttaat gaatttgctt tggcctagtt ggcaatacga
acaatggccg tgccacgcac tgataacatc actctagcaa ttctgaccgc tctaacacca
ttagccagag gcacactgct tgtggcatgg agagcgggcc tcgccacttg tagagggttc
atgctcattt ccctgaaagg gaaaggtagt gtgaagaaga acctgccatt tgtcatggcc
ttgggattga ccactgtgag gatagtgaac cccattaatg tgataggact actgttacta
acaaagagtg gaaaacggaa ctggccccct agtgaagtgc ttacagctgt cggcctaata
tgtgcactgg ccggagggtt tgccaaggca gacatagaga tggctgggcc catggctgca
gtaggcctgc taattgtcag ttatgtggtc acgggaaaga gtgtggacat gtacattgaa
aaaacaggta atattacatg ggaaaaagac gcgaaagtca ctggaaacag tcctcagctt
aacgtggcac tagataagag tgatgatttc tctttggtag aggagaatgg cccacccatg
agagagatca tactcaaggt ggtcctgatg gccatctgtg gcatgaaccc aatagccata
cccttcgctg caggagcgtg gtatgtgtat gtaaagactg ggaaaaggag cggtgccctc
tgggacgtgc ctactcccaa aaaagtaaaa aagggagaga ctacagatgg aatgtacaga
gttatgactc gcagactgct gggttcaaca caggttggag taggagtcat gcaagaagga
gtcttccata ccatgtggca cgtcacaaaa ggagccgcat tgaggagcgg tgaaggaaga
cttgatccat actgggggga cgtcaagcag gacctggtgt catattgtgg gccgtggaag
ttgaatgcaa cctgggatag actaaatgag gtgcagcttt tggccgtacc ccccgaagag
agggctaaaa acattcagac tctgcctgga atatttaaaa caaagaatgg ggacatcgga
gcagttgctc tagactaccc tgcaggaacc tcaggatctc cgatcctaga caaatgcgga
agagtgatag gactttatgg caatggggtt gtgatcaaga atggaagcta tgttagtgcc
ataacccagg gaaaaaggga gaaggagact ccggttgagt gctttgaacc ctcgatgcta
aggaagaagc aactaacagt cttggatctg catccaggag ccgggaaaac caggagagtt
cttcctgaaa tagtccgtga agccataaag aagagacttc gcacagtgat cttagcacca
accagggttg ttgctgctga gatggaggaa gccctaagag gacttccggt gcgttacatg
acaacagcaa tcaacgtcac ccattctggg acaaaaatca ttgatttgat gtgccatgcc
accttcactt cacgcctact acaaccaatc agagtcccca actacaacct ttatatcatg
gatgaggctc atttcacaga tccttcaagc atagctgcaa gaggatacat atcaacaagg
gttgaaatgg gcgaggcggc tgctatcttc atgactgcta caccaccagg aacccgcgat
gcgtttccag attccaactc accaatcatg gacacagaag tggaagtccc agagagagcc
tggaactcag gctttgacta ggtgacagac cattctggaa aaacaattta gtttgttcca
agtgtgagaa acggaaatga aatcgcagcc tgtctgacaa aagctggaaa gcgggttata
cagctcagca ggaagacttt tgagacagag tttcagaaga caaaaaatca agagtgggac
tttgtcataa caactgacat ttcagagatg ggtgccaact tcaaggctga ccggatcata
gattccagga aatgcctaaa gccagtcata cttaatggtg agagagtcat cctggctggg
cctatgcccg tcacgcacgc cagtgctgct cagaggagag gacgtatagg caggaacccc
aacaaacctg gagatgagta tatgtatgga ggtgggtgtg cagagactga tgaagaccat
gcacactagc ttgaagcaag aatgcttctc gacaacattt acctccagga tagcctcata
gcctcgctct atcgacctga gactgacaag gttgccgcca ttgaaggaga gttcaagcta
aggacagagc aaaggaagac ctttgtagaa ctcatgaaga gaggagacct tcccgtttgg
ctggcctatc aagtagcatc tgccggaata acttacacag acagaagatg gtgctttgat
ggcactacca acaacaccat aatggaagac agtgtaccag cagaggtgtg gaccaagtat
ggaaagaaga aagtgctcaa accgaagtgg atgaatgcca aggtctgttc agatcatgcg
actttgaaat cgttcaaaga atttgccgct aggaagagag gagcgacttt ggaagtaatg
gatgccctag gaacattgcc aggacacatg acagagaggt ttcaggaagc cattgacaat
ctcgctgtgc tcatgcgagc agagactgga agtaggccct acaaagcagc ggcagctcaa
ctgccggaga ccctagagac cattatgctc ttgggtttat tgggaacagt ttcgctagga
atcttctttg tcttgatgca gaacaaaggc atcaggaaga taggcttcga aatggtaacc
cttggggcca gcgcatggct catgtggctt tcggaaattg aaccagccag aatcgcatgt
gtcctcattg tcgtgtttct gttactggtg gtgctcatac ctgagccaga gaagcaaaga
tctccccagg acaatcaaat ggcaatcatc atcatggtgg cagtgggcct tctggatttg
ataactgcaa acgaactcgg atagctggaa agaacaaaaa gtgatatagc tcatctaatg
ggaaggaaag aagaggggac aaccgtagga ttctcaatgg atattgatct gcggccagcc
tccgcctggg ctatttatgc cgcattgaca actctcatca ccccagccgt ccaacatgcg
gtgaccacct catacaacaa ctactccctg atggcgatgg ccacacaagc tagagtgcta
tttgacatgg gcaaagggat gccattttat gcataggact ttggagtccc gctgctaatg
atgggttgtt actcacaatt aacacccctg accctgatag tggccatcat tctgcttgtg
gcacactaca tgtatttgat cccaggtttg caggcagcag cagcacgtgc cgcccagaag
aggacagcag ctggcatcat gaagaatccc gttattgatg aaatagtgat gactgacatt
aacacaataa caattaaccc ccaagtggag aagaagatag gacaaatgtt actcatagca
gtagctgcct ccagtgccgt gctgctgcgg accgcttggg gatgggggga ggctggggct
ctgatcacag cagcaacctc caccttatgg gaaggctctc caaacaaata ctggaactcc
tctacagcca cttcactgtg caatatcttc agaggaaatt atttagcagg gacttccctt
atttacacag taacaagaaa tgccggtctg gttaagagac gtggaggtga aacgggagag
actctgggag agaagtggaa agcccgcctg aaccagatgt cggctttgga gttctattct
tacaaaaagt caggcatcac cgaagtgtgt agggaggagg cacgccgcgc cctcaaggat
ggaatggcca caggaggaca tgctgtatcc cggagaagcg caaagcttag atggttggta
aagagaggat acctgcagcc ccatggaaag attgttgacc tcggatgtgg caaagggggc
tggagttatt acgctgccac catccgtaaa gtgcaggagg tcagaggata cacaaaggga
ggtcctgatc atgaagaacc catgctggtg caaagctatg ggtggaacat agttcgcctc
aagagtggag tggacgtctt tcacatggcg gctgagccgt gtgacacttt gctgtgtgac
attgacgagt catcgtccaa tcctgaagtg gaagagacgc gaacactcaa agtgctctcc
atggtgggag actggctcga gaaaagacca ggggccttct gcataaaggt gctgtgccca
tacaccagta ctatgatgga gaccatggag cgactgcaac gtaggtatgg gggaggattg
gtcagagtgc cattgtcccg caactccaca catgagatgt attgggtctc tggagccaaa
aataacatca taaagaatat gtccaccaca aatcagctcc tcttggaacg catagatggg
cctaggaggc cagtgaaata tgaagaggat gtgaacctcg gctcaggcac acgagctgtg
gcaagctgtg ctgaggctcc caacatgaag atcattggta ggcgcattga gagaatccgc
aatgaacatg caaaaacatg gttctttaat gaaaaccacc catacaggac atgggcctac
catggaaact acaaagcccc cacgcagaag tcagcatcat ccctcgtgaa cagggttatt
agactcctgt caaagcccta ggatgtagtg actgaagtca caggaatagc tatgactgac
accacgccat acggccaaca aagagtcttc aaagaaaagg tggacactag ggtgccagac
ccccaagaag gcacccgccg agtaatgaac atgatctcgt cttggctatg gaaggagctg
gaaaaacgca agcggccacg tgtctacacc aaaaaagagt tcatcaataa ggtacacagc
aatgcagcac taggaacaat atttgaagag aaaaaagaat ggaagacagc tgtagaagct
gtgaatgatc cgagattttg ggctctagtg gacaaggaaa gagaacacca cctgagagga
gagtgtcaca gctgtgtgta caacatgatg ggaaaaagag aaaagaagca aggagaattc
gggaaagcaa aaagcagccg cacaatctag tacatatagt tgagagccag atttctgaaa
tttgaggctc ttggattctt gaatgaagac cattagatgg gaagagaaaa ctcaggaggt
ggcgttgaag ggctaggact gcaaaggctt ggatacattc tagaagaaat gaaccgggcg
ccaggaggaa agatgtatgc agatgacacc gctggctggg atacccgtat tagcaggttt
gatctggaga atgaagccct gatcactaac cagatggaaa aagggcacag agctctggcg
ttggccgtaa ttaaatacac ataccaaaac aaagtggtaa aggttctcag accagctgaa
ggagggaaaa cagtcatgga catcatctca agacaagacc agagagggag cggacaagtt
gttacttatg ctctcaacac attcaccaac ctggtggtgc agcttatccg gaacatggag
gctgaagagg tgctagagat gcatgatcta tggctattga ggaaaccaga gaaagtgacc
agatagttgc agagcaatga ataggacaga ctcaaacgaa tagcagtcaa tggagatgac
tgcgttgtaa agccaattga tgataggttt gcacatgccc tcaggttctt gaatgacatg
ggaaaagtta ggaaagacac acaggaatgg aaaccctcga ctggatggag caattgggaa
gaaatcccgt tctgttccca ccacttcaac aagctgcacc tcaaggatag gagatccatt
atggtcccct gccgccacca agatgaactg attggccgag cccgtatctc accaggggca
ggatggagca tccgagagac tgcctgtctt gcaaaatcat atgcccagat gtggcagctt
ctttatttcc acagaagaga cctccgactg atggccaatg ccatctgttc ggccgtgcca
gccgactagg tcccaactgg gagaaccacc tggtcaatcc atagaaaggg aaaatggata
actaatgagg acatgctcat ggtgtgaaat agagtgtgga ttgaggagaa cgaccacatg
ggggacaaga cccctgtaac aaaatggaca gacattccct atttgggaaa aagggaggac
ttatggtgtg gatcccttat agggcacaga cctcgcacca cttgggctga gaacatcaaa
gacacagtca acatggtgcg tagaatcata gataatgaaa aaaggtacat ggactaccta
tccacccaag tacgctactt ggatgaggag aggtccacac ctggaatgct g

An exemplary Asian lineage Zika isolate has the following sequence (SEQ ID NO:12 which encodes the protein provided at Accession No. HQ234499 which is incorporated by reference herein):

ATGAAAAACC CAAAAAAGAA ATCCGGAGGA TTCCGGATTG
TCAATATGCT AAAACGCGGA GTAGCCCGTG TGAGCCCCTT
TGGGGGCTTG AAGAGGCTAC CAGCTGGACT TCTGCTGGGT
CATGGACCCA TCAGGATGGT CTTGGCGATA CTAGCCTTCT
TGAGATTCAC GGCAATCAAG CCATCACTGG GTCTCATCAA
TAGATGGGGT TCCGTGGGGA AAAAAGAGGC TATGGAAATA
ATAAAGAAGT TCAAGAAAGA TCTGGCTGCC ATGCTGAGAA
TAATCAATGC TAGGAAGGAG AAGAAGAGAC GTGGCGCAGA
CACCAGTGTC GGAATTGTTG GCCTCCTGCT GACCACAGCC
ATGGCAGTGG AGGTCACCAG ACGTGGGAGT GCATACTATA
TGTACTTAGA CAGAAGCGAT GCTGGGGAGG CCATATCTTT
TCCAACCACA CTGGGGGTGA ATAAGTGTTA CATACAGATC
ATGGATCTTG GACACATGTG TGATGCCACA ATGAGCTATG
AATGCCCTAT GTTGGATGAG GGGGTAGAAC CAGATGACGT
CGATTGCTGG TGCAACACGA CATCGACTTG GGTTGTGTAC
GGAACCTGCC ATCACAAAAA AGGTGAGGCA CGGAGATCTA
GAAGAGCTGT GACGCTCCCC TCTCATTCCA CTAGGAAGCT
GCAAACGCGG TCGCAGACCT GGTTGGAATC AAGAGAATAC
ACAAAGCACT TGATCAGAGT CGAAAATTGG ATATTCAGGA
ACCCTGGCTT TGCGTTGGCA GCAGCTGCCA TTGCTTGGCT
TTTGGGAAGC TCAACGAGCC AAAAAGTCAT ATACTTGGTC
ATGATACTGT TGATTGCCCC GGCATACAGT ATCAGGTGCA
TAGGAGTCAG CAATAGGGAT TTTGTGGAAG GTATGTCAGG
TGGGACCTGG GTTGATGTTG TCTTGGAACA TGGAGGTTGT
GTTACCGTAA TGGCACAGGA CAAGCCAACT GTTGATATAG
AGTTGGTCAC AACAACGGTT AGCAACATGG CGGAGGTAAG
ATCCTACTGC TACGAGGCAT CAATATCGGA CATGGCTTCG
GACAGCCGCT GCCCAACACA AGGTGAAGCC TACCTTGACA
AGCAGTCAGA CACTCAATAT GTTTGCAAAA GAACGTTAGT
GGACAGAGGT TGGGGAAATG GATGTGGACT CTTTGGCAAA
GGGAGCCTGG TGACATGCGC CAAGTTTGCA TGCTCCAAGA
AAATGACTGG GAAGAGCATC CAGCCAGAGA ACCTGGAGTA
CCGGATAATG CTGTCAGTTC ATGGCTCCCA GCACAGTGGG
ATGATTGTTA ATGACANAGG ACATGAAACT GATGAGAATA
GAGCGAAGGT TGAGATAACG CCCAATTCAC CAAGAGCCGA
AGCCACCCTG GGAGGTTTTG GAAGCCTAGG ACTTGATTGT
GAACCGAGGA CAGGCCTTGA CTTTTCAGAT TTGTATTACT
TGACTATGAA TAACAAGCAT TGGTTGGTGC ACAAGGAGTG
GTTCCATGAC ATTCCACTAC CTTGGCATGC TGGGGCAGAC
ACCGGAACTC CACATTGGAA CAACAAAGAA GCATTGGTAG
AGTTCAAGGA CGCACATGCC AAAAGGCAAA CTGTCGTGGT
TCTAGGGAGT CAAGAAGGAG CCGTTCACAC GGCTCTTGCT
GGAGCCCTGG AGGCTGAGAT GGATGGTGCA AAGGGAAGGC
TGTCCTCTGG CCACTTGAAA TGTCGCTTGA AAATGGACAA
ACTTAGATTG AAGGGCGTGT CATACTCCTT ATGTACCGCG
GCGTTCACAT TCACCAAGAT CCCGGCTGAA ACGCTGCATG
GGACAGTCAC AGTGGAGGTA CAGTATGCAG GGACAGATGG
ACCCTGCAAG GTTCCAGCTC AGATGGCGGT GGATATGCAA
ACTCTGACCC CAGTTGGGAG GTTGATAACC GCTAACCCTG
TGATCACTGA AAGCACTGAG AATTCAAAGA TGATGTTGGA
ACTTGACCCA CCATTTGGGG ATTCTTACAT TGTCATAGGA
GTTGGGGATA AGAAGATCAC CCACCACTGG NACAGGAGTG
GCAGCACCAT CGGAAAAGCA TTTGAAGCCA CTGTGAGAGG
CGCCAAGAGA ATGGCAGTCT TGGGAGACAC AGCCTGGGAC
TTTGGATCAG TCGGAGGTGC TCTCAACTCA TTGGGCAAGG
GCATCCATCA AATTTTTGGA GCAGCTTTCA AATCATTGTT
TGGAGGAATG TCCTGGTTCT CACAAATCCT CATAGGAACG
TTGCTGGTGT GGTTGGGTCT GAACACAAAG AATGGATCTA
TTTCCCTTAC GTGCTTGGCC TTAGGGGGAG TGTTGATCTT
CCTATCTACA GCCGTCTCTG CTGATGTGGG GTGTTCGGTG
GACTTCTCAA AGAAGGAAAC GAGATGCGGT ACGGGGGTGT
TCGTCTATAA CGACGTTGAA GCCTGGAGGG ACAGGTACAA
GTACCATCCT GACTCCCCTC GTAGATTGGC AGCAGCAGTC
AAGCAGGCCT GGGAAGATGG GATCTGTGGG ATCTCCTCTG
TTTCAAGAAT GGAAAACATT ATGTGGAGAT CAGTAGAAGG
GGAGCTCAAC GCAATTCTGG AAGAGAATGG AGTTCAACTG
ACGGTCGTTG TGGGATCTGT AAAAAACCCC ATGTGGAGAG
GTCCGCAGAG GTTGCCTGTG CCTGTGAATG AGCTGCCCCA
CGGTTGGAAG GCCTGGGGGA AATCGTACTT TGTCAGGGCA
GCAAAGACCA ACAACAGCTT TGTTGTGGAT GGTGACACAC
TGAAGGAATG CCCGCTCAAA CACAGAGCAT GGAACAGCTT
TCTTGTGGAG GATCACGGGT TCGGGGTATT TCACACTAGT
GTCTGGCTTA AAGTCAGAGA GGATTACTCA TTAGAGTGTG
ATCCAGCCGT CATAGGAACA GCTGCTAAGG GAAAGGAGGC
CGTGCACAGT GATCTAGGCT ACTGGATTGA GAGTGAAAAG
AACGACACAT GGAGGCTGAA GAGGGCTCAC CTGATCGAGA
TGAAAACATG TGAATGGCCA AAGTCCCACA CACTGTGGAC
AGATGGAATA GAAGAAAGTG ATCTGATCAT ACCTAAGTCT
TTAGCTGGGC CACTCAGCCA CCACAACACC AGAGAGGGCT
ACAGGACTCA AGTGAAAGGG CCGTGGCATA GTGAAGAGCT
TGAAATCCGG TTTGAGGAAT GTCCAGGCAC CAAGGTCCAC
GTGGAGGAAA CATGTGGAAC GAGAGGACCG TCCCTGAGAT
CAACCACTGC AAGCGGAAGG GTGATCGAGG AATGGTGCTG
CAGGGAATGC ACAATGCCCC CATTGTCGTT CCGGGCAAAA
GATGGCTGTT GGTATGGAAT GGAGATAAGG CCCAGGAAGG
AACCAGAGAG TAACCTAGTA AGGTCAATGG TGACTGCAGG
ATCAACTGAT CACATGGATC ACTTCTCCCT TGGAGTGCTT
GTGATTCTGC TCATGGTGCA GGAAGGGCTG AAGAAGAGAA
TGACCACAAA GATCATCATA AGCACATCAA TGGCAGTGTT
GGTAGCTATG ATCCTGGGAG GATTTTCAAT GAGTGACTTG
GCTAAGCTTG CAATTCTGAT GGGTGCCACC TTCGCGGAAA
TGAACACTGG AGGAGATGTA GCTCATCTGG CGCTGATAGC
GGCATTCAAA GTCAGACCCG CGTTGCTGGT CTCTTTCATC
TTCAGAGCCA ATTGGACACC CCGTGAGAGC ATGCTGCTGG
CCTTGGCCTC GTGCCTTCTG CAAACTGNGA TCTCCGCCCT
GGAAGGCGAC CTGATGGTTC TCATCAATGG TTTTGCTTTG
GCCTGGTTGG CAATACGAGC GATGGCTGTT CCACGCACTG
ACAACATCAC CTTGGCAATC CTGGCTGCTC TGACACCACT
GGCCCGAGGC ACACTGCTTG TAGCGTGGAG AGCAGGCCTT
GCTACTTGTG GGGGGTTCAT GCTCCTCTCT CTGAAGGGGA
AAGGTAGTGT GAAGAAGAAC CTACCATTTG TCATGGCCTT
GGGACTAACC GCTGTGAGGC TGGTTGACCC CATCAACGTG
GTGGGACTGC TGTTGCTCAC AAGGAGTGGG AAGCGGAGCT
GGCCCCCTAG TGAAGTACTC ACAGCTGTTG GCCTGATATG
TGCACTGGCC GGAGGGTTCG CCAAAGCAGA TATAGAGATG
GCTGGGCCCA TGGCTGCAGT TGGCCTGCTA ATTGTTAGTT
ACGTGGTCTC AGGAAAGAGT GTGGACATGT ACATTGAAAG
AGCAGGTGAC ATCACATGGG AAAAAGATGC GGAAGTTACT
GGAAACAGCC CCCGGCTCGA TGTGGCACTA GATGAGAGTG
GTGATTTCTC CCTGGTGGAG GATGATGGTC CCCCCATGAG
AGAGATCATA CTCAAGGTGG TCCTGATGAC CATCTGTGGC
ATGAACCCAA TAGCCATACC CTTTGCAGCT GGAGCGTGGT
ATGTGTATGT GAAGACTGGA AAGAGGAGTG GTGCTCTATG
GGATGTGCCT GCTCCCAAGG AAGTAAAAAA GGGGGAGACC
ACAGATGGAG TGTATAGAGT GATGACTCGC AGACTGCTAG
GTTCAACACA AGTTGGAGTG GGAGTCATGC AAGAGGGGGT
CTTCCACACT ATGTGGCACG TCACAAAAGG ATCCGCGCTG
AGGAGCGGTG AAGGGAGACT TGATCCATAC TGGGGAGATG
TTAAGCAGGA TCTGGTGTCA TACTGTGGCC CGTGGAAGCT
AGATGCCGCT TGGGACGGAC ACAGCGAGGT GCAGCTTTTG
GCCGTGCCCC CCGGAGAGAG AGCGAGGAAC ATCCAGACTC
TGCCCGGAAT ATTCAAGACA AAGGATGGGG ACATCGGAGC
AGTTGCTCTG GACTACCCAG CAGGAACTTC AGGATCTCCG
ATCCTAGACA AGTGTGGGAG AGTGATAGGA CTCTATGGCA
ATGGGGTCGT GATCAAAAAT GGAAGTTATG TTAGTGCCAT
CACCCAAGGG AGGAGGGAGG AAGAGACTCC TGTTGAATGC
TTCGAACCTT CGATGCTGAA GAAGAAGCAG CTAACTGTCT
TGGATCTGCA TCCTGGAGCT GGGAAAACCA GGAGAGTTCT
TCCTGAAATA GTCCGTGAAG CCATAAAAAC AAGACTCCGC
ACGGTGATCC TGGCTCCAAC CAGGGTTGTC GCTGCTGAAA
TGGAGGAAGC CCTTAGAGGG CTTCCAGTGC GTTACATGAC
AACAGCAGTT AATGTCACCC ACTCTGGGAC AGAAATCGTT
GATTTAATGT GCCATGCCAC CTTCACTTCA CGCCTACTAC
AACCCATTAG AGTCCCCAAC TACAATCTTT ACATTATGGA
TGAGGCCCAC TTCACAGATC CCTCAAGTAT AGCAGCAAGA
GGATACATAT CAACAAGGGT TGAGATGGGC GAGGCGGCTG
CCATCTTCAT GACCGCCACA CCACCAGGAA CCCGCGACGC
ATTTCCGGAC TCTAACTCAC CAATCATGGA CACAGAAGTG
GAAGTCCCAG AGAGAGCCTG GAGCTCAGGC TTTGATTGGG
TGACGGATCA TTCTGGAAAA ACAGTTTGGT TTGTTCCAAG
CGTGAGGAAC GGCAACGAGA TCGCGGCTTG TCTGACAAAA
GCTGGAAAAC GGGTCATACA GCTCAGCAGA AAGACTTTTG
AGACAGAGTT CCAGAAAACA AAAAATCAAG AGTGGGACTT
CGTCGTAACA ACTGACATCT CAGAGATGGG CGCCAACTTC
AAAGCTGACC GGGTCATAGA TTCCAGGAGA TGCCTGAAGC
CGGTCATACT TGATGGCGAG AGAGTCATTC TGGCTGGACC
CATGCCTGTC ACACATGCCA GCGCTGCCCA GAGGAGGGGG
CGCATAGGCA GGAATCCCAA CAAACCTGGA GATGAGTATA
TGTATGGAGG TGGGTGCGCA GAGACTGATG AAGACCATGC
ACACTGGCTT GAAGCAAGAA TGCTTCTTGA TAACATTTAC
CTCCAAGATG GCCTCATAGC CTCGCTCTAT CGACCTGAGG
CCGATAAGGT AGCAGCCATT GAGGGAGAGT TCAAGCTTAG
GACGGAGCAA AGGAAGACCT TTGTGGAACT CATGAAAAGA
GGAGATCTTC CTGTTTGGCT GGCCTATCAG GTTGCATCTG
CCGGAATAAC CTACACAGAT AGAAGATGGT GTTTTGATGG
CACGACCAAC AACACCATAA TGGAAGACAG TGTGCCGGCA
GAGGTGTGGA CCAGATACGG AGAGAAAAGA GTGCTCAAAC
CGAGGTGGAT GGACGCCAGA GTTTGTTCAG ATCATGCGGC
CCTGAAGTCA TTCAAAGAAT TTGCCGCTGG GAAAAGAGGA
GCGGCCTTTG GAGTGATGGA AGCCCTGGGA ACACTGCCAG
GACACATGAC AGAGAGGTTT CAGGAAGCCA TTGACAACCT
CGCTGTGCTC ATGCGGGCAG AGACTGGAAG CAGGCCCTAC
AAAGCCGCGG CGGCCCAATT ACCGGAGACC TTAGAGACCA
TCATGCTTTT GGGTTTGCTG GGAACAGTCT CGCTGGGAAT
CTTCTTTGTC TTGATGCGGA ACAAGGGCAT AGGGAAGATG
GGCTTTGGAA TGGTGACCCT TGGGGCCAGT GCATGGCTTA
TGTGGCTCTC GGAAATTGAG CCAGCCAGAA TTGCATGTGT
CCTCATTGTC GTGTTTCTAT TGCTGGTGGT GCTCATACCT
GAGCCAGAAA AGCAGAGATC TCCCCAGGAC AACCAAATGG
CAATTATCAT CATGGTAGCA GTGGGTCTTC TGGGCTTGAT
AACCGCCAAT GAACTCGGAT GGTTGGAGAG AACAAAAAGT
GACCTAGGCC ATCTAATGGG AAGGAGAGAG GAGGGGGCAA
CCATGGGATT CTCAATGGAC ATTGACTTGC GGCCAGCCTC
AGCTTGGGCT ATCTATGCCG CTCTGACAAC TCTCATCACC
CCAGCCGTCC AACATGCGGT AACCACTTCA TACAACAACT
ACTCCTTAAT GGCGATGGCC ACGCAAGCCG GAGTGTTGTT
TGGCATGGGC AAAGGGATGC CATTCTATGC GTGGGACTTC
GGAGTCCCGC TGCTAATGAT GGGTTGCTAC TCACAATTAA
CACCCTTGAC CTTAATAGTG GCCATCATTC TGCTCGTGGC
GCACTACATG TACTTGATCC CAGGTCTACA GGCAGCAGCG
GCGCGCGCTG CCCAGAAGAG AACGGCAGCT GGCATCATGA
AGAACCCTGT TGTGGATGGA ATAGTGGTGA CTGACATTGA
CACAATGACA ATTGACCCCC AAGTGGAGAA AAAGATGGGA
CAAGTGCTAC TCATAGCAGT AGCCATCTCC AGTGCCGTTC
TGCTGCGCAC CGCCTGGGGG TGGGGGGAGG CTGGGGCCCT
GATCACAGCC GCAACTTCCA CTTTGTGGGA AGGCTCTCCG
AATAAATACT GGAACTCCTC CACAGCCACT TCACTGTGTA
ACATTTTTAG GGGAAGTTAC TTGGCTGGAG CTTCTCTTAT
TTACACAGTA ACAAGAAACG CTGGCCTGGT CAAGAGACGT
GGAGGTGGAA CGGGAGAGAC CCTGGGGGAG AAATGGAAGG
CCCGCCTGAA CCAGATGTCG GCCCTGGAGT TTTACTCCTA
CAAAAAGTCA GGCATCACCG AAGTGTGCAG AGAAGAAGCC
CGCCGCGCCC TCAAGGACGG AGTGGCAACA GGAGGCCATG
CTGTGTCCCG AGGAAGCGCA AAGCTTAGAT GGTTGGTGGA
GAGAGGATAC CTGCAGCCCT ATGGAAAGGT CATTGATCTT
GGATGTGGCA GAGGGGGCTG GAGTTACTAC GCCGCCACCA
TCCGCAAAGT TCAAGAGGTG AAAGGATACA CAAAGGGAGG
CCCTGGTCAT GAAGAACCCA CGTTGGTGCA AAGCTATGGA
TGGAACATAG TCCGTCTTAA GAGTGGGGTG GACGTCTTTC
ACATGGCGGC GGAGTCGTGT GACACTTTGC TGTGTGACAT
AGGTGAGTCA TCATCTAGTC CTGAAGTGGA AGAAGCACGG
ACGCTCAGAG TACTCTCCAT GGTGGGGGAT TGGCTTGAAA
AAAGACCAGG GGCCTTTTGT ATAAAGGTGT TGTGCCCATA
CACCAGCACC ATGATGGAAA CCCTAGAGCG ACTGCAGCGT
AGGTATGGGG GAGGACTGGT CAGAGTGCCA CTCTCCCGCA
ACTCTACACA TGAGATGTAC TGGGTCTCTG GAGCGAAAAG
CAACATCATA AAAAGTGTGT CCACCACGAG CCAGCTCCTC
TTGGGACGCA TGGACGGGCC CAGGAGGCCA GTGAAATATG
AGGAGGATGT GAATCTCGGC TCCGGCACGC GAGCTGTGGC
AAGCTGCGCC GAAGCTCCCA ACCTGAAGAT CATTGGTAAC
CGCGTTGAGA GGATCCGCAG TGAGCATGCG GAAACGTGGT
TCTTTGATGA GAACCACCCA TACAGGACAT GGGCTTACCA
TGGGAGCTAC GAGGCCCCTA CACAAGGGTC AGCGTCTTCT
CTCATAAACG GGGTTGTCAG GCTCCTGTCA AAGCCCTGGG
ATGTGGTGAC TGGAGTCACA GGAATAGCCA TGACCGACAC
CACACCGTAT GGCCAGCAAA GAGTTTTCAA GGAAAAAGTG
GACACTAGGG TGCCAGACCC CCAGGAAGGC ACTCGTCAGG
TGATGAACAT GGTCTCTTCC TGGCTATGGA AGGAGCTAGG
TAAACACAAA CGGCCACGAG TTTGCACCAA AGAAGAGTTC
ATCAATAAGG TTCGCAGCAA TGCAGCACTG GGGGCAATAT
TTGAAGAGGA GAAAGAATGG AAGACTGCAG TGGAAGCTGT
GAACGATCCA AGGTTCTGGG CCCTAGTGGA CAAGGAAAGA
GAGCACCACT TGAGAGGAGA GTGTCAGAGC TGTGTGTACA
ACATGATGGG AAAAAGAGAA AAGAAGCAAG GGGAATTTGG
AAAGGCCAAG GGCAGCCGCG CCATTTGGTA CATGTGGCTA
GGGGCTAGAT TTCTAGAGTT TGAAGCCCTT GGATTCTTGA
ACGAGGATCA CTGGATGGGG AGAGAGAATT CAGGAGGTGG
TGTTGAAGGG CTGGGATTAC AAAGACTTGG ATATGTTCTA
GAAGAAATGA GCCGCACACC AGGAGGAAAG ATGTATGCAG
ATGATACCGC TGGCTGGGAC ACCCGCATCA GTAGGTTTGA
TCTGGAGAAT GAAGCTCTGA TCACCAACCA AATGGAGAAA
GGGCACAGGG CCTTGGCGTT GGCCATAATC AAGTACACAT
ACCAAAACAA AGTGGTAAAG GTCCTTAGAC CAGCTGAAAG
AGGGAAGACA GTTATGGACA TCATCTCAAG ACAAGACCAA
AGAGGGAGCG GACAAGTTGT TACTTACGCT CTTAATACAT
TCACCAACCT GGTGGTGCAG CTCATTCGGA ACATGGAGGC
TGAGGAAGTT CTAGAGATGC AAGACTTGTG GCTGTTGAGG
AGGCCAGAGA AGGTGACCAG CTGGTTGCAG AGCAACGGAT
GGGATAGGCT CAAACGAATG GCAGTCAGTG GAGATGATTG
TGTTGTGAAA CCAATTGATG ATAGGTTTGC ACATGCCCTC
AGGTTTTTGA ATGACATGGG GAAAGTTAGG AAGGACACAC
AGGAGTGGAA ACCCTCAACT GGATGGAGCA ACTGGGAAGA
AGTTCCGTTT TGCTCCCATC ACTTCAACAA GCTTTACCTC
AAGGACGGGA GGTCCATTGT GGTCCCCTGT CGCCACCAAG
ATGAACTGAT TGGCCGAGCC CGCGTCTCAC CAGGGGCGGG
ATGGAGCATC CGGGAGACTG CTTGCCTAGC AAAATCATAT
GCACAAATGT GGCAGCTTCT TTATTTCCAC AGAAGGGACC
TCCGACTGAT GGCCAACGCC ATTTGTTCAT CTGTGCCAGT
TGACTGGGTT CCAACTGGGA GAACCACCTG GTCAATCCAT
GGAAAGGGAG AATGGATGAC CACTGAGGAC ATGCTTGTGG
TGTGGAACAG AGTGTGGATT GAGGAGAACG ACCACATGGA
GGACAAGACC CCAGTCACGA AATGGACAGA CATTCCCTAT
TTGGGAAAAA GGGAAGACTT ATGGTGTGGA TCTCTTATAG
GGCACAGACC ACGCACTACT TGGGCTGAGA ACATTAAAGA
CACAGTCAAC ATGGTGCGCA GGATCATAGG TGATGAAGAA
AAGTACATGG ACTACCTATC CACTCAAGTT CGCTACTTGG
GTGAAGAAGG GTCCACACCT GGAGTGTTA

An exemplary Spodweni virus lineage has the following nucleotide sequence (SEQ ID NO:13 which encodes the protein provided at Accession No. DQ859064, which is incorporated by reference herein:

atgaaaaacc caaaaagagc cggtaggagc cggcttgtca atatgctaaa acgcggtgca
gcccatgtca tccctccaga aggaggactc aagaagctgc ctgtaggatt gctattaggt
cggggtccga tcaaaatgat cctggccata ctggcattcc tacgatttac aacaataaaa
ccgtccactg gcctcatcaa cagatgggga aaagtgggca aaaaagaggc catcaaaatc
ctcacaaaat tcaaggctga cgtgggcacc atgctgcgta tcatcaacaa tcggaagaca
aaaaagagag gagtcaaaac tgaaattgtg ttcctggcat tgctgatgtc tattgttgct
atggaagtca caaaaaaggg ggacacctat tacatgtttg cggacaagaa ggacgccgga
aagatggtga cctttgagac tgaatctgga cccaaccgtt actccatcca agcaatggac
attggacata tgtgtccagc tacaatgagc tatgaatgtc ccgtgctgga accacagtat
gagccagagg atgtcgactg ttggtgcaac tcgacaggag catggattgt gtatggcaca
tgcacccaca aaacaacgga agagacaaga cgttccagac gttcaatcac cctgccatct
catgcctcac aaaaattgga gaccagatca tcgacatagc ttaaatcgcg caaatactcc
aaatatctaa taaaagtgga aaactggatc ctccgcaatc caagatatgc gttggtgact
gcagtgattg gatggactct gggcaggagt cgcagccaga agatcatctt tgtcactctg
ctcatgttgg tagcccccgc atacagcatc agatgcattg gaattggaaa cagagacttc
attgagggaa tgtccagtgg cacctgggtg aacattgtcc tggaacatgg tgattgtgtg
acaataatgt caaacgacaa acccacattg gactttgaac tggtgacaac gaccgcaagt
aacatggcta aggtcaagtc ctactactat gaaactaaca tatccgagat ggcatcggac
aggaggtgcc ccacacaggg ggaagcttat cttgacaaaa tggccgactc ccagtttgtg
tgcaagcgtg ggtacgttga caggggctgg ggaaacggat gtggactctt tggaaaagga
agcattgtca cttgcgctaa gttcacatgt gtgaaaaagc tcacagggaa aagcattcaa
ccggaaaatc tcaaataccg gatcgttatt tcggtacacg cttcccaaca tagaggaata
attaacaatg acaccaatca ccaagacaac aaggaaaaca gaacacgcat taatatcaca
gctagcgctc cccgtgttga ggtggaactt ggctcctttg gatccttctc gatggagtgt
gaaccccggt caggattgaa ctttggtgac ctgtattacc tcaccatgaa caacaagcat
tggctggtta atagaaattg gtttcacgat ctttccttac catggcatac agaagccaca
tcaaacaatc atcactagaa caacaaggag gcgctggtaa aattcaaaaa agcccacgca
aagaagcaga cggctgtgat cctagaaagt cagaaaggaa ctgttcacac agcactggcc
ggcgcactgg aggctgagtc tgatggacac aaagcgacta tctactctgg acacttgaag
tatcgcttga agctagacaa actgcgcctg aagggaatgt catatgcact ctgcacagga
gcattcacct tcgctcgcac cccctctgaa acaattcacg gcaccgccac agtggaactg
caatatgcag gtaaagatgg gccgtgcaaa gttcccatag taattaccag taacaccaat
aggatagcct cgacaggcag gctgatcaca gcgaatccgg tgatcacgga aagtggaaca
aactcaaaga tgatggtcga gattgaccct ccgtttggtg attcttacat tattgtgggc
actggcacaa caaaaattac ccaccattgg cacagagccg gtagttcaat tggacgtgca
tttgaggcta ccatgagagg agcaaaacgg atggcggtcc tcggcaacac cgcttgagac
tttagctcta ttggggacat gttcaactcc gttagaaagt ttgtccacca ggtatttgga
tcaacattta aggcattgtt tggagacatg tcctggttca cacagctect gatagaattt
ctgctcatat ggatgggttt gaacgcacgc ggtggaaccg tggccatgag cttcatgggc
attggggcta tgctgatttt cctagccacc tcggtgtcag gagacacagg atgctcggtt
gacatatcca gaagggaaat gcggtgcggg agcgacatat tcgtgtacaa tgacgttgac
gcatgacaaa gccgctacaa ataccatcct gaaaccccca gaactttggc cactgccata
aaaacagctt ggaaagaagg gacctgtaac attacctcag tgagcagaat gaaaaaccta
atgtggagct ctgtggctgg agagttgaat gcaatccttg aggacaattc agtgccattg
acagtcgtcg ttggcgagcc aaaatatcca ctgtacaatg ctccaaagag gctgaaacca
ccagcatcag agttaccgca ggggtggaag tcctgaggaa agtcatactt tgtctcagcc
gcaaaaaaca acaactcctt tgtagtagat gataacacca tgaaggaatg cccaaaacag
aagcgagcat agaacaactt gagaatagag gatcatgggt tcggagtctt ccacactagc
atctggctga aattccatga ggacaactcc accgaatgtg acacagctat cataggaacg
gcggttcgcg ggaaggaagc cgttcatagt gacttgggct actggataga gagtgagcgc
aatgacacat ggaggctctc tcgagcgcac ctgatcgaag caaagacatg tgaatggcca
cggtcgcaca cactgtggac ggacggagtg aaagagagcg agctgatcat tccacgtggc
ttaaccggtc ctttcaacca tcataacacg catactggct acaagactca gaataaaggt
ccctggcatt taggtgatgt tgaaattcag ttcgccacgt gccccggaac aaccgtggtc
caggaccaag agtgcaggga caggggcgct tctctacgca cgaccacagc tagtggaagg
gtaatcaatg aatggtgcta caggtcatgc accatgcctc cactcagttt caagacaaaa
gatgaatgtt gatatgcaat ggagatacgt cctgtgaaag aacaagagtc aaacctcgtg
cgatcacacg tcactgccgg aagcacaaac cacatagacc atttctctct cagattaata
gtggtcatgt tgatggtgca agaaggtatg aagaagagaa tgacatcaaa agcaataatc
acctcagcgg cctttctcct ggcggttatg atagtgggag gtttcacgta ccaggatttt
aggaggctag tggtattggt ggatgctgca tttgctgaaa tgaacactgg agatgacgtt
acgcacctag cgctgatggc agcgtttaaa atgaggccag cgatgctggt ctcattcatg
ttcagagcct tgtggacccc cagagagtca ctgcttttaa ctctggctac ctgcctcctg
caggtgtcag tgacaccact ggatcattcc atcatgatcg tggttgatgg gattgcgctg
tcctggttgt gtctgaaagc catcttggtg ccgcgtaccc caaacatagc ccttcctctt
ctcgctatgc tgtcacccat gctccaaggt accaccattg tggcatggcg agctatgatg
gcggccctgg ctgtcataac cttggcttcc atgaagcatg gaaggggtgt aaaaaaaacg
tttccctaca ccatcggatg catccttaac agcatagact taattgaaaa cttggggtta
gttggcctcc tcttgttgac agcctcaaaa aagaggagtt ggcctccgag tgaggtgatg
acggctgtcg gactgatctg tgcaattgtg ggcggactaa ccaagaccga cattgacatg
acgggaccca tggcaaccat agaactgctg atggtgagct atgtgatttc tgacaagagt
atggacatat acattaaaaa ggtgtgtgac atatcatgag acaagaacgc tgaaataaca
gacacaagtc cgcggctgaa tgtagctctc gacaacagta aagatttctc acttatccag
gatgacgggc cccccactcg agagattgtg ttgaaggtgt ttctgatgtg tgtttgcggt
gtcagcccca tagccatccc ctttgcagcc gctgcttggt tcgtgtacat taaatcaggg
aaaaaaagcg gcgccatgta ggacattcca tccccaagag aagtgaaaaa aggggaaaca
acggctggag tatacagaat catgacacgt aaattgctgg gcagcacaca ggtgggagcc
ggagtaatgc ataaaggtgt ttttcacaca atgtgacacg tcacaaaagg ttcggccctt
cggagtggtg agggacgcct agatccatac tggggaaacg tgaagcagga tttgatctct
tactgcggac catggaaact ggatgggaaa tgggacggcg tgtcggaagt ccaactgata
acggtcgccc caggtaagcg cgccagaaat atgcagacaa aaccaagagt gttcaagacc
actgatggag aaatcagggc cttggccctt aacttcccag gcggaagttc agactccccg
ataattgaca aaaatgaaca tgtaattggc ctgtatggaa atggtgtcat ggtcaagagt
ggaagctacg tgagtgccat catgcagaca gagaagatgg aggaacccgc agttgactgc
tttgaggagg acatgctgag aaaaaagaag ctgacggtgc tcgacctcca tccaggagct
ggaaaaactc gaagagtgct ccctcaaatc gtcaaggctg caattaagaa acgcctacgc
acggtaatcc tagcacccac ccgagtagtg gcagctgaga tagctgaggc actaaaagac
cttccaataa ggtacatgac tccggcaatt tcagccaccc ataatggcaa taagattatt
gaccttatgt gccacgccac ttttacatca aggctaatgc aaccaattag ggtgcctaat
tacaatctat atataatgga tgaggcccac ttcacagatc ctgcaagcat cgctgcaaga
aggtacatag caacaagagt ggacatggga aacgccgcag ccatcttcat gacggccacc
cctcctggca gcactaaagc tttcccggat tcaaacgccc ccatcacaga tgttgaaaca
gagattccta acaaggcgtg gaattctgga tttaaatgga tcactgatta cccagagaaa
accgtttggt ttgtccctag tgtcagaatg ggcaatgaga tctcggcctg cctcacaaaa
gccggcaaat cggttatcca actcagccgg aaaacctttg aaacagagta ccagaagaca
aagaatggtg aatgggactt tgtcgtaacc actgacatct cagaaatgga agccaacttc
aaggccgaca gagtcataga ctcacgaaaa tgcttgaagc cagtgattct ggatgacatg
gaagaaaaag ttattcttgc caggccgatg gcagtaacac catccagcgc aactcaacgc
agaggaagaa ttggaagaaa ccccaacaaa actggagatg agttctatta cggggggggc
tgtgccgcaa cggatgatga ccatgctcat tgggtagagg ctaggatgct gcttgacaac
atctacctcc aggacaacct cgttgcatct ctgtacaaac cagaacaagg aaaggtctcg
acaatagaag gggagttcaa actgagagga aaacagagaa aaaccttcgt ggagctgatg
aagagaggga acttgccaat gtgattgtca tatcaagtga cggcctccag actcaactat
actgaccggc gctggtgctt tgatggaaaa aacaacaaca ccatcctgga ggactgcgtc
cccgtcgagg tgtggacaaa atttggagag aaaaagattc tgaagcccag atggatggac
gctcagatct gctctgatca tgcctctttg aagtctttca aagagtttgc tgcaggaaag
agaacaatag ccactggctt aattgaagct tttgagatgc ttcccgggca catgactgag
agattccagg aggccgtcga caatttggcc gtgttgatga gggccgaggc aggctctagg
acacacagaa tggctacagc acagctccct aagacaatag aaaccatcct gctcctcagc
ctgctggcat tcgtgtcact tggtgtattt tttatactga tgagggcaaa aggattagga
aaaatggggt ccggcatgat cgtgctggca ggaagtggct ggctcatgtg gatgtctgag
gtggaaccag cccgcatagc ttgtgtggtg atcatagtgt ttctgctaat ggtcgttctg
attccggaac cagagaagca gcgctctccc caggacaatc aactggctct aattatcttg
atcgcgacgg gcctcatcac gctcatcgcg gccaatgagc taggttggtt agaaagaaca
aagagtgacc tcaccaggct gttttggaaa gaacacgctg agccaacagg aaggagaaga
ttttccttct cgctggacat tgacctgcgg ccggcatcgg cctgggcaat atatgccgct
atgacaaccc tgatcacacc gacagtccaa cacgctgtga ccacatcgta caacaactac
tctctcatag ctatgaccac tcaggccgga attctttttg gcatgagacg ggaggtgcct
ttttacaaat gggactttgg cgtgccactc cttatgctag gctgctactc acaacttacc
ccactcaccc tgatcgtgac tctcgtgatg ctaaccgctc actatctcta tctcatcccc
gggctccagg caacggccgc cagggccgcc caacgaagga cggctgctgg aataatgaaa
aacccagtgg tggatggaat tgtggtaact gacatagacc caatccaaat cgatccaaat
gtcgaaaaga agatgggcca ggtcatgctc atctttgtgg ctttggcgag cgcgattctc
atgaaaacgg catggggtta gggagaagct ggtgcccttg catcggcagc agctgccacc
ctatgagaag ggactcccaa caagtactag aattcatcaa cgactacatc cttgtgcaac
atatttcggg gaagttatct ggcaggtccc tccctcatct acaccgtcac acgcaatgca
ggtatcatga agaaaagggg cggtggaaat ggagaaacgg tgggcgagaa atggaaggag
cgcttgaatc ggatgaccgc gcttgaattc tacgcctaca agcggtcagg aataactgaa
atgtgcagag aacccaccag aaaagccttg aaggatggag tcgtcacagg agaacacgct
gtctcccgca aaagcgcaaa gctacaatgg atgatggaac atggccacat caatctagtg
ggacgcgttg tcgacctcgg atgtggaagg ggtggctgga gttactacgc cgcatctcaa
aagcaagtcc tcgaggtgag aggctacaca aaagggggag cgggccacga ggagcccatg
aatgtccaaa gttatggtta gaacatagtg cgactcaaga gtggagtgga cgttttttat
ctaccatcag aaccatgtga cacgctactc tgtgacattg gagagtcatc ctcgaaccca
gcagtagaag aaacccggac tctgagaatg ctcggaatgg ttaaaacctg gctggaacga
ggcgtaaaga acttctgcat caaagtgctc tgcccgtaca ccagtgccat gattgagcgg
ctggaagccc tccagcgtcg ctacggagga ggcctggtga gggttccact ctccagaaat
tccacccacg aaatgtactg ggtctctgga acaaaatcaa acatcatcag gaatgtgaat
accaccagcc agctgctcat gcacagaatg aacatcccca cgcggaaaac aaagtttgaa
gaaaacgtca atctggagac cggaaccagg gcaattgaaa acagagctaa ccctcccgac
atgaaaaaac taggcagccg gattgagcgg ttgagaaagg aatatggatc cacttggcac
tacgatgaaa accaccccta caggacatgg cattaccacg gcagttatga ggctgacacg
caagactccg cctcctcaat ggtcaacggc gtggtgcgtc tcctctcaaa accatgagat
gcattgagct cagtcaccaa cattgctatg acggacacaa ctccgtttga acagcaacgg
gtgttcaagg agaaagtgga cacccggact ccagacccca agcaaggcac gcaaagaatc
atggccataa catcacaatg gctgtgggac cgcctagcaa gaaacaagac ccctcggatg
tgcacgcgac aggaattcat aaacaaggtc aacagtcacg cggcgttggg acccgttttt
agagaacaac agggatgggg ttcagcggcc aaageggtag tagatcctag gttttgggag
ctcgttgaca atgaaagaga agcccatttg agaggggaat gcttgacctg tgtctacaac
atgatgggga aaagagaaaa gaaactcggt gaattcggga aggcaaaaag cagcaaagcc
atttggtaca tgtggctggg agcccgcttc ctcgagttcg aggccctggg cttcctcaat
gaagaccact ggttaagcag agagaactct ggagggggag ttgagggctt gggcctccaa
aaacttggat acatccttga agagatcagc aggaagccag gaggcaaaat gtatgccgat
gacacggctg gctgggacac ccgcatcacg aaatacgacc tagaaaatga ggcgcgcatt
ttggaaaaaa tgaacgggat ccacaaaaaa ctcgcacagg ccatcatcga gttgacatac
aagcataagg ttgtgagagt cttgagacca gcaccacaag ggaaggtcgt tatggacatc
atctccaggc cagaccaaag ggggagtggg caggtggtta cttatgccct caacacctat
acaaacttag tggtgcagct gatccgtaac atggaagcag aggctatcat caatgaaaga
aacatggaag agctccaaaa cccatggaaa atcatcaatt ggctaaaagg aaatggatgg
gacagactcc actcgatgac agtaaatgga gataactgta tcgtgaaacc aatagatgat
aggttcgcct atgcactgaa tttcctcaat gacatgggca aggtcagaaa agatgtccag
gaatggaagc cctcgccggg gtggacaaac tgggaagaag tgcccttttg ctcccaccac
ttcaacaagc tcccgatgaa ggatggaaga acaataatag ttccctgcca gcaccaagat
gagttgatag gcagggctaa agtttctcca ggaaaaggct gatcactcaa tgaaacagca
tgcttgggca agtcttatgc ccagatgtgg ctactgttgt actttcacag gagagatctc
cgactcatgg caaacgcaat ctgctctgct gtaccggtga gttgggtgcc cacggggaga
acaacctggt ccatccatag gcgtgaagag tggatgacaa cagaggacat gctagaggta
tggaacagag tgtggatcat agagaatgag tacatggagg acaagacccc tgtcacagag
tggaccgatg ttccatactt gggaaagaga gaagacttgt ggtgcggctc ccttattgga
cacaggccaa gaagcacatg ggcagagaac atctgggctg ccatttatca agtgcgccga
gcaatcggcg aaactgaaga atatagagac tacatgagca cacaggtccg ctatggctcg
gaggaagagc caagcgctgg tatgttgtaa

### EXAMPLE 3

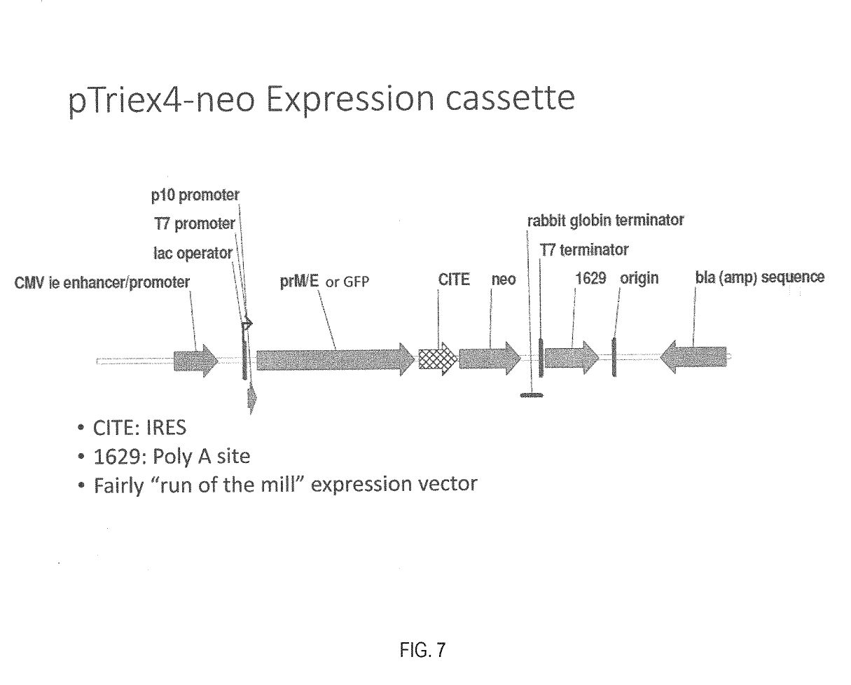

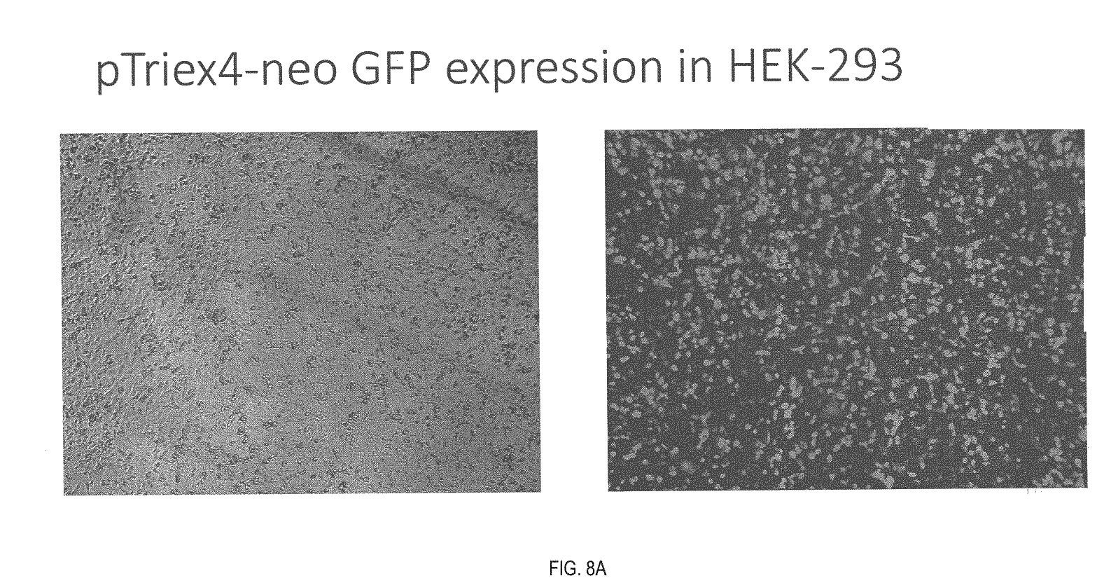

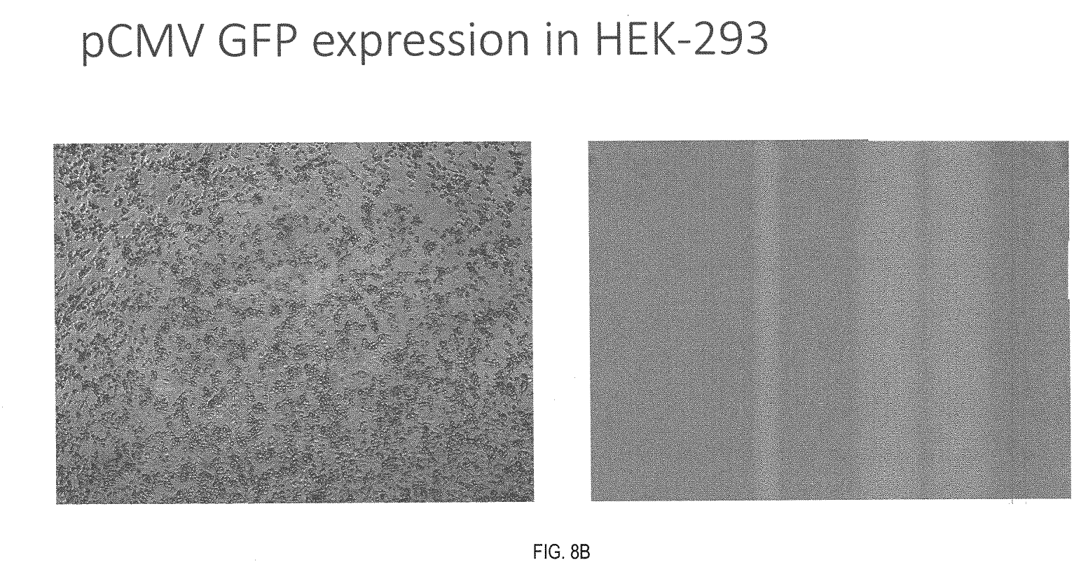

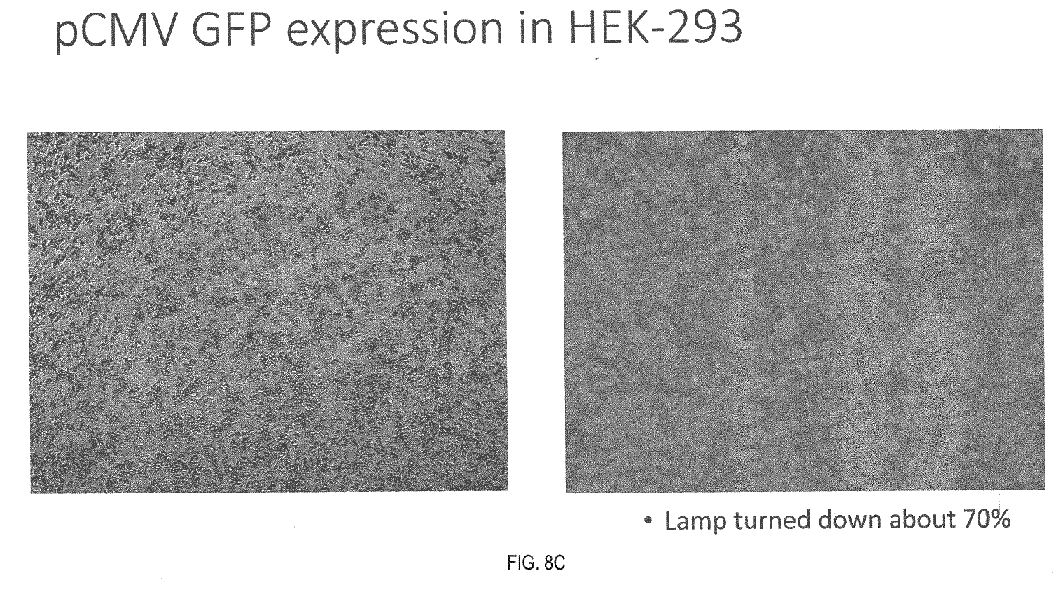

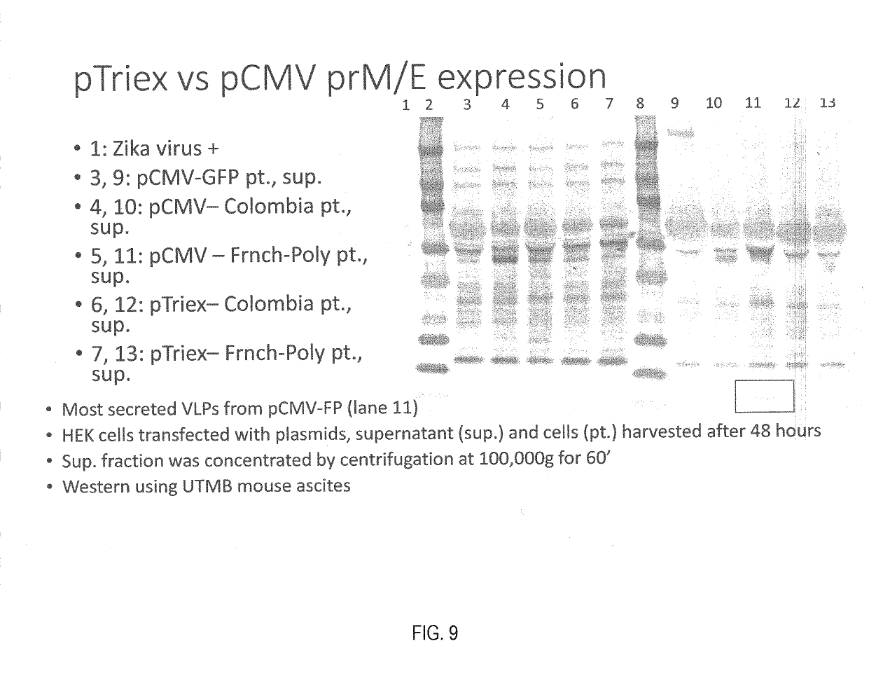

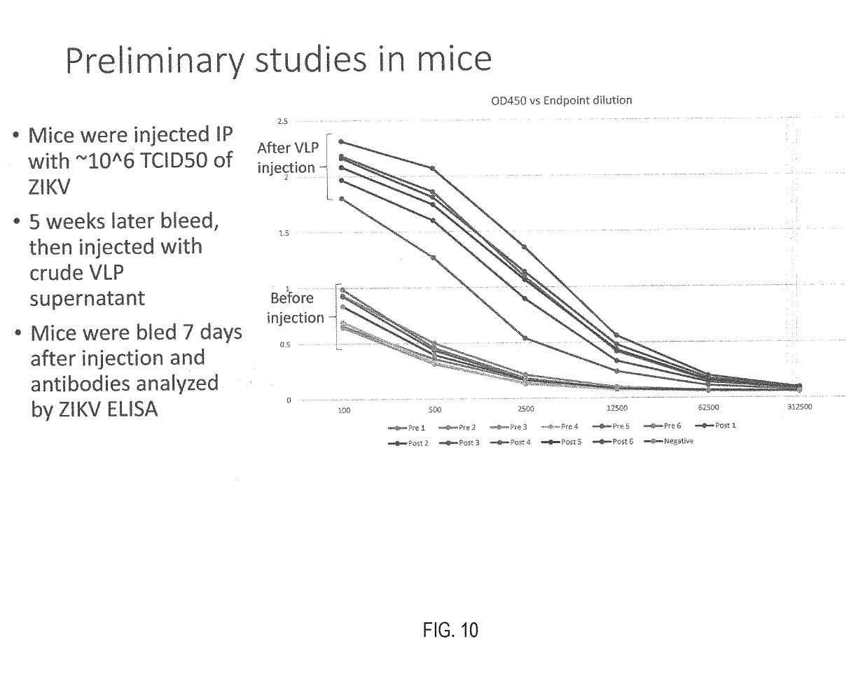

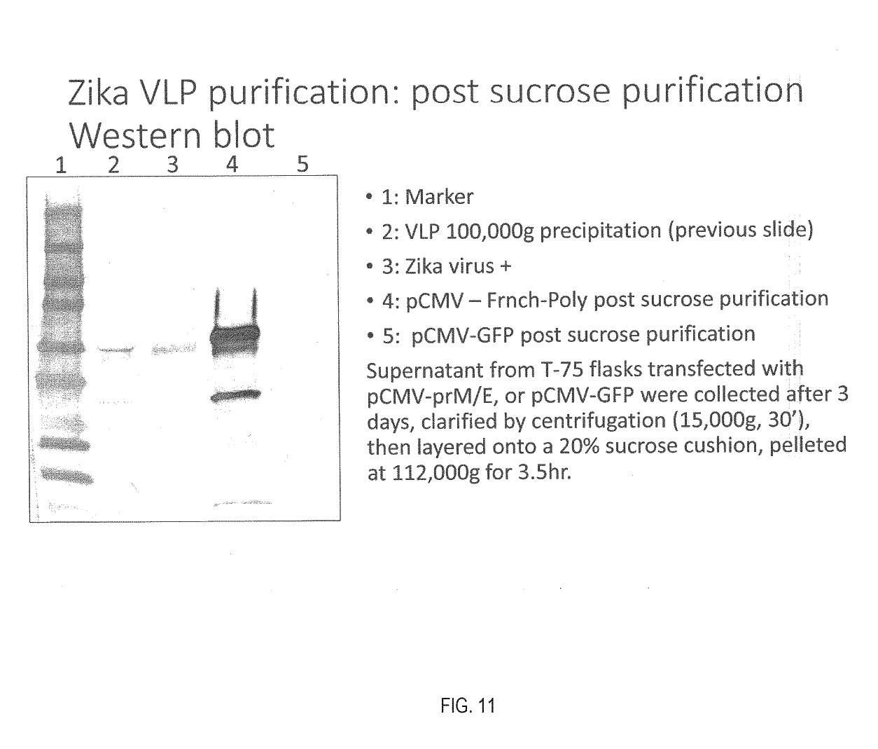

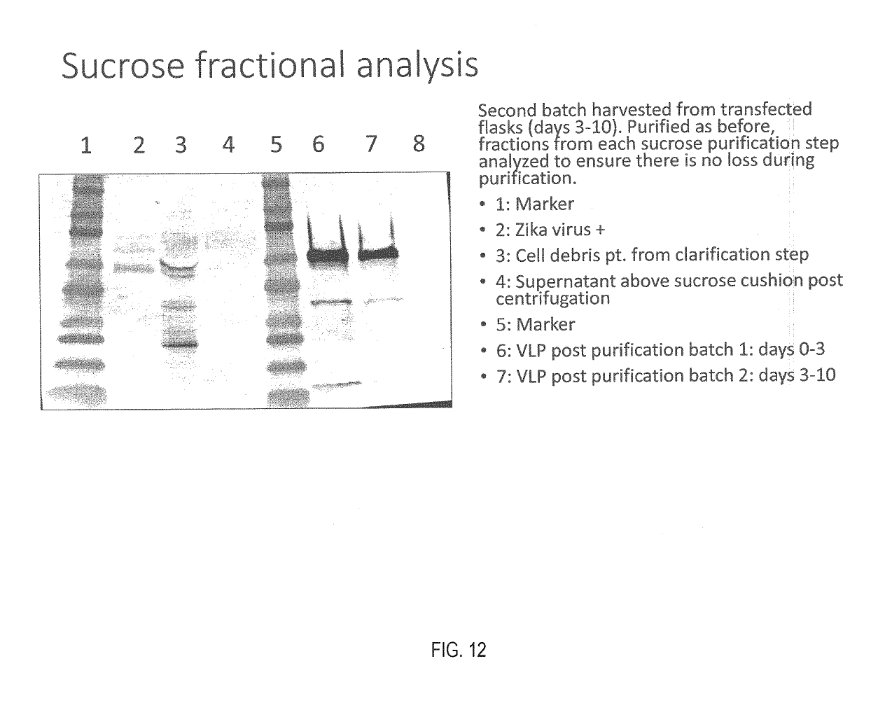

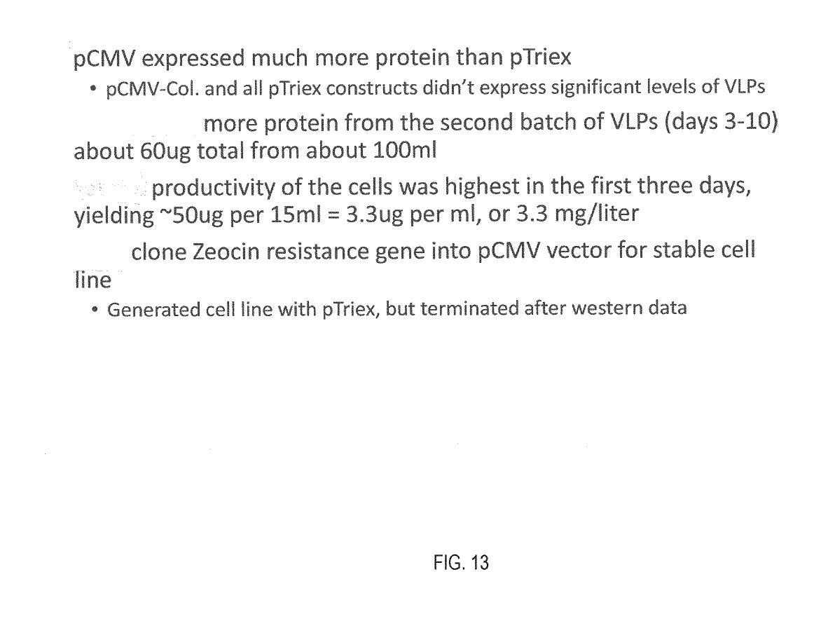

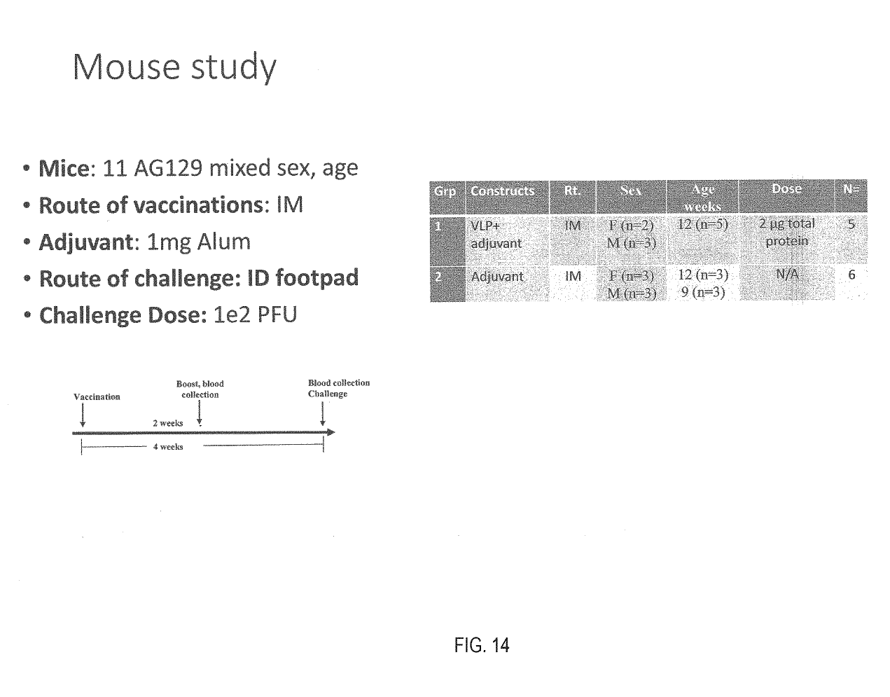

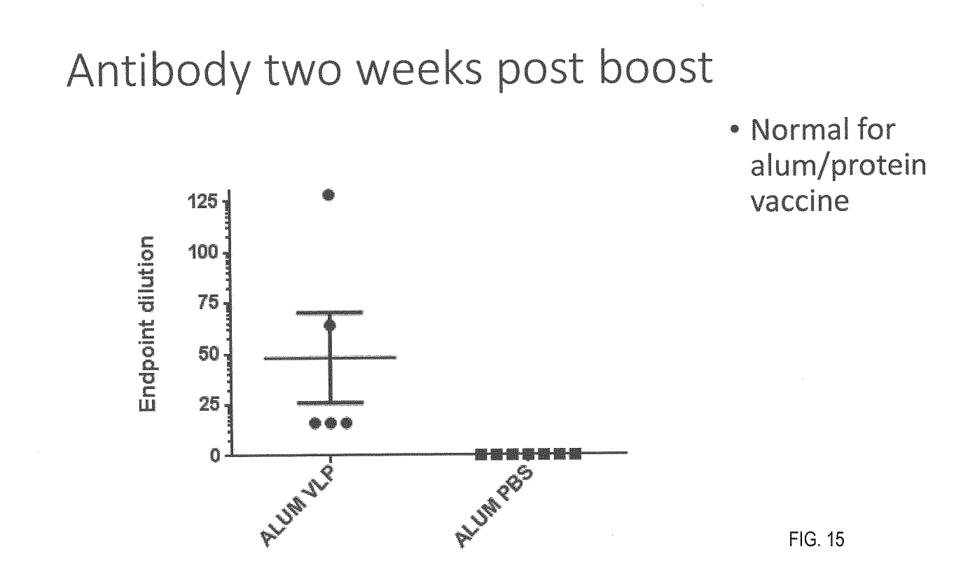

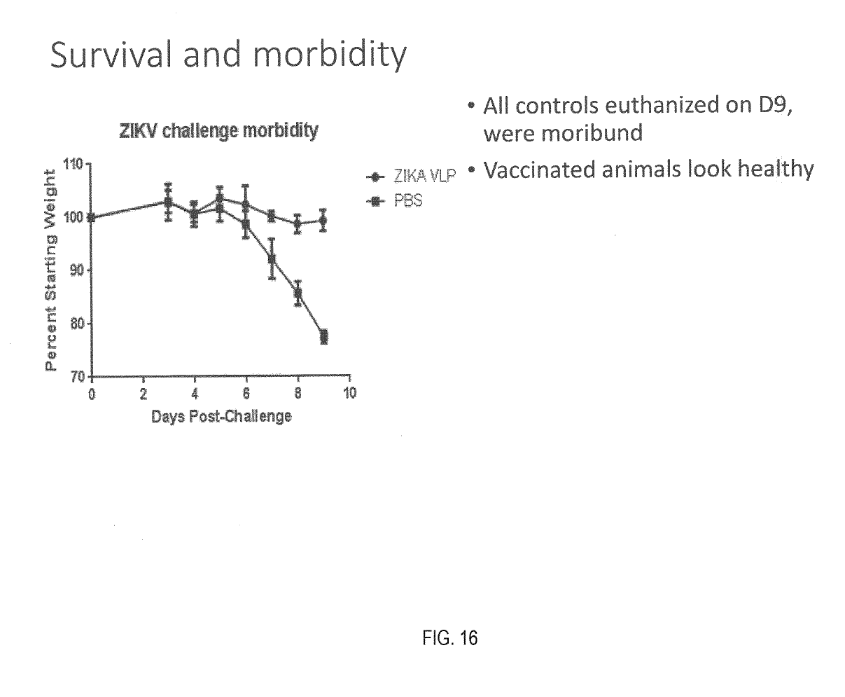

Exemplary vectors expressing GFP were transfected into HEK293 cells and expression was assessed (FIGS. 7-8). prM/E sequences were also expressed from the two vectors in HEK cells and supernatants and cells analyzed 48 hours later (FIG. 9). Supernatants were concentrated by centrifugation at 100,000 g for 60 minutes. Western blots were analyzed using University of Texas Medical Branch (UTMB) mouse ascites. More VLPs were secreted from pCMV-FP transfected cells (lane 11 in FIG. 9) than pTriex transfected cells (lane 13). Sucrose purified fractions were subjected to Western blot (FIGS. 10-11). pCMV-prM/E SC purified pellet (pt) appeared to contain high levels of E protein while pCMV-GFP pt did not, indicating that staining was specific to expression of prM and E genes. In summary, a pCMVvector expressed more protein than a pTriex vector. VLPs collected at days 3-10 provided for about 60 μg total protein from about 100 mL. On day 3 the productivity of the cells was about 50 μg per 15 mL (3.3 μg per mL, or 3.3 mg/L). For stably transfected cells, a marker, e.g., a Zeocin resistance gene, may be introduced into the vector that expresses prM/E.

ZIKV VLPS (ZIKVLPs) formulated with alum were injected into 6-8-week-old interferon deficient A129 and AG129 mice. Control mice received PBS/alum. Animals were challenged with 200 PFU (>400 LD₅₀s) of ZIKV strain H/PF/2013. All vaccinated mice survived with no morbidity or weight loss while control animals either died at 9 days post challenge (AG129) or had increased viremia (A129). Neutralizing antibodies were observed in all ZIKVLP vaccinated mice.

### EXAMPLE 4

### Materials and Methods

### Cells and Viruses

African Green Monkey kidney cells (Vero) and Human embryonic kidney 293 (HEK293) were obtained from ATCC (ATCC; Manassas, Va., USA) and grown in Dulbecco's modified Eagle medium (DMEM) supplemented with 10% fetal bovine serum (FBS; Hyclone, Logan, Utah), 2 mM L-glutamine, 1.5 g/l sodium bicarbonate, 100 U/ml of penicillin, 100 μg/ml of streptomycin, and incubated at 37° C. in 5% CO2. ZIKV strain H/PF/2013 (GenBank:KJ776791), was obtained from Xavier de Lamballerie (European Virus Archive, Marseille France). Virus stocks were prepared by inoculation onto a confluent monolayer of Vero cells.

### Animals

Mice of the 129/Sv background deficient in alpha/beta interferon alpha/beta/gamma (IFN-α/β/IFN-Υ) receptors (AG129 mice) were obtained from B&K Universal Limited (Hull, England) and were bred in the pathogen-free animal facilities of the University of Wisconsin-Madison School of Veterinary Medicine. 5-week-old BALB/c mice (The Jackson Laboratory, Maine, USA) were used for wild-type vaccination studies. Groups of mixed sex mice were used for all experiments.

### Production and Purification of ZIKV VLPs

The prM and E genes of ZIKV strain H/PF/2013 with nascent signal sequence were cloned into a pCMV expression vector under the control of a cytomegalovirus (CMV) promoter and CMV polyadenylation signal (pCMV-prM/E, FIG. 1). Endotoxin free, transfection grade DNA was prepared using Maxiprep kit (Zymo Research, Irvine, Calif.). VLPs were expressed by transfecting 90% confluent monolayers of HEK293 cells in a T-75 flasks with 15 μg of pCMV-prM/E using Fugene HD (Promega, Madison, Wis.) transfection reagent according to manufacturer protocol. The 10 ml supernatant was harvested 72 hr after transfection, and clarified by centrifugation at 15,000 RCF for 30 min at 4° C. Clarified supernatants were layered onto a 20% sucrose cushion and ultra-centrifuged in a SW-28 rotor at 112,000 RCF for 3.5 hours at 4° C. Pellet (PT) and supernatant (SUP.) fractions at each step were saved for analysis by SDS-PAGE and Western blot. Post sucrose cushion PT were resuspended in Phosphate Buffered Saline (PBS) pH 7.2. Total protein in VLP preparations was quantified by Bradford assay. VLP specific protein was determined by comparing Zika specific bands on SDS-PAGE gels to known concentrations of BSA using ImageJ software.

### Western Blot

VLP fractions were boiled in Laemmli sample buffer (BioRad, Hercules, Calif., USA) and resolved on a 4-20% SDS-PAGE gel (Biorad) by electrophoresis using a Mini-PROTEAN 3 system (BIO-RAD, CA). Gels were electroblotted onto nitrocellulose membranes using a Turboblot® system. Membranes were blocked in 5% (W/V) skim milk and probed with mouse hyper immune ascites fluid primary antibody (1:5000) and goat anti-mouse HRP conjugated secondary antibody (1:5000). Membranes were developed using a solid phase 3,3′,5,5′-tetramethylbenzidine (TMB) substrate system.

### Transmission Electron Microscopy

Samples were negatively stained for electron microscopy using the drop method. A drop of sample was placed on a Pioloform™ (Ted Pella, Inc.) carbon-coated 300 Mesh Cu grid, allowed to adsorb for 30 seconds, and the excess removed with filter paper. Next, a drop of methylamine tungstate or uranyl acetate (Nano-W, Nanoprobes Inc.) was placed on the still wet grid, and the excess removed. The negatively stained sample was allowed to dry, and was documented in a Philips CM120 (Eindhoven, The Netherlands) transmission electron microscope at 80 kV. Images were obtained using a SIS MegaView III digital camera (Soft Imaging Systems, Lakewood. Colo.).

### Vaccination and Viral Challenge

Each of the following animal studies was performed as one biological replicate. For VLP formulations, the indicated dose of sucrose cushion purified VLPs was mixed with 0.2% Imject Alum (Thermo Scientific) according to manufacturer's protocol. Groups of AG129 mice were injected intramuscularly (TM) with VLPs mixed with alum (n=5) or PBS mixed with alum (n=6) at 6 weeks of age, and again at 8 weeks of age. Sub-mandibular blood draws were performed pre boost and pre challenge to collect serum for analysis by neutralization assays and for passive transfer studies.

AG129 mice were challenged with 200 PFU of ZIKV strain H/PF/2013 in 25 μL volumes by intraderml (ID) injection into the right hind footpad at 11 weeks of age. Balb/c mice were vaccinated once at 5 weeks of age as above, and challenged at 13 weeks of age with 200 PFU of H/PF/2013 in 50 μl by retro orbital injection (IV route).

Following infection, mice were monitored daily for the duration of the study. Mice that were moribund or that lost greater than 20% of starting weight were humanely euthanized. Sub-mandibular blood draws were performed on day two post challenge (PC) and serum collected to measure viremia.

Eight week old AG129 mice were used for passive transfer studies Five naive mice were injected intraperitoneally (IP) with 500 μL of pooled serum from VLP vaccinated, diluted serum (1:5 n=4, 1:10, n=4), or serum from PBS/alum (n=5) treated mice. At 12 h post transfer, mice were challenged with 20 PFU in 25 μl as above.

### Viremia Assays

Viremia was determined by TCID₅₀ assay. Briefly, serum was serially diluted ten-fold in microtiter plates and added to duplicate wells of Vero cells in 96-well plates, incubated at 37° C. for 5 days, then fixed and stained with 10% (W/V) crystal violet in 10% (V/V) formalin. Plates were observed under a light microscope to determine the 50% tissue culture infective doses (TCID₅₀s). Serum samples were also tested for viral RNA copies by qRT-PCR. RNA was extracted from 0.02ml of serum using the ZR Viral RNA Kit (Zymo Research, Irvine, Calif.). Viral RNA was quantified by qRT-PCR using the primers and probe designed by Lanciotti et al (Lanciotti et al., 2008). The qRT-PCR was performed using the iTaq Universal Probes One-Step Kit (BioRad, Hercules, Calif.) on an iCycler instrument (BioRad, Hercules, Calif.). Primers and probe were used at final concentrations of 500 nM and 250 nM respectively. Cycling conditions were as follows: 50° C. for 10 min and 95° C. for 2 min, followed by 40 cycles of 95° C. for 15 sec and 60° C. for 30 sec. Virus concentration was deteif lined by interpolation onto an internal standard curve made up of a 5-point dilution series of in vitro transcribed RNA, with the lowest copies per reaction being 100.

### Neutralization Assay

Serum antibody titers were determined by microneutralization assay. Briefly, serum was incubated at 56° C. for 30 min to inactivate complement and then serially diluted two-fold in microtiter plates. 200 PFUs of vines were added to each well and incubated at 37° C. for 1 h. The virus-serum mixture was added to duplicate wells of Vero cells in 96-well plates, incubated at 37° C. for 5 days, then fixed and stained with 10% (W/V) crystal violet in 10% (V/V) formalin, then observed under a light microscope. The titer was determined as the serum dilution resulting in the complete neutralization of the virus.

### Plaque Reduction Neutralization Test

Serum samples were serially diluted, mixed with 200 PFU of the ZIKV H/PF/2013 strain and incubated for 1 hr at 37° C. This serum/virus mixture was added to confluent layers of Vero cells in 96 well plates and incubated for 1 hr at 37° C., after which the serum/virus mixture was removed and overlay solution (3% CMC, 1×DMEM, 2% FBS and 1×Anti/Anti) was added. After 48 hrs of infection, the monolayers were fixed with 4% PFA, washed twice with PBS, and then incubated with ZIKV hyperimmune mouse ascitic fluid (1:2000, UTMB) diluted in blocking solution (1×PBS, 0.01% Tween-20 and 5% Milk) and incubated overnight at 4° C. Plates were washed three times with PBS-T and then peroxidase-labeled goat anti-mouse secondary antibody (1:2000) was incubated on monolayers for 2 hours at 37° C. Following incubation, cells were washed a final three times with PBS-T and developed using 3-amino-9-ethylcarbazole (AEC)-peroxidase substrate. The amount of formed foci were counted using an ELISPOT plate reader (ImmunoSPOT-Cellular Technology); quality control was performed to each scanned well to ensure accurate counting. Neutralization percentages (Nx) were calculated per sample/replicate/dilution as follows:

$$Nx = \{ 100 - [ 100 ( \frac{A}{Control} )$$

Where A corresponds to the amount of foci counted in the sample and Control is the geometric mean of foci counted from wells treated with cells and virus only. Data of corresponding transformed dilutions (Log(1/Dilution)) against neutralization percentages per sample was plotted and fitted to a sigmoidal dose-response curve to interpolate PRNT₅₀ and PRNT₉₀ values (GraphPad Prism software).

### RESULTS

Expression and Purification of Soluble, Zika VLPs To generate Zika VLPs (ZIKVLPs), we cloned the prM/E genes with native signal sequence into a pCMV expression vector (pCMV-prM/E) (FIG. 1A), transfected HEK293 cells and harvested supernatants (supe.) 3 days post transfection. 78 μg total protein was recovered from post sucrose purification of which 21.6 μg was ZIKVLP protein. Western blot analysis of this pCMV-prM/E supe. revealed expression of an about 50 kDa size band (FIG. 1B, lane 2) that corresponded in size to the predicted size of the Zika virus E gene, and additionally matched positive control Zika virus stocks (FIG. 1B, lane 3). To test the hypothesis that expression of Zika prM and E genes spontaneously form extracellular particles, supernatants from pCMV-prM/E and pCMV-GFP (negative control) transfected cells were centrifuged on a sucrose cushion (SC) sufficient for pelleting of flavi virus particles from cell culture proteins (Merino-Ramos et al., 2014). pCMV-prM/E SC purified pellet (pt.) appeared to contain high levels of E protein, indicating that staining was specific to expression of prM and E genes. To determine if the immune reactive extracellular particles were virus like in nature, we performed transmission electron microscopy (TEM) on pCMV-prM/E SC pt. material. TEM revealed virus like particles with a size that ranged from 30-60 nm, and a typical size of about 50 nm (FIGS. 1C-E).

### Administration of ZIKVLPs is Immunogenic and Protects Highly ZIKV Susceptible α/β/γ Interferon Deficient (AG129) Mice

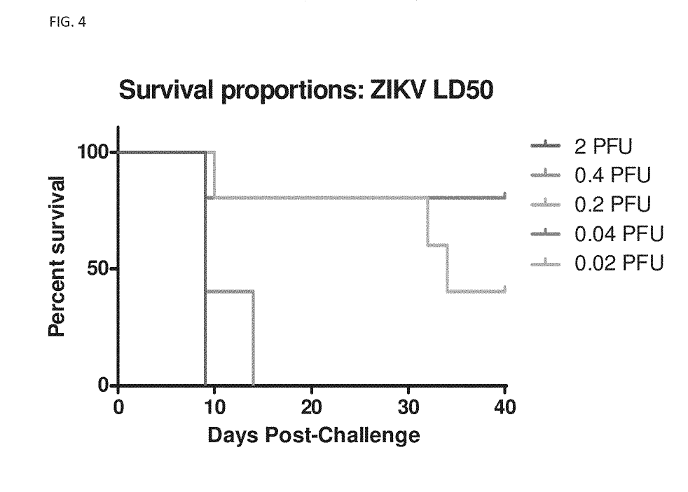

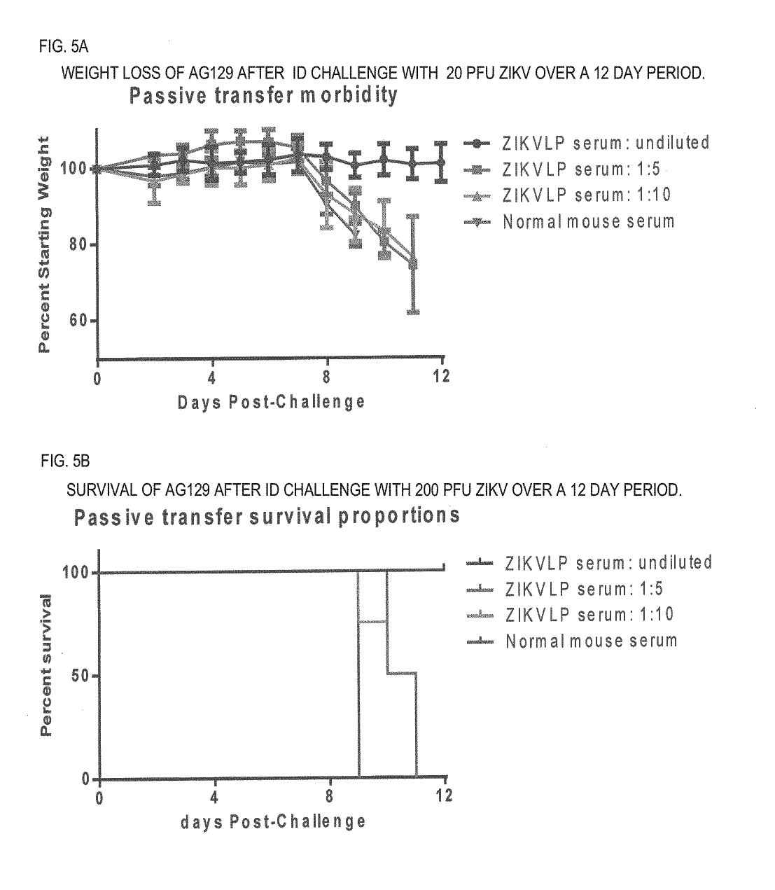

First, the LD50 of the H/PF/2013 strain in 12 week-old mixed sex AG129 mice was determined. Groups of mice (n=5) were infected with 5-fold serial dilutions from 2 PFU to 0.02PFU of ZIKV and monitored for 4 weeks following the last mortality. All mice infected with 2 or 0.4 PFU died within the first week of challenge (FIG. 4), while lower doses killed only 1 to 2 mice within the first two weeks. Interestingly, 2 mice infected with 0.2 PFU ZIKV became ill and were euthanized due to weight loss and paralysis 4.5 weeks following challenge. The resultant LD₅₀ value in PFUs was calculated to be 0.19 PFU by the Reed-Muench (REED and MUENCH, 1938) method.

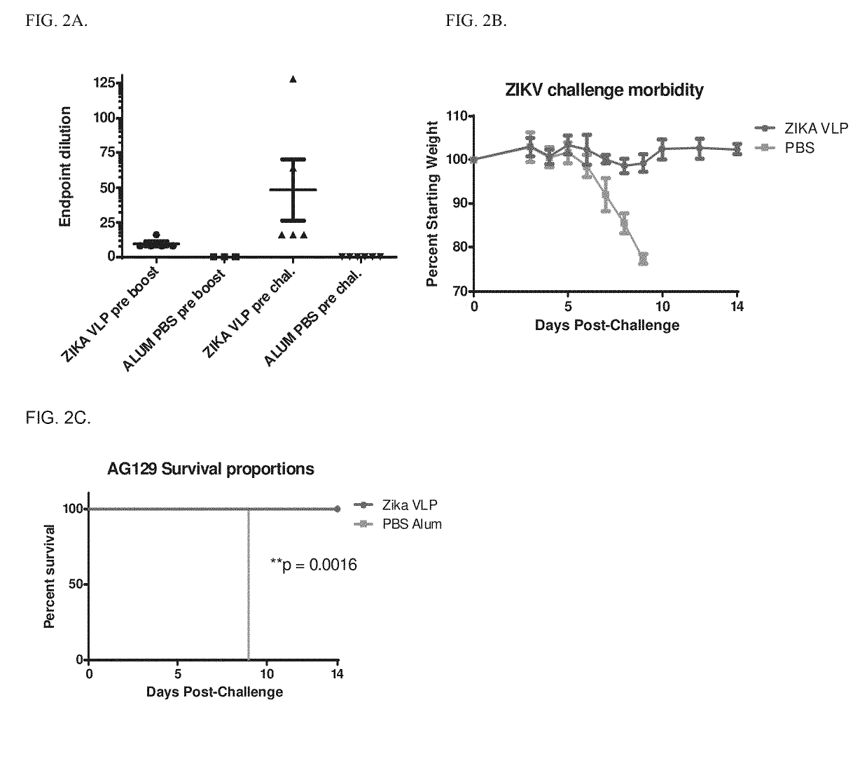

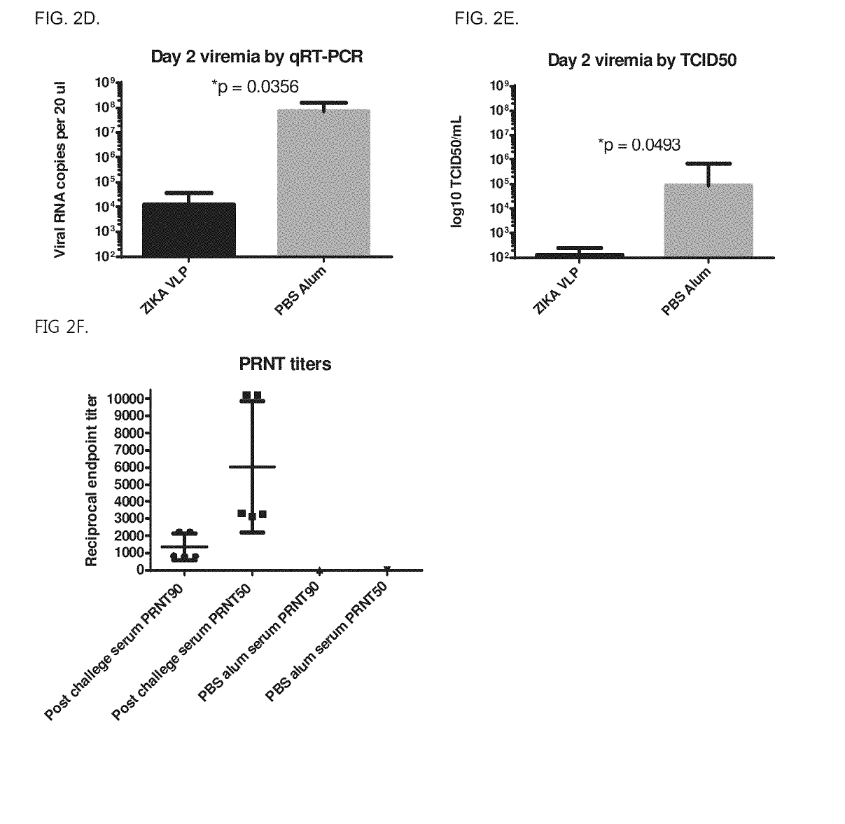

To determine if ZIKVLPs are immunogenic and protective in highly susceptible AG129 mice, groups of mice received a prime and boost of 450ng ZIKVLPs. AG129 mice that received ZIKVLPs developed low levels (GMT=1:9.2) of neutralizing antibodies (nAbs) at two weeks post administration (FIG. 2A), that increased two weeks after boost (GMT=1:32). Five weeks after primary vaccination, all mice were challenged with 200 PFU (>1000 LD₅₀s) of ZIKV by the ID route. Mice administered ZIKVLPs maintained weight, while mice that received PBS/alum experienced significant morbidity throughout the challenge period (FIG. 20B). All control mice (survival 0/6) died 9 days after ZIKV challenge and had significantly lower survival (p=0.0016) than mice administered ZIKVLPs (survival 5/5, FIGS. 2B and C). Finally. ZIKVLPs vaccinated mice had significantly lower levels of viremia on day 2 post challenge than control mice detected by qRT-PCR (ZIKVLP=1.3×10⁴ RNA copies, PBS/alum 9.6×10⁷ RNA copies, p=0.0356, FIG. 2D) and TCID50 assay (ZIKVLP=1.3×10² TCID50s, PBS/alum 2.8×10⁵ TCID₅₀s p=0.0493, FIG. 2E).

### ZIKVLPs Elicit Plaque Reducing Neutralizing Antibody Titers in Mice That Can Be Passively Transferred to Naïve Mice.

The plaque reduction neutralization test (PRNT) assay is widely considered to be the “gold standard” for characterizing and quantifying circulating levels of anti-dengue and other flaviviral neutralizing antibodies (nAb) (Thomas et al., 2009). A PRNT assay was developed for rapidly measuring ZIKV specific neutralizing antibodies. Pooled serum samples collected from mice pre-challenge, as well as individual serum samples collected from mice post-challenge were tested by this PRNT assay. Pre challenge, pooled serum from mice administered ZIKVLPs had a calculated 50% plaque reduction (PRNT₅₀) titer of 1:157. The PRNT₅₀ titer increased 2 weeks post challenge (GMT=5122) (FIG. 2F).

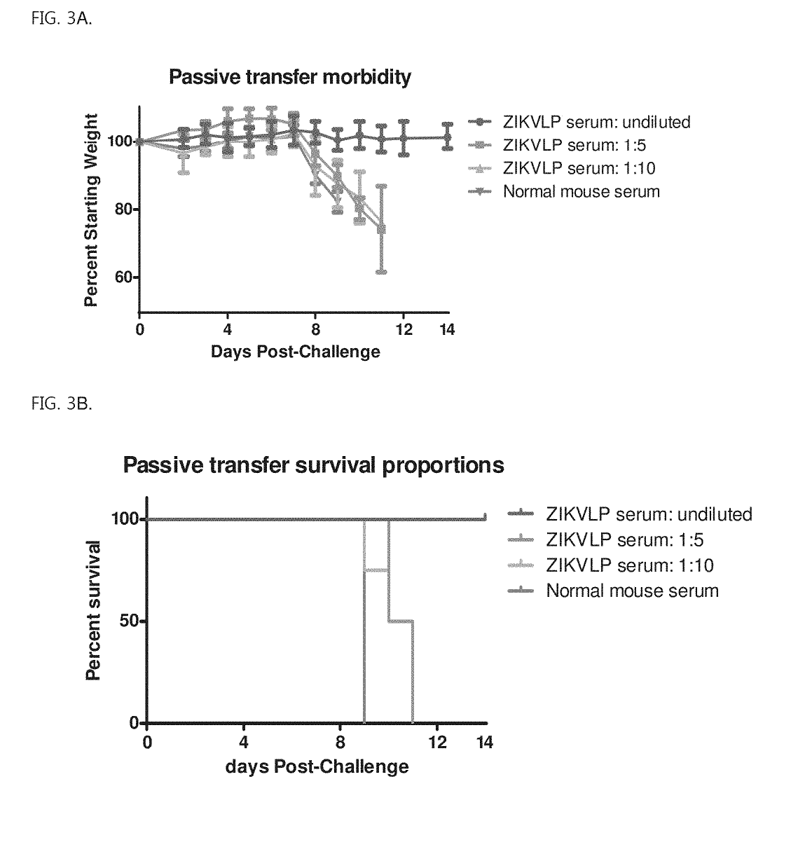

To test the role of anti-ZIKV antibodies in protection against challenge, groups of mice received ZIKVLP antiserum (pooled pre challenge serum, titer in FIG. 2F), undiluted (n=5), diluted 1:5 (n=4), or 1:10 (n=4). As a negative control, mice (n=5) were transferred serum from mice previously vaccinated with PBS alum. Negative control mice rapidly lost weight starting after day 7 and all died day 9 post challenge (FIGS. 3A-B). Mice that received undiluted serum maintained weight throughout the 14 day period post challenge, and showed no signs of infection. Mice that received diluted anti-ZIKV antibodies were not protected from challenge, although survival and weight loss were slightly extended relative to negative control mice (FIGS. 3A-B).

### A Single Dose of ZIKVLPs Can Protect Highly Susceptible AG129 Mice

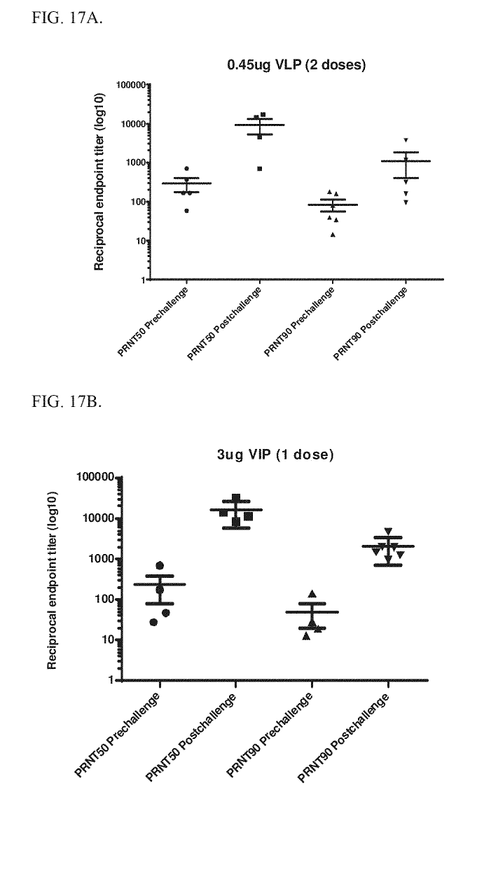

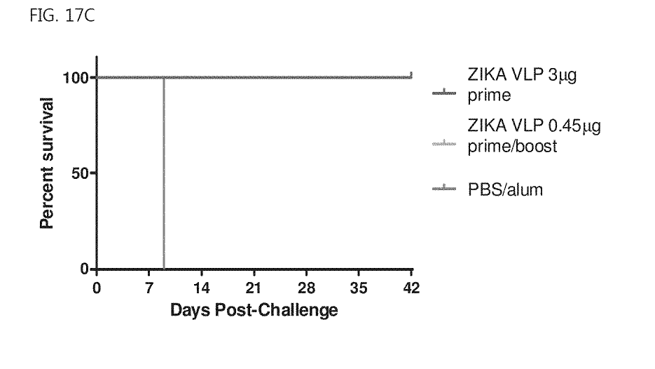

To determine if a single dose could protect AG129 mice, groups of 6-week old AG129 mice were vaccinated with 3 μg ZIKVLPs adjuvanted with alum. An additional group of mice (n=5) was vaccinated with a prime and boost of 0.45 μg adjuvanted with alum for comparison. Negative control mice (n=5) received a prime and boost of PBS/alum. Vaccinated mice developed neutralizing antibodies measured by PRNT assay prior to challenge (FIG. 17A). Eight weeks following primary vaccination mice were challenged with 200 PFU (>1000LD₅₀s) of ZIKV by the ID route. All mice administered a prime of 3 μg or a prime and boost of 0.45 μg ZIKVLPs survived throughout the 6 week challenge period (FIG. 17C) and maintained weight throughout the challenge period. Pre challenge neutralizing antibody titers in both single (GMT PRNT50=288, PRNT90=81) and double dose (GMT PRNT50=235, PRNT90=50) groups increased significantly (p<0.005) in all animals measured at 3 weeks post challenge (FIGS. 17A-B).

### ZIKVLPS Protect Wildtype BALB/c Mice

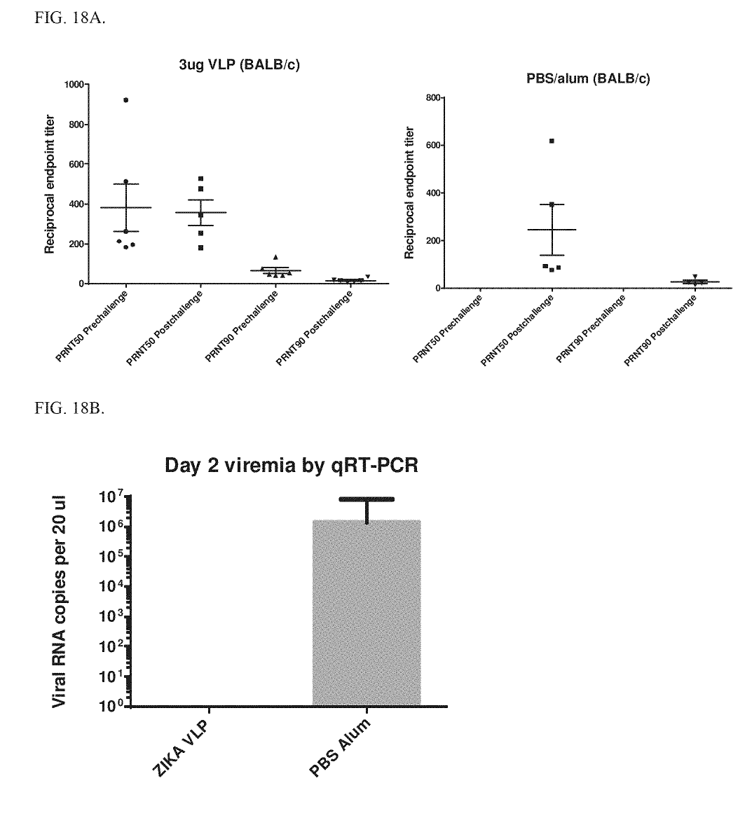

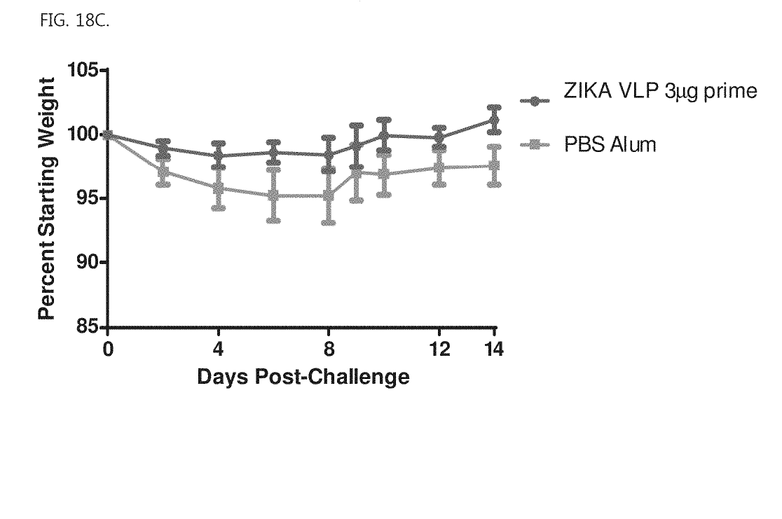

To determine if ZIKVLPs can protect wildtype BALB/c mice against non-lethal ZIKV challenge, a group (n=6) was vaccinated with a single dose of 3 ZIKVLPS adjuvanted with alum. Negative control mice (n=5) were administered PBS/alum. Eight weeks after vaccination mice were challenged with 200 PFU ZIKV by the IV route. A single dose of ZIKVLPs elicited high titers of neutralizing antibodies (PRNT50=381, PRNT90=75) detected immediately prior to challenge (FIG. 22A). Mice vaccinated with ZIKVLPS were completely protected from viremia on day 2 post challenge (FIG. 18B), and maintained weight throughout the challenge period (FIG. 18C). Negative control animals lost minor amounts of weight beginning at day 2 post challenge, had high levels of viremia and recovered by 2 weeks post challenge. Neutralizing antibodies were undetectable in negative control mice prior to challenge, but increased significantly after challenge (FIG. 18A). Antibody titers in vaccinated mice decreased, but were not significantly different than before ZIKV challenge (FIG. 18A).

### DISCUSSION

Most experts and public health workers agree that a Zika vaccine is urgently needed. In February 2016, the World Health Organization declared that the recent clusters of microcephaly and other neurological disorders in Brazil constitute a public health emergency of international concern. Their recommendations included enhanced surveillance and research, as well as aggressive measures to reduce infection with Zika virus, particularly amongst pregnant women and women of childbearing age. ZIKV is now receiving considerable attention due to its rapid spread in the Americas, and its association with microcephaly (Mlakar et al., 2016) and Guillain-Barre syndrome (Pinto Junior et al., 2015). In these studies, a ZIKV-virus-like particle (VLP) vaccine was designed and it was expressed in vitro as shown by western blot and transmission electron microscopy, and its protective efficacy and role of antibodies in protection in the AG129 mouse model tested. An overall yield of 2.2 mg/L was calculated for the VLP tested. Similar expression levels have been reported for other flavivirus VLP expression strategies (Pijlman, 2015). Future work will optimize VLP production and purification parameters, which should significantly increase both yield and purity. Stably transfected HEK cells that continuously express VLPs allow for scalable production to help meet global demand for a ZIKV vaccine, which is estimated to be 100 million doses a year.

ZIKV-VLPs, formulated with alum, induced detectable neutralizing antibodies and protected animals against lethal challenge (>400 LD₅₀s) with no morbidity or mortality. Pre-challenge GMT neutralizing titers were 1:32, and pooled pre-challenge serum PRNT₉₀ and PRNT₅₀ titers were 1:34 and 1:157 respectively. At a relatively low dose of 450 ng, our results indicate that our ZIKVLPs are highly immunogenic. The antibody titers obtained are consistent with those reported for other highly immunogenic flavivirus VLP vaccines (Ohtaki et al., 2010; Pijlman, 2015). Previous work has shown a direct correlation between dose of VLPs and neutralizing antibody titers. For ZIKV, questions remain about the quantitative relationship between dose of VLPs and their effect on neutralizing antibody titers and protection from ZIKV challenge in vivo.

In the above-described studies, mice were vaccinated with ZIKVLPS and challenged with a homologous strain of ZIKV (H/PF/2013), which raises the question of ZIKVLP specific antibody cross reactivity to heterologous viruses currently circulating in the Americas. Although the H/PF/2013 virus was isolated well before the current outbreak from a patient infected in French Polynesia, there is a high degree of amino acid similarity (about 99%) to endemic South American strains of ZIKV (Faria et al., 2016; Zanluca et al., 2015). Some experts agree that the high serological cross-reactivity among ZIKV strains would allow for a monovalent vaccine (Lazear and Diamond, 2016). Nevertheless, care must be taken to empirically determine if antibody responses elicited by ZIKV LPs cross-react and protect against South American strains. Finally, any future ZIKV vaccination programs should incorporate careful surveillance of circulating strains to help suppress immunological escape, and ensure efficacy of vaccines in human populations.

Vaccinated AG129 mice challenged with >1000 LD₅₀s had low levels of viremia (1.3×10² TCID₅₀s, FIG. 2E) detected after challenge. Copies of RNA ZIKV genomes in serum of mice were significantly higher than levels of viremia. However, the disparity between viral genome copies and viremia has been observed for other flaviviruses including dengue (Bae et al., 2003). Since AG129 mice are highly susceptible to viral challenge, it is possible that the challenge dose given for the active vaccination study was artificially high. Methods for challenging mice from infected mosquito bite should be developed to most accurately mimic natural infection. The most important criteria for any ZIKV vaccine is its ability to prevent placental and fetal pathology in ZIKV infected pregnant women. Recently developed IFN deficient pregnant mouse models can provide an opportunity to assess if vaccination of pregnant animals can protect the fetus from ZIKV-induced pathology. (Miner et al., 2016). Although models for ZIKV infection in pregnant non-human primates (NHP) are still being developed, ZIKV vaccines should be tested in NHP translational models which most accurately mimics human immune responses to vaccination.

A VLP vaccine approach against ZIKV has significant advantages over other technologies as it will eliminate concerns of live attenuated vaccines and insufficient inactivation of killed vaccines for pregnant women and other populations at high risk of suffering the devastating effects of ZIKV infections. Production of inactivated vaccines requires high titer growth of infectious virus which may pose a safety concern for workers. Additionally, the production of both attenuated and inactivated ZIKV vaccines is limited to “batch” production, whereas flavirus VLPs can continuously expressed from stable cell lines. In recent years, recombinant virus-like particle (VLP)-based vaccine strategies have been frequently used for vaccine design. VLPs are known to be highly immunogenic and elicit higher titer neutralizing antibody responses than subunit vaccines based on individual proteins (Ariano et al., 2010).

The role of neutralizing antibodies in protecting against ZIKV was demonstrated by antibody passive transfer studies as naive AG129 mice receiving pooled serum from VLP vaccinated animals were fully protected. These results are consistent with previous findings that indicate the important role of antibodies in protecting against many insect-borne flaviviruses, such as Japanese encephalitis, west Nile virus, and tick borne encephalitis (Chiba et al., 1999; Kimura-Kuroda and Yasui, 1988; Tesh et al., 2002), even at low levels of circulating antibodies. In this study, full protection was observed when animals received undiluted serum (PRNT₅₀ 1:157), with no weight loss or other clinical signs observed. While these studies highlight the importance of serum antibodies in ZIKV protection, there are still many important questions related to ZIKV immunology. What is the minimum antibody titer needed for protection, do ZIKVLPs elicit CD8+ responses and are these responses involved in protection, and what is the overall role of cellular immunity in protection? It is also important to determine if anti-ZIKV antibodies, particularly those elicited by ZIKVLPs, play any role in dengue protection or disease enhancement.

In this study AG129 IFN receptor-deficient mice were used. This mouse models are commonly used for the evaluation of arboviral vaccines, including dengue, chikungunya and yellow fever virus (Meier et al., 2009; Partidos et al., 2011; Prestwood et al., 2012). We recently documented the suitability of mice deficient in IFN-α/β and -γ receptors as an animal model for ZIKV, as they are highly susceptible to ZIKV infection and disease, developing rapid viremic dissemination in visceral organs and brain and dying 7-8 days post-infection (Aliota et al., 2016), and evaluated doses as low as 1 PFU. In our current studies we observed consistent lethality at doses below 1 PFU, indicating that there are viral subpopulations refractory for the formation of CPE in cell culture, but still capable of establishing a lethal infection in highly susceptible mice. It is of great interest is that at a very low dose (0.2PFU) two of five mice became ill more than 1 month after infection, as infection with ZIKV typically produces rapid lethality in AG129 mice.

The current studies challenged mice with 200 PFU at 11 weeks of age. All control mice lost 20% weight, were moribund, and succumbed to by challenge by day 9. ZIKV challenge therefore appears to be completely lethal in both juvenile and adult AG129 mice. The AG129 mouse model exhibits an intact adaptive immune system, despite the lack of an IFN response, and it has been used extensively to evaluate vaccines and antivirals for DENV (Brewoo et al., 2012; Fuchs et al., 2014; Johnson and Roehrig, 1999; Sarathy et al., 2015). In our studies WT BALB/c mice did not succumb to infection with ZIKV consistent with previous studies where BALB/c mice were experimentally inoculated with 200 PFU of ZIKV (Larocca et al., 2016). Mice also developed high levels of viremia following IV inoculation. A single dose of VLPs prevented detection of viral RNA copies in serum of vaccinated mice at 2 days post infection—when viremia levels typically peak in the BALB/c model. It is possible that viral replication was completely inhibited, as there was no “boost” response in neutralizing antibodies observed following challenge. Finally, in repeat AG129, and Balb/c mice mouse studies, animals were protected from ZIKV challenge 8 weeks after vaccination. ZIKVLP therefore appear to elicit a potent “memory” response.

In the present study, aluminum hydroxide (commonly known as alum) was used as the adjuvant for ZIKV-VLP preparations. Since its first use in 1932, vaccines containing aluminum-based adjuvants have been successfully administered in humans demonstrating excellent safety. Adjuvant formulations of ZIKV-VLP may facilitate antigen dose sparing, enhanced immunogenicity, and broadened pathogen protection.

In summary, a vaccine against ZIKV is currently unavailable, nor is there any specific prophylactic treatment. A VLP based Zika vaccine that elicits protective antibodies in mice, and is safe, suitable for scalable production, and highly immunogenic, is disclosed herein. Fast-tracking development of this ZIKV vaccine is a public health priority and is crucial for restoring confidence and security to people who wish to have children or reside in, or visit areas in which ZIKV is endemic.

### REFERENCES

Akahata et al., Nat. Med. 16:334 (2010).
Aliota et al., PLoS Negl. Trop. Dis., 10:e0004682 (2016).
Ariano et al., CMAJ, 182:357 (2010).
Bae et al., J. Virol. Methods, 110:185 (2003).
Brewoo et al., Vaccine, 30:1513 (2012).
Butler, Nature, 531:153 (2016).
Cao-Lormeau et al., Emerg. Infect. Dis., 20:1085 (2014).
Chiba et al., Vaccine, 17:1532 (1999).
Duffy et al., N. Engl. J. Med., 360:2536 (2009).
Dyer, BMJ. 351:h6983 (2015).
Faria et al., Science, 352:345 (2016).
Faye et al., PLoS Negl. Trop. Dis., 8:e2636 (2014).
Fuchs et al., Vaccine, 32:6537 (2014).
Gaskell et al., Emerg. Infect. Dis., 23:137 (2017).
Gulland, BMJ, 352:i657 (2016).
Hennessey et al., Am. J. Trop. Med. Hyg., 95:212 (2016).
Hombach et al., Bmj, 355:i5923 (2016).
Honein et al., Jama, 317:59 (2017).
Ioos et al., Med. Mal. Infect., 44:302 (2014).
Johnson et al., J. Virol., 73:783 (1999).
Kimura-Kuroda et al., J. Immunol., 141:3606 (1988).
Lanciotti et al., Emerg. Infect. Dis., 14:1232 (2008).
Larocca et al., Nature, ______:______ (2016).
Lazear et al., J. Virol., 90:4864 (2016).
Li et al., Neuron., 92:949 (2016).
Meier et al., PLoS Pathog., 5:e1000614 (2009).
Merino-Ramos et al., PLoS One, 9:e108056 (2014).
Metz et al., Methods Mol. Biol., 1426:297 (2016).
Miner et al., Cell. 165:1081 (2016).
Mlakar et al., N. Engl. J. Med., 374:951 (2016).
Musso, Emerg. Infect. Dis., 21:1887 (2015).
Ohtaki et al., Vaccine, 28:6588 (2010).
Oliveira Melo et al., Ultrasound Obstet. Gynecol., 47:6 (2016).
Partidos et al., Vaccine, 29:3067 (2011).
Pijlman, Biotechnol. J., 10:659 (2015).
Pinto Junior et al., Acta Med. Port., 28:760 (2015).
Prestwood et al., J. Virol., 86:12561 (2012).
Reed et al., Am. J. Epid., 27:493 (1938).
Sarathy et al., J. Gen. Virol., 96:3035 (2015).
Shawan et al., Nat. Sci., :37 (2015).
Spohn et al., Virol. J., 7:146 (2010).
Tesh et al., Emerg. Infect. Dis., 8:1392 (2002).
Thomas et al., Am. J. Trop. Med. Hyg., 81:825 (2009).
Ticconi et al., Pathog. Glob. Health, 110:262 (2016).
Wang et al. Vaccine, 30:2125 (2012).

What is claimed is:

1. A recombinant nucleic acid vector comprising a heterologous promoter operably linked to a nucleotide sequence encoding flavivirus prM/E, which vector lacks nucleic acid sequences encoding one or more of flavivirus NS1, NS2A, NS2B NS3, NS4A NS4B or NS5 and optionally lacks nucleic acid sequences encoding functional flavivirus capsid.

2. The recombinant vector of claim 1 wherein the heterologous promoter is a heterologous viral promoter. The recombinant vector of claim 1 which includes a portion of flavivirus capsid sequences.

4. The recombinant vector of claim 1 wherein the capsid sequence includes amino acids 98 to 112 of the capsid protein encoded by SEQ ID NO:1 or a protein having at least 80% amino acid sequence identity thereto.

5. The recombinant vector of claim 1 wherein the flavivirus is a Zika virus.

6. The recombinant vector of claim 1 wherein the prM/E sequences have at least 80% amino acid sequence identity to the prM/E sequences encoded by any one of SEQ ID Nos. 1-3 or 5.

7. The recombinant vector of claim 1 wherein the prWE sequences are operably linked to a heterologous secretion signal.

8. The recombinant vector of claim 7 wherein the heterologous secretion signal is a TPA, IL-2, IgG kappa light chain, CD33, or Oikosin secretion signal.

9. A vaccine comprising an effective amount of a flavivirus like particle comprising a lipid bilayer comprising flavivirus prM/E but which particle lacks one or more of flavivirus NS1, NS2A, NS2B, NS3, NS4A, NS4B or NS5 and optionally lacks functional flavivirus capsid.

10. The vaccine of claim 9 further comprising one or more adjuvants.

11. The vaccine of claim 10 wherein the adjuvant comprises alum, monophosphoryl lipid A (MPLA), squalene, aluminum hydroxide absorbed TLR4 agonist, dimethyldioctadecylammonium, tripalmitoyl-S-glyceryl cysteine, trehalose dibehenate, saponin, MF59, AS03, virosomes, AS04, CpG, imidazoquinoline, poly I:C, flagellin, or any combination thereof

12. The vaccine of claim 9 wherein the flavivirus is a Zika virus.

13. The vaccine of claim 9 wherein the prM/E sequences have at least 80% amino acid sequence identity to the prM/E sequences encoded by any one of SEQ ID Nos. 1-3 or 5.

14. A method to prevent, inhibit or treat flavivirus infection in a mammal, comprising: administering to the mammal a composition comprising an effective amount of a flavivirus like particle comprising a lipid bilayer comprising flavivirus prM/E but which particle lacks one or more of flavivirus NS1, NS2A, NS2B, NS3, NS4A, NS4B or NSS and optionally lacks functional flavivirus capsid, or a composition comprising an effective amount of anti-flavivirus antibodies.

13. The method of claim 14 wherein the mammal is a female mammal.

14. The method of claim 14 wherein the mammal is a human.

15. The method of claim 14 wherein the flavivirus is a Zika virus.

16. The method of claim 17 wherein the prM/E sequences have at least 80% amino acid sequence identity to the prM/E sequences encoded by any one of SEQ ID Nos. 1-3 or 5.

17. The method of claim 14 wherein the composition comprising the flavivirus like particle is administered intramuscularly, subcutaneously or intranasally.

18. The method of claim 14 wherein the composition inhibits flavivirus infection.

19. The method of claim 14 wherein the composition treats flavivirus infection.

20. The method of claim 14 wherein the composition comprising antibodies comprises antibodies pooled from multiple donors that were infected with the flavivirus.
